(19) United States
(12) Patent Application Publication
(10) Pub. No.: US 2025/0051282 A1
(43) Pub. Date: Feb. 13, 2025

(54) COMPOUNDS FOR TREATING POLYQ-RELATED NEURODEGENERATIVE DISORDERS

(71) Applicants: HANGZHOU JIJING PHARMACEUTICAL TECHNOLOGY LIMITED, Hangzhou, Zhejiang (CN); PAQ Therapeutics Inc., Burlington, MA (US)

(72) Inventors: Ning Yin, Lexington, MA (US); Nan Ji, Arlington, MA (US)

(21) Appl. No.: 18/717,301

(22) PCT Filed: Dec. 8, 2022
(86) PCT No.: PCT/CN2022/137686

§ 371 Date: Jun. 6, 2024

Related U.S. Application Data
(60) Provisional application No. 63/287,288, filed on Dec. 8, 2021.

(51) Int. Cl. C07D 209/34; A61K 31/4045; A61K 31/4155; A61K 31/4178; A61K 31/423; A61K 31/427; A61K 31/437; A61K 31/4439; A61K 31/454; A61K 31/4725; A61K 31/496; A61K 31/497; A61K 31/501; A61K 31/506; A61K 31/5377; C07D 401/06; C07D 401/12; C07D 403/06; C07D 403/12; C07D 405/06; C07D 405/12; C07D 413/06; C07D 413/14; C07D 417/06; C07D 417/14; C07D 471/04

(52) U.S. Cl. CPC C07D 209/34; A61K 31/4045; A61K 31/4155; A61K 31/4178; A61K 31/423; A61K 31/427; A61K 31/437; A61K 31/4439; A61K 31/454; A61K 31/4725; A61K 31/496; A61K 31/497; A61K 31/501; A61K 31/506; A61K 31/5377; C07D 401/06; C07D 401/12; C07D 403/06; C07D 403/12; C07D 405/06; C07D 405/12; C07D 413/06; C07D 413/14; C07D 417/06; C07D 417/14; C07D 471/04

(57) ABSTRACT

One embodiment of the disclosure is a compound represented by Formula (I) or a pharmaceutically acceptable salt thereof. The variables in Formula (I) are defined herein. Compounds of Formula (I) can be used to treat polyQ-related neurodegenerative disorders.

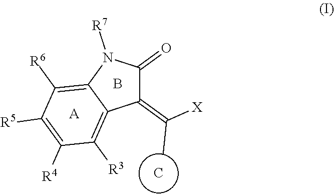

### RELATED APPLICATIONS

This application claims the benefit of the filing date, under 35 U.S.C. § 119(e), of U.S. Provisional Application No. 63/287,288, filed on Dec. 8, 2021, the entire contents of which is incorporated herein by reference.

### BACKGROUND

Neurodegenerative disorders are diseases caused by nervous system dysfunction resulting from abnormal death of central neurons. To date, there is a need for treatments that can slow down the development of such neurodegenerative disorders. Many neurodegenerative disorders are caused by proteins with unknown activity. Currently, methods that may be used to control protein levels, such as biological tools like RNAi, CRIPSR, etc. are difficult to deliver, especially to the nervous system.

A polyQ-related neurodegenerative disorder is a type of neurodegenerative disorder caused by mutant proteins, and may be treated by reducing the level of mutant proteins. Huntington's disease (HD), by example, is the most common neurodegenerative disorder. It is a monogenetic disorder wherein the mutation in the CAG repeat region of the exon1 of the HTT gene located in the patient's chromosome 4 results in the expansion of the polyglutamine (polyQ) of the synthesized mutant protein (mHTT). mHTT is susceptible to shearing and aggregating, and causes toxicity which eventually leads to dysfunction and death of specific neurons. The current methods for controlling mHTT levels through low molecular weight compounds lack specificity, and may cause side effects. Furthermore, these methods are nonallele-selective and unable to distinguish between mHTT and wild-type HTT protein (wtHTT), which leads to a decrease in the level of wtHTT which has important biological functions.

Similarly, spinocerebellar ataxia type 3 (SCA3; also known as Machado-Joseph disease, MJD) is the most common autosomal dominant spinocerebellar ataxia and is second only to HD as a common polyQ-related disorder in the world. SCA3 is caused by the abnormally expanded polyQ at the C-terminus of the coded protein ATXN3, resulting from the increase in the number of CAG repeats of the Ataxin-3 gene (ATXN3; also known as the MJD1 gene).

Some studies have used siRNA, antisense oligonucleotides, and other means to act on ATXN3 in order to reduce the expression of mutant ATXN3 protein, and it is confirmed that the reduction in the level of mutant ATXN3 protein can produce therapeutic effects (Wang, Neuroscience, 371, 2018, 138-154). Other studies have focused on controlling the level of mutant ATXN3 protein using low molecular weight compounds. Other studies have focused on enhancing autophagy using compounds (e.g., Menzies et al. Brain 2010, 133:93-104), while other studies have focused on reducing the level of mutant ATXN3 protein by regulating other targets (e.g., Costa MD, Brain, 2016, 139(11), 2891-2908). These studies, however, have not satisfactorily solved the problem of specificity.

A need exists to discover therapeutic compounds for treating polyQ-related neuro-degenerative disorders and diseases.

### SUMMARY

Provided herein are compounds, or pharmaceutically acceptable salts thereof, and compositions for treating polyQ-related neurodegenerative disorders and diseases. For example, the disclosed compounds promote degradation of Daoy-ATXN1(60Q)-mATXN1 with an expanded polyQ region in an MSD assay (see Table 1).

A first embodiment of the disclosure is a compound represented by Formula (I):

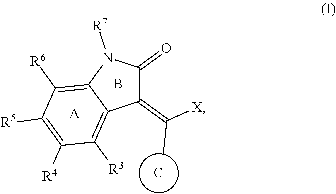

or a pharmaceutically acceptable salt thereof, wherein:
X is selected from NH₂, —N(R^{N})—R¹, ring D and —N(R^{N})-ring D and C₁₋₆ alkyl optionally substituted with one or more R¹⁰;
each R^{N} is independently selected from H, C₁₋₆ alkyl and C₃₋₇ cycloaliphatic;
R¹ is C₁₋₆ alkyl optionally substituted with one or more R¹⁰;
   each R¹⁰ is independently selected from halo, —CN, —OR¹⁵, —SR¹⁵, —S(O)R¹⁰⁰,
      —SO₂R¹⁰⁰, —S(O)N(R¹⁰⁰)₂, —SO₂N(R¹⁰⁰)₂, —OC(O)R¹⁰⁰, —OC(O)N(R¹⁰⁰)₂,
      —N(R¹⁰⁰)₂, —N(R¹⁰⁰)C(O)R¹⁰⁰, —N(R¹⁰⁰)CO₂R¹⁰⁰, —N(R¹⁰⁰)C(O)N(R¹⁰⁰)₂,
      —N(R¹⁰⁰)SO₂R¹⁰⁰, —C(O)R¹⁰⁰, —CO₂R¹⁰⁰, —C(O)N(R¹⁰⁰)₂, —C(O)N(R¹⁰⁰)OR¹⁵ and phenyl;
   each R¹⁵ is independently selected from H, C₁₋₆ alkyl and —(CH₂CH₂O)ₙ—R¹⁶
      wherein n is 1, 2, 3, 4 or 5;
   each R¹⁶ is independently selected from H, C₁₋₆ alkyl and —N(R^{N})C(O)R¹⁰⁰;
   each R¹⁰⁰ is independently selected from H, and C₁₋₆ alkyl;
ring C is selected from C₃₋₇ cycloaliphatic, C₆₋₁₀ aryl, 3 to 12-membered heterocyclyl, and 5 to 10-membered heteroaryl, each of which is optionally substituted with one or more R₂
   each R² is independently selected from halo, —CN, —NO₂, —OR²⁰⁰, —OC(O)N(R²⁰⁰)₂, —OC(O)R²⁰⁰, —SR²⁰⁰, —S(O)R²⁰⁰, —SO₂R²⁰⁰, —S(O)N(R²⁰⁰)₂, —SO₂N(R²⁰⁰)₂, —C(O)R²⁰⁰, —CO₂R²⁰⁰, —C(O)N(R²⁰⁰)₂, —C(O)N(R²⁰⁰)OR²⁰⁰, —N(R²⁰⁰)₂, —N(R²⁰⁰)C(O)R²⁰⁰, —N(R²⁰⁰)CO₂R²⁰⁰, —N(R²⁰⁰)C(O)N(R²⁰⁰)₂, —N(R²⁰⁰)SO₂R²⁰⁰, C₁₋₆ alkyl and C₁₋₆ haloalkyl;
   each R²⁰⁰ is independently selected from H and C₁₋₆ alkyl;
   alternatively two adjacent R², taken together with the carbon atoms of ring C to which they are attached, form a 3 to 7-membered heterocyclyl;
ring D is selected from C₃₋₇ cycloaliphatic, C₆₋₁₀ aryl, 3 to 12-membered heterocyclyl and 5 to 10-membered heteroaryl, each of which is optionally substituted by one or more R¹¹;
   each R¹¹ is independently selected from halo, —CN, —NO₂, —OR¹¹⁰, —SR¹¹⁰, —OC(O)R¹¹⁰, —OC(O)N(R¹¹⁰)₂, —S(O)R¹¹⁰, —SO₂R¹¹⁰, —S(O)N(R¹¹⁰)₂, —SO₂N(R¹¹⁰)₂, —N(R¹¹⁰)₂, —COR¹¹⁰, —CO₂R¹¹⁰, —N(R¹¹⁰)C(O)N(R¹¹⁰)₂, —C(O)N(R¹¹⁰)₂, —C(O)N(R¹¹⁰)OR¹¹⁰, C₁₋₆ alkyl and C₁₋₆ haloalkyl;
   each R¹¹⁰ is independently selected from H and C₁₋₆ alkyl;
   alternatively two adjacent R¹¹, taken together with the carbon atoms of ring D to which they are attached, form a 3 to 7-membered heterocyclyl;
alternatively one R¹⁰ and one R², taken together with their intervening atoms, form a 5- or 6-membered heterocyclyl ring;
R³ is selected from H, halo, C₁₋₆ alkyl, C₁₋₆ haloalkyl and C₃₋₇ cycloaliphatic;
R⁴ is selected from H, halo, —CN, —OR⁴⁰, C₁₋₆ alkyl and C₁₋₆ haloalkyl;
   R⁴⁰ is selected from H, C₁₋₆ alkyl and C₁₋₆ haloalkyl;
R⁵ is selected from H, halo, C₁₋₆ alkyl and C₁₋₆ haloalkyl;
R⁶ is selected from H, halo, C₁₋₆ alkyl and C₁₋₆ haloalkyl;
R⁷ is H or C₁₋₆ alkyl optionally substituted with —OR⁷⁰ or N(R^{N})₂; and
   R⁷⁰ is selected from H, halo and C₁₋₆ alkyl; and wherein:
(i) at least one of R³, R⁴, R⁵ and R⁶ is not H;
(ii) when X is NH₂, then ring C is C₃₋₇ cycloaliphatic, 3 to 12-membered heterocyclyl or 5 to 10-membered heteroaryl, each of which is optionally substituted with one or more R²;
(iii) when X is NH₂ or —N(R^{N})—R¹, then R³, R⁵ and R⁶ are all H; and, when X is NH₂, and ring C is phenyl, then the phenyl substituted with one or more R²; and
(iv) when X is —N(R^{N})-ring D, then ring C and ring D are not both optionally-substituted C₆₋₁₀ aryl; and, when X is —N(R^{N})-ring D and ring D is 9-10 membered bicyclic heteroaryl or an optionally N-substituted piperindyl, then R³, R⁵ and R⁶ are all H.

Another embodiment of the disclosure is a pharmaceutical composition comprising a pharmaceutically acceptable carrier or excipient and a compound represented by Formula (I) or a pharmaceutically acceptable salt thereof.

Another embodiment of the disclosure is a method for treating a subject with a polyQ-related neurodegenerative disorder, the method comprising administering to the subject an effective amount of a compound disclosed herein or a pharmaceutically acceptable salt thereof, or with an effective amount of a pharmaceutical composition disclosed herein. In one aspect, the compound used in the method is a compound represented by Formula (I), wherein the limitations (i)-(iv) of the first embodiment described above do not apply. In another aspect, the compound used in the method is a compound represented by Formula (I), wherein the limitations (i)-(iv) of the first embodiment described above do apply.

Another embodiment of the disclosure provides a use of a compound of any one of Formula (I), (II), (III), (IV), (V), (VI), (VII) and (VIII), or a pharmaceutically acceptable salt thereof, or the use of a pharmaceutical composition disclosed herein, in the preparation of a medicament for treating a subject with a polyQ-related neurodegenerative disorder. In one aspect, the compound used is a compound represented by Formula (I) wherein the limitations (i)-(iv) of the first embodiment described above do not apply. In another aspect, the compound used is a compound represented by Formula (I) wherein the limitations (i)-(iv) of the first embodiment described above do apply.

Another embodiment of the disclosed provides a compound of any one of Formula (I), (II), (III), (IV), (V), (VI), (VII) and (VIII), or a pharmaceutically acceptable salt thereof, or a pharmaceutical composition disclosed herein, which is used to treat a polyQ-related neurodegenerative disorder. In one aspect, the compound used is a compound represented by Formula (I) wherein the limitations (i)-(iv) of the first embodiment described above do not apply. In another aspect, the compound used is a compound represented by Formula (I) wherein the limitations (i)-(iv) of the first embodiment described above do apply.

### DETAILED DESCRIPTION

The disclosed compounds or pharmaceutically acceptable salts thereof are useful for treating polyQ-related neurodegenerative disorders and diseases. Such disorders and diseases include, for example, spinocerebellar ataxia type 1, spinocerebellar ataxia type 2, spinocerebellar ataxia type 3, spinocerebellar ataxia type 7, spinocerebellar ataxia type 12, spinocerebellar ataxia type 17, dentatorubral-pallidoluysian atrophy, Huntington's disease and spinal-bulbar muscular atrophy.

### I. COMPOUND EMBODIMENTS

The compounds or pharmaceutically acceptable salts thereof, as described herein, are useful for treating polyQ-related neurodegenerative disorders. Example embodiments include:

First embodiment: a compound represented by Formula (I):

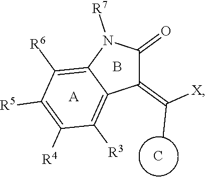

or a pharmaceutically acceptable salt thereof. The variables of Formula (I) are described in the Summary above.

Second embodiment: a compound represented by Formula (II):

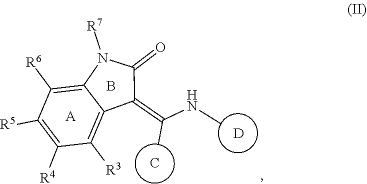

or a pharmaceutically acceptable salt thereof. The variables in Formula (II) are described in the first embodiment above or any specific embodiments therein.

Third embodiments: a compound represented by Formula (III):

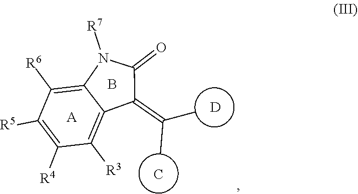

or a pharmaceutically acceptable salt thereof. The variables in Formula (III) are described in the first embodiment above or any specific embodiments therein.

Fourth embodiment: a compound of Formula (I), (II) or (III), or a pharmaceutically acceptable salt thereof, wherein ring D is selected from phenyl, C₃₋₆ cycloaliphatic, 4 to 6-membered heterocyclyl and 5 to 6-membered heteroaryl, each of which is optionally substituted by one or more R¹¹. The remaining variables in Formula (I), (II) or (III) are described in the first embodiment above or any specific embodiments therein.

Fifth embodiment: a compound of Formula (I), (II) or (III), or a pharmaceutically acceptable salt thereof, wherein ring D is a 5 to 6-membered heteroaryl optionally substituted by one or more R¹¹. The remaining variables in Formula (I), (II) or (III) are described in the first embodiment above or any specific embodiments therein.

Sixth embodiment: a compound of Formula (I), (II) or (III), or a pharmaceutically acceptable salt thereof, wherein ring D is selected from azetindinyl, cyclobutyl, cyclohexyl, cyclopentyl, cyclopropyl, imidazolyl, morpholinyl, phenyl, piperidinyl, piperizinyl, pyrazolyl, pyrazinyl, pyridinyl, pyridazinyl, pyrimidinyl, pyrrolidinyl and tetrahydrofuranyl, each of which is optionally substituted by one or more R¹¹. The remaining variables in Formula (I), (II) or (III) are described in the first embodiment above or any specific embodiments therein.

Seventh embodiment: a compound of Formula (I), (II) or (III), or a pharmaceutically acceptable salt thereof, wherein ring D is selected from pyrazolyl, pyridazinyl and pyrimidinyl, each of which is optionally substituted by one or more R¹¹. The remaining variables in Formula (I), (II) or (III) are described in the first embodiment above or any specific embodiments therein.

Eighth embodiment: a compound of Formula (I), (II) or (III), or a pharmaceutically acceptable salt thereof, wherein each R¹¹ is independently —OH, —O—C₁₋₄alkyl, —O—C₁₋₄haloalkyl, —OC(O)R¹¹⁰, —OC(O)N(R¹¹⁰)₂, —CO₂R¹¹⁰, —C(O)N(R¹¹⁰)₂, C₁₋₆ alkyl or C₁₋₆ haloalkyl, and each R¹¹⁰ is independently H or C₁₋₆ alkyl. The remaining variables in Formula (I), (II) or (III) are described in the first, second, third, fourth, fifth, sixth or seventh embodiment above or any specific embodiments therein.

Ninth embodiment: a compound of Formula (I), (II) or (III), or a pharmaceutically acceptable salt thereof, wherein each R¹¹ is independently —OH, —O—C₁₋₄alkyl, —CO₂R¹¹⁰ or C₁₋₆ alkyl, and each R¹¹⁰ is independently H or C₁₋₆ alkyl. The remaining variables in Formula (I), (II) or (III) are described in the first, second, third, fourth, fifth, sixth or seventh embodiment above or any specific embodiments therein.

Tenth embodiment: a compound of Formula (I), (II) or (III), or a pharmaceutically acceptable salt thereof, wherein each R¹¹ is independently —OH, —CH₃ or CO₂H. The remaining variables in Formula (I), (II) or (III) are described in the first, second, third, fourth, fifth, sixth or seventh embodiment above or any specific embodiments therein.

Eleventh embodiment: a compound represented by Formula (IV):

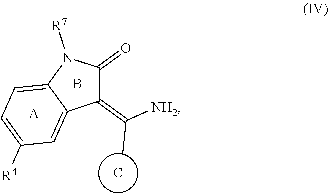

or a pharmaceutically acceptable salt. The variables in Formula (IV) are described in the first embodiment above or any specific embodiments therein.

Twelfth embodiment: a compound represented by Formula (V) or (VI):

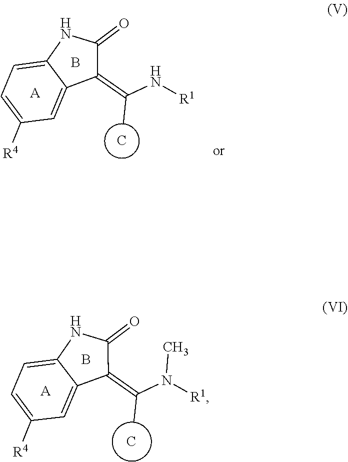

or a pharmaceutically acceptable salt thereof. The variables in Formula (V) or (VI) are described in the first embodiment above or any specific embodiments therein.

Thirteenth embodiment: a compound represented by Formula (VII):

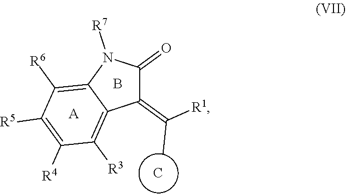

or a pharmaceutically acceptable salt thereof. The variables in Formula (VII) are described in the first embodiment above or any specific embodiments therein.

Fourteenth embodiment: a compound represented by Formula (VIII):

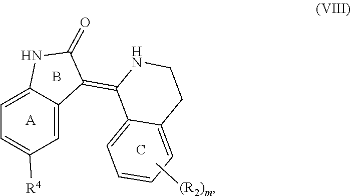

or a pharmaceutically acceptable salt thereof, wherein m is 0, 1, 2 or 3. The variables in Formula (VIII) are described in the first embodiment above or any specific embodiments therein.

Fifteenth embodiment: a compound of Formula (I), (V), (VI) or (VII), or a pharmaceutically acceptable salt thereof, wherein: R¹ is C₁₋₃ alkyl substituted with one or more R¹⁰; each R¹⁰ is independently selected from halo, —OR¹⁵, —N(R¹⁰⁰)₂ and phenyl; each R¹⁵ is independently selected from H, —CH₃ and —(CH₂CH₂O)ₙ—NHC(O)R¹⁰⁰; n is 1, 2, 3, 4 or 5; and each R¹⁰⁰ is independently H or —CH₃. The remaining variables in Formula (I), (V), (VI) or (VII) are described in the first embodiment above or any specific embodiments therein.

Sixteenth embodiment: a compound of Formula (I), (V), (VI) or (VII), or a pharmaceutically acceptable salt thereof, wherein each R¹⁰ is independently selected from F, —OH, —NH₂, —O—(CH₂CH₂O)ₙ—NHC(O)CH₃ and phenyl. The remaining variables in Formula (I), (V), (VI) or (VII) are described in the first embodiment above or any specific embodiments therein.

Seventeenth embodiment: a compound of Formula (I), (II), (III), (IV), (V), (VI) or (VII), or a pharmaceutically acceptable salt thereof, wherein ring C is selected from C₃₋₇ cycloaliphatic, C₆₋₁₀ aryl, 9-membered heterocyclyl and 5 to 9-membered heteroaryl, each of which is optionally substituted with one or more R². The remaining variables in Formula (I), (II), (III), (IV), (V), (VI) or (VII) are described in the first, fourth, fifth, sixth, seventh, eight, ninth, tenth, fifteenth or sixteenth embodiment above or any specific embodiments therein.

Eighteenth embodiment: a compound of Formula (I), (II), (III), (IV), (V), (VI) or (VII), or a pharmaceutically acceptable salt thereof, wherein ring C is the 5 to 9-membered heteroaryl optionally substituted with one or more R². The remaining variables in Formula (I), (II), (III), (IV), (V), (VI) or (VII) are described in the first, fourth, fifth, sixth, seventh, eight, ninth, tenth, fifteenth or sixteenth embodiment above or any specific embodiments therein.

Nineteenth embodiment: a compound of Formula (I), (II), (III), (IV), (V), (VI) or (VII), or a pharmaceutically acceptable salt thereof, wherein ring C is selected from benzofuranyl, 2,3-dihydrobenzo[b][1,4]dioxinyl, isoxazolyl, napthylenyl, phenyl, pyridinyl, pyrimidinyl, 1H-pyrrolo[2,3-b]pyridinyl and thiazolyl, each of which is optionally substituted with one or more R². The remaining variables in Formula (I), (II), (III), (IV), (V), (VI) or (VII) are described in the first, fourth, fifth, sixth, seventh, eight, ninth, tenth, fifteenth or sixteenth embodiment above or any specific embodiments therein.

Twentieth embodiment: a compound of Formula (I), (II), (III), (IV), (V), (VI) or (VII), or a pharmaceutically acceptable salt thereof, wherein ring C is phenyl optionally substituted with one or more R². The remaining variables in Formula (I), (II), (III), (IV), (V), (VI) or (VII) are described in the first, fourth, fifth, sixth, seventh, eight, ninth, tenth, fifteenth or sixteenth embodiment above or any specific embodiments therein.

Twenty-first embodiment: a compound of Formula (I), (II), (III), (IV), (V), (VI), (VII) or (VIII), or a pharmaceutically acceptable salt thereof, wherein each R² is independently selected from halo, —CN, —OR²⁰⁰, —OC(O)N(R²⁰⁰)₂, —OC(O)R²⁰⁰, —N(R²⁰⁰)₂, —N(R²⁰⁰)C(O)R²⁰⁰, —N(R²⁰⁰)CO₂R²⁰⁰, —N(R²⁰⁰)SO₂R²⁰⁰, C₁₋₄ alkyl and C₁₋₄ haloalkyl, and each R²⁰⁰ is independently selected from H and C₁₋₄ alkyl. The remaining variables in Formula (I), (II), (III), (IV), (V), (VI), (VII) or (VIII) are described in the first, fourth, fifth, sixth, seventh, eight, ninth, tenth, fifteenth, sixteenth, seventeenth, eighteenth, nineteenth or twentieth embodiment above or any specific embodiments therein.

Twenty-second embodiment: a compound of Formula (I), (II), (III), (IV), (V), (VI), (VII) or (VIII), or a pharmaceutically acceptable salt thereof, wherein each R² is independently selected from halo, —OR²⁰⁰, —N(R²⁰⁰)₂, —N(R²⁰⁰)C(O)R²⁰⁰, —N(R²⁰⁰)CO₂R²⁰⁰. —N(R²⁰⁰)SO₂R²⁰⁰, C₁₋₃ alkyl and C₁ haloalkyl, and each R²⁰⁰ is independently selected from H and C₁₋₃ alkyl. The remaining variables in Formula (I), (II), (III), (IV), (V), (VI), (VII) or (VIII) are described in the first, fourth, fifth, sixth, seventh, eight, ninth, tenth, fifteenth, sixteenth, seventeenth, eighteenth, nineteenth or twentieth embodiment above or any specific embodiments therein.

Twenty-third embodiment: a compound of Formula (I), (II), (III), (IV), (V), (VI), (VII) or (VIII), or a pharmaceutically acceptable salt thereof, wherein each R² is independently selected from F, Cl, Br, —OH, —OCH₃, —NH₂, —N(CH₃)₂, —NHC(O)H, —CH(CH₃)₂, —NHCO₂CH(CH₃)₂, —NHSO₂CH₃, —CH₃, —CHF₂ and —CF₃. The remaining variables in Formula (I), (II), (III), (IV), (V), (VI), (VII) or (VIII) are described in the first, fourth, fifth, sixth, seventh, eight, ninth, tenth, fifteenth, sixteenth, seventeenth, eighteenth, nineteenth or twentieth embodiment above or any specific embodiments therein.

Twenty-fourth embodiment: a compound of Formula (I), (II) or (III), or a pharmaceutically acceptable salt thereof, wherein R³ is selected from H, halo, C₁₋₄ alkyl and C₁₋₄ haloalkyl. The remaining variables in Formula (I), (II) or (III) are described in the first, fourth, fifth, sixth, seventh, eight, ninth, tenth, fifteenth, sixteenth, seventeenth, eighteenth, nineteenth, twentieth, twenty-first, twenty-second or twenty-third embodiment above or any specific embodiments therein.

Twenty-fifth embodiment: a compound of Formula (I), (II) or (III), or a pharmaceutically acceptable salt thereof, wherein R³ is H or —CH₃. The remaining variables in Formula (I), (II) or (III) are described in the first, fourth, fifth, sixth, seventh, eight, ninth, tenth, fifteenth, sixteenth, seventeenth, eighteenth, nineteenth, twentieth, twenty-first, twenty-second or twenty-third embodiment above or any specific embodiments therein.

Twenty-sixth embodiment: a compound of Formula (I), (II), (III), (IV), (V), (VI), (VII) or (VIII), or a pharmaceutically acceptable salt thereof, wherein R⁴ is selected from H, halo, —OR⁴⁰, C₁₋₃ alkyl and C₁₋₃ haloalkyl, where R⁴⁰ is C₁ haloalkyl. The remaining variables in Formula (I), (II), (III), (IV), (V), (VI), (VII) or (VIII) are described in the first, fourth, fifth, sixth, seventh, eight, ninth, tenth, fifteenth, sixteenth, seventeenth, eighteenth, nineteenth, twentieth, twenty-first, twenty-second, twenty-third, twenty-fourth or twenty-fifth embodiment above or any specific embodiments therein.

Twenty-seventh embodiment: a compound of Formula (I), (II), (III), (IV), (V), (VI), (VII) or (VIII), or a pharmaceutically acceptable salt thereof, wherein R⁴ is halo or a fluoromethoxy. The remaining variables in Formula (I), (II), (III), (IV), (V), (VI), (VII) or (VIII) are described in the first, fourth, fifth, sixth, seventh, eight, ninth, tenth, fifteenth, sixteenth, seventeenth, eighteenth, nineteenth, twentieth, twenty-first, twenty-second, twenty-third, twenty-fourth or twenty-fifth embodiment above or any specific embodiments therein.

Twenty-eighth embodiment: a compound of Formula (I), (II), (III), (IV), (V), (VI), (VII) or (VIII), or a pharmaceutically acceptable salt thereof, wherein R⁴ is selected from H, F, Cl, Br, —OCH₃, —OCHF₂, —OCF₃, —CH₃, —CH₂CH₃, —CF₃ and —CH(CH₃)₂. The remaining variables in Formula (I), (II), (III), (IV), (V), (VI), (VII) or (VIII) are described in the first, fourth, fifth, sixth, seventh, eight, ninth, tenth, fifteenth, sixteenth, seventeenth, eighteenth, nineteenth, twentieth, twenty-first, twenty-second, twenty-third, twenty-fourth or twenty-fifth embodiment above or any specific embodiments therein.

Twenty-ninth embodiment: a compound of Formula (I), (II), (III), (IV), (V), (VI), (VII) or (VIII), or a pharmaceutically acceptable salt thereof, wherein R⁴ is Br or —OCF₃. The remaining variables in Formula (I), (II), (III), (IV), (V), (VI), (VII) or (VIII) are described in the first, fourth, fifth, sixth, seventh, eight, ninth, tenth, fifteenth, sixteenth, seventeenth, eighteenth, nineteenth, twentieth, twenty-first, twenty-second, twenty-third, twenty-fourth or twenty-fifth embodiment above or any specific embodiments therein.

Thirtieth embodiment: a compound of Formula (I), (II) or (III), or a pharmaceutically acceptable salt thereof, wherein R⁵ is selected from H, halo and C₁₋₄ alkyl; R⁶ is selected from H, halo and C₁₋₄ alkyl; and R⁷ is H or C₁₋₄ alkyl optionally substituted with —OCH₃ or N(CH₃)₂. The remaining variables in Formula (I), (II) or (III) are described in the first, fourth, fifth, sixth, seventh, eight, ninth, tenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth or twenty-ninth embodiment above or any specific embodiments therein.

Thirty-first embodiment: a compound of Formula (I), (II) or (III), or a pharmaceutically acceptable salt thereof, wherein R⁵ is selected from H, Cl and —CH₃; R⁶ is selected from H, F and —CH₃; and R⁷ is selected from H, —CH₃, —CH₂CH₂OCH₃ and —CH₂CH₂N(CH₃)₂. The remaining variables in Formula (I), (II) or (III) are described in the first, fourth, fifth, sixth, seventh, eight, ninth, tenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth or twenty-ninth embodiment above or any specific embodiments therein.

Thirty-second embodiment: a compound of Formula (I), or a pharmaceutically acceptable salt thereof, wherein X is selected from NH₂, ring D and —N(R^{N})-ring D and C₁₋₆ alkyl optionally substituted with one or more R¹⁰. The remaining variables in Formula (I) are as described in the first embodiment above or any specific embodiments therein.

The disclosure also includes the compounds of Examples 1-84 that are described in the Exemplification, in their neutral form and pharmaceutically acceptable salts thereof.

### II. DEFINITIONS

As used herein, the term “alkyl” refers to a fully saturated branched or unbranched hydrocarbon moiety having 1 to 20 carbon atoms. Preferably, the alkyl comprises 1 to 6 carbon atoms, or 1 to 4 carbon atoms. Representative examples of alkyl include, but are not limited to, methyl, ethyl, 1-propyl, 2-propyl, 1-butyl, 2-methyl-1-propyl, 2-butyl, 2-methyl-2-propyl, 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, 1-heptyl, 1-octyl, and the like.

The terms “cycloaliphatic”, “cycloaliphatic group” or “cycloaliphatic ring” are used interchangeably to refer to a saturated (i.e., a cycloalkyl that is also defined below) or unsaturated non-aromatic, monocyclic or bicyclic carbon ring system (including fused and bridged bicyclic) which has 3- to 12-ring members, alternatively 3 to 7 members. The term “cycloaliphatic” also includes ring systems in which a carbocyclic (hydrocarbon) aromatic ring is fused to a saturated or partially unsaturated (non-aromatic) hydrocarbon ring. Examples of monocyclic cycloaliphatic ring systems include, but are not limited to, cyclopropyl, cyclobutyl, cyclopentyl, cyclohexyl, cycloheptyl, cyclobutenyl, cyclopenentyl, cyclohexenyl, cycloheptenyl and the like. Examples of bicyclic cycloaliphatic ring systems include, but are not limited to octahydronapthalenyl, decalinyl, and the like.

The term “cycloalkyl” refers to completely saturated monocyclic or bicyclic hydrocarbon rings (including fused and bridged bicyclic) having 3-12 ring carbon atoms, alternatively 3-7 ring carbon atoms. Exemplary bicyclic cycloalkyl groups include bicyclo[2.1.1]hexyl, bicyclo[2.2.1]heptyl, bicyclo[2.2.1]heptenyl, 6,6-dimethylbicyclo[3.1.1]heptyl, 2,6,6-trimethylbicyclo[3.1.1]heptyl and decalinyl. Examples of monocyclic cycloalkyl groups include, but are not limited to, cyclopropyl, cyclobutyl, cyclopentyl, cyclohexyl, cycloheptyl, cyclooctyl, cyclononyl, cyclodecyl, cycloundecyl, cyclododecyl, and the like.

The term “alkoxy” as used herein refers to the group —OR, in which R is an alkyl as defined above.

The terms “heterocyclyl,” “heterocycle,” and “heterocyclic ring” refer to a 3 to 12-membered (alternatively 3-7 membered), saturated or partially unsaturated (non-aromatic), monocyclic or bicyclic ring system (including fused and bridged bicyclic) having at least one or more heteroatom selected from O, S and N as a ring member, and wherein C can be oxidized (e.g., C(O)), N can be oxidized (e.g., N(O)) or quaternized, and S can be optionally oxidized to sulfoxide and sulfone. The term “heterocyclyl” includes ring systems in which an aryl or heteroaryl ring is fused to a saturated or partially unsaturated (non-aromatic) ring having at least one heteroatom as a ring member.

Examples of heterocyclyls include, but are not limited to, aziridinyl, oxiranyl, thirranyl, oxaziridinyl, azetidinyl, oxetanyl, tetrahydropyranyl, thietanyl, pyrrolidinyl, tetrahydrofuranyl, thiolanyl, imidazolidinyl, pyrazolidinyl, oxazolidinyl, 2-oxoimidazolidin-1-yl, isoxazolidinyl, thiazolidinyl, isothiazolidinyl, dioxolanyl, dithiolanyl, oxathiolanyl, piperidinyl, thianyl, piperazinyl, morpholinyl, thiomorpholinyl, dioxanyl, dithianyl, trioxanyl, trithianyl, azepanyl, oxepanyl, thiepanyl, dihydrofuranyl, imidazolinyl, dihydropyranyl, 2,3-dihydrobenzofuranyl, 2,3-dihydrobenzo[b][1,4]dioxinyl, indolinyl (or 2,3-dihydroindolyl), isoindolinyl, dihydro-pyrrolo pyridinyl, 1,3-dihydro-2H-pyrrolo[3,4-c]pyridin-2-yl, 5,7-dihydro-6H-pyrrolo[3,4-b]pyridin-6-yl, 2,3-dihydrobenzothiophenyl, 2,3-dihydrobenzothiazolyl, 1,2,3,4-tetrahydroquinolinyl, 1,2,3,4-tetrahydroisoquinolinyl, 5,6,7,8-tetrahydropyrido[3,4-b]pyrazinyl, 1,3-dihydroisobenzofuranyl, 6,7-dihydro-5H-pyrrolo[3,4-b]pyrazinyl, 1,3-dihydro-2H-pyrrolo[3,4-c]pyridin-2-yl, 5,7-dihydro-6H-pyrrolo[3,4-b]pyridin-6-yl, 6,7-dihydro-5H-pyrrolo[3,4-c]pyridazinyl, 6,7-dihydro-5H-pyrrolo[3,4-d]pyrimidinyl, and the like. Examples of bridged bicyclics include azabicyclo[2.2.1]hepantyl, azabicyclo[3.2.1]octanyl, azabicyclo[3.3.1]nonanyl, diazabicyclo[2.2.1]hepantyl, diazabicyclo[3.2.1]octanyl and diazabicyclo[3.3.1]nonanyl. Examples of oxygen containing bridged bicyclics include oxobicyclo[2.2.1]hepantyl, oxobicyclo[3.2.1]octanyl, oxobicyclo nonanyl, oxa-azabicyclo[2.2.1]hepantyl, oxa-azabicyclo[3.2.1]octanyl and oxa-azabicyclo [3.3.1]nonanyl.

The term “aryl” refers to an all-carbon monocyclic or fused polycyclic (such as bicyclic) aromatic ring group with a conjugated π-electron system. For example, aryl may have 6-14 carbon atoms, suitably 6-10, more suitably 6 or 10. Examples of aryl include, but are not limited to, phenyl, naphthyl, anthracenyl, etc.

As used herein, “heteroaryl” can be used interchangeably with “heteroaromatic,” “heteroaryl ring,” “heteroaryl group,” “heteroaromatic ring,” and “heteroaromatic group”. It refers to a 5 to 10-membered, fully aromatic, monocyclic or fused bicyclic ring system having at least one to four heteroatoms selected from O, N and S. Rings containing oxidized forms of N (e.g., N(O)) or S (e.g., sulfoxide and sulfone) are not encompassed within the meaning of “heteroaryl”. “Heteroaryl” includes monocyclic rings and bicyclic rings in which a monocyclic heteroaromatic ring is fused to a carbocyclic aromatic hydrocarbon or heteroaromatic ring.

Examples of heteroaryls include, but are not limited to, furanyl (e.g., 2-furanyl, 3-furanyl), imidazolyl (e.g., N-imidazolyl, 2-imidazolyl, 4-imidazolyl, 5-imidazolyl), isoxazolyl (e.g., 3-iso xazolyl, 4-isoxazolyl, 5-isoxazolyl), oxadiazolyl (e.g., 2-oxadiazolyl, 5-oxadiazolyl), oxazolyl (e.g., 2-oxazolyl, 4-oxazolyl, 5-oxazolyl), pyrazolyl (e.g., 3-pyrazolyl, 4-pyrazolyl), pyrrolyl (e.g., 1-pyrrolyl, 2-pyrrolyl, 3-pyrrolyl), pyridinyl (or pyridyl, e.g., 2-pyridyl, 3-pyridyl, 4-pyridyl), pyrimidinyl (e.g., 2-pyrimidinyl, 4-pyrimidinyl, 5-pyrimidinyl), pyridazinyl (e.g., 3-pyridazinyl), thiazolyl (e.g., 2-thiazolyl, 4-thiazolyl, 5-thiazolyl), isothiazolyl, triazolyl (e.g., 2-triazolyl, 5-triazolyl), tetrazolyl (e.g., tetrazolyl), thienyl (e.g., 2-thienyl, 3-thienyl), pyranyl, thiopyranyl, pyrazinyl, oxazinyl, thiazinyl, dioxinyl, dithiinyl, oxathianyl, triazinyl, tetrazinyl, azaindolyl, benzimidazolyl, benzofuryl, benzoisoxazolyl, benzoisothiazolyl, benzothiadiazolyl, benzothiazolyl, benzothienyl, benzotriazolyl, benzoxadiazolyl, benzoxazolyl, furopyridinyl, imidazopyridyl, imidazopyrimidinyl, indazolyl, indolizinyl, indolyl, isoquinolinyl, oxazolo pyridinyl, purinyl, pyrazolopyrimidinyl, pyrazolopyrazinyl, pyridopyazinyl, pyridopyrimidinyl, pyrrolo[2,3]pyrimidinyl, pyrrolopyrazolyl, pyrroloimidazolyl, pyrrolotriazolyl, quinazolinyl, quinoxalinyl, quinolinyl, isoquinolinyl, thiazolopyridinyl, thienopyridinyl, thienopyrimidinyl, thienopyrazinyl, napthyridyl, and the like.

The number of carbon atoms in a group is specified herein by the prefix “Cₓ₋ₓₓ”, wherein x and xx are integers. For example, “C₁₋₄alkyl” is an alkyl group which has from 1 to 4 carbon atoms.

The term “fused ring system”, as used herein, is a ring system that has two rings which share two adjacent ring atoms.

The term “bridged ring system”, as used herein, is a ring system wherein two non-adjacent atoms of the ring are connected (bridged) by one or more (preferably from one to three) atoms selected from C, N, O, and S. In one embodiment, a bridged ring system have from 6 to 12 ring members.

The term “Halogen” or “halo” as used herein refers to F, Cl, Br or I. Preferably, halo is F, Cl, or Br.

The term “haloalkyl” refers to an alkyl group having at least one halogen substitution. “Haloalkoxy” is a haloalkyl group which is attached to another moiety via an oxygen atom.

If a group is described as being “optionally substituted,” the group may be either (1) not substituted, or (2) substituted. If a carbon of a group is described as being optionally substituted with one or more of a list of substituents, one or more of the hydrogen atoms on the carbon (to the extent there are any) may separately and/or together be replaced with an independently selected optional substituent. If a group is substituted with one or more substituents, it can be substituted with 1, 2, 3, 4, 5, 6, or more independently selected substituents. In certain embodiments, it can be substituted with 1, 2, 3, 4, 5 or 6 independently selected substituents. In certain embodiments, it can be substituted with 1, 2 or 3 independently selected substituents.

The term “pharmaceutically acceptable” refers to that when contacted with the patient's tissue within the scope of normal medical judgment, no undue toxicity, irritation, allergic reactions, etc. shall arise, having reasonable advantage-disadvantage ratios and effective for the intended use.

The pharmaceutically acceptable salts of the compound of the present disclosure include acid addition salts and base addition salts thereof. Suitable acid addition salts are formed from acids that form pharmaceutically acceptable salts. Examples include hydrochloride, acetate, aspartate, benzoate, bicarbonate/carbonate, glucoheptonate, gluconate, nitrate, orotate, palmitic acid salt and other similar salt. Suitable base addition salts are formed from bases that form pharmaceutically acceptable salts. Examples include aluminum salts, arginine salts, choline salts, magnesium salts, and other similar salts. The method for preparing the pharmaceutically acceptable salt of the compound of the present disclosure is known to those skilled in the art.

The term “pharmaceutically acceptable carrier” refers to those substances that have no obvious stimulating effect on the organism and will not damage the biological activity and performance of the active compound. “Pharmaceutically acceptable carriers” include but are not limited to glidants, sweeteners, diluents, preservatives, dyes/colorants, flavors, surfactants, wetting agents, dispersants, disintegrants, stabilizer, solvent or emulsifier. Non-limiting examples of the carrier include calcium carbonate, calcium phosphate, various sugars and various starches, cellulose derivatives, gelatin, vegetable oils and polyethylene glycols, etc.

Compounds of the present disclosure may exist in various isomeric forms including geometric isomers of olefins, such as the olefin contained in compounds of Formula (I). Olefin isomers include the cis- and trans-olefin isomers (i.e., (Z) and (E) olefin isomers) and mixtures thereof. When the olefin configuration in compounds of Formula (I) is described as the (Z) or (E) olefin isomer by structure or by chemical name, the enrichment of the indicated olefin isomer relative to the other olefin isomer is greater than 50%, 60%, 70%, 80%, 90%, 99% or 99.9%.

“Enrichment of the indicated olefin isomer relative to the other olefin isomer” is a mole percent and is determined by dividing the number of compounds with the indicated olefin isomer by the total number of all of the compounds with the indicated or other olefin isomer in a mixture.

The term “administration” or “administrating” or the like refers to a method that enables a compound or composition to be delivered to a desired site of biological action. Such methods comprise but not limited to oral or parenteral (including intracerebroventricular, intravenous, subcutaneous, intraperitoneal, intramuscular, intravascular injection or infusion), local, rectal administration or the like. Especially injection or oral.

The terms “treatment,” “treat,” and “treating” refer to reversing, alleviating, or inhibiting the progress of a disclosed condition (e.g., neuro-degenerative disorder), or one or more symptoms thereof, as described herein. In some embodiments, treatment may be administered after one or more symptoms have developed, i.e., therapeutic treatment. In other embodiments, treatment may be administered in the absence of symptoms. For example, treatment may be administered to a susceptible individual prior to the onset of symptoms (e.g., in light of a history of symptoms and/or in light of genetic or other susceptibility factors), (i.e., prophylactic treatment) to reduce the likelihood of developing. Treatment may also be continued after symptoms have resolved, for example to prevent or delay their recurrence.

The term “effective amount” means an amount when administered to the subject which results in beneficial or desired results, including clinical results, e.g., inhibits, suppresses or reduces the symptoms of the condition being treated in the subject as compared to a control.

As used herein, the term “subject” includes a human or non-human animal. A subject includes a subject having a disease (such as one described herein) (which may be referred to as a “patient”), or a subject that is a susceptible subject prior to the onset of symptoms. Non-human animals include farm animals and companion animals.

### III. USE EMBODIMENTS

This disclosure provides methods for treating a subject with a polyQ-related neuro-degenerative disorder.

The term “neurodegenerative disorder” refers to a disease caused by the loss or pathological change of neurons and/or their myelin sheaths. Characteristic pathological structures, such as insoluble aggregates of protein, can be observed in the brain neurons of patients with neurodegenerative disorders. Insoluble aggregates may produce cytotoxicity, further leading to neuron loss and disease.

The term “polyQ” or “polyglutamine” refers to the polyglutamine tract contained by the protein. Glutamine is encoded by cytosine-adenine-guanine (CAG) in the gene. The length of the polyglutamine is related to the number of CAG repeats in gene exons. Therefore, the increase in the number of CAG repeats in gene exons result in polyglutamine expansions in the synthesized protein. Proteins with abnormally expanded polyQ are known to be associated with some neurodegenerative disorders. The gene name can use the form of “Q+number” to indicate the number of CAG repeats in exons, such as Q25 or Q72, which respectively indicate 25 repeats or 72 repeats of CAG in exons. In the protein name, the length of the polyglutamine can be expressed in the form of “Q+number” as above, such as Q27 or Q73, which means that the length of the polyglutamine is 27 Q (glutamine) or 73 Q, respectively. The CAG repetitions or glutamine repetitions indicated in the form of “Q+number” herein are all continuous repetitions. Unless otherwise specified, the length of polyQ as used herein refers to the length of the continuous polyglutamine.

The term “polyQ-related neurodegenerative disorder” refers to a neurodegenerative disorder associated with abnormal expansion of polyQ, or a neurodegenerative disorder that responds to levels of proteins containing expanded polyQ. Neurodegenerative disorders are a group of disorders with clinical and genetic heterogeneity. “Normal polyQ” refers to the polyQ of a protein in a normal physiological state that has a length less than a specific number. Correspondingly, “abnormally expanded” means that the polyQ of the protein has a length longer than the normal length. For diseases or pathological conditions, the length of polyQ is longer.

As an example, polyQ-related neurodegenerative disorders include but are not limited to pinocerebellar ataxia (SCA) type 1 (polyQ length≥41), type 2 (polyQ length≥34), type 3 polyQ length≥62), type 7 (polyQ length≥38), type 12 (polyQ length≥46), type 17 (polyQ length≥45); and dentatorubral-pallidoluysian atrophy (DRPLA, polyQ length≥49), untington's disease (HD, polyQ length≥36) and spinal-bulbar muscular atrophy (SBMA, polyQ length≥38). These diseases are caused by the expansion of CAG repeat regions on TXN1, ATXN2, ATXN3, ATXN7, ATXN12, TBP, ATN1, HTT and AR genes, respectively Lesley Jones et al., DNA repair in the trinucleotide repeat disorders, Lancet Neurol. 2017; 16: 88-96). Among them, spinocerebellar ataxia type 3 (SCA3, also known as Machado-Joseph disease, MJD) is the most common autosomal dominant spinocerebellar ataxia and is second only to HD as a common polyQ-related disorder in the world. SCA3 is caused by the abnormally expanded polyQ at the C-terminus of the coded protein ATXN3 resulting from the increase in the number of CAG repeats of the Ataxin-3 gene (ATXN3; also known as the MJD1 gene).

PolyQ-related neurodegenerative disorders treatable by the disclosed methods include, for example, spinocerebellar ataxia type 1, spinocerebellar ataxia type 2, spinocerebellar ataxia type 3, spinocerebellar ataxia type 7, spinocerebellar ataxia type 12, spinocerebellar ataxia type 17, dentatorubral-pallidoluysian atrophy, Huntington's disease and spinal-bulbar muscular atrophy.

Pharmaceutical compositions of the present disclosure may be in solid forms (e.g., powders, suspensions, tablets, capsules, wafers, patches, and the like), liquid forms (e.g., solutions, suspensions, elixirs, syrups, sprays, and the like) and semi-solid forms (e.g., lotions, gels, emulsions, and the like). Powders may include amorphous powders, crystalline powders, and mixtures thereof. Tablets may include chewable tablets, non-chewable tablets, ingestible tablets, buccal tablets, troches, lozenges, suppositories, and the like. Capsules may include hard-shell capsules and soft-shell capsules in the form of troches, lozenges, suppositories, and the like. Solutions include aqueous solutions, non-aqueous solutions and mixed (aqueous/non-aqueous) solutions in the form of parenteral solutions, injectable solutions, infusible solutions, and the like.

Dosage regimens can be adjusted to provide the desired optimal response. For example, when the medicine is administered in the form of injection, it can be administered as a single bolus injection, bolus injection and/or continuous infusion, etc. For example, several divided doses may be administered over time, or the dose may be proportionally reduced or increased as indicated by the urgent need of the treatment situation. It should be noted that the dose value may vary with the type and severity of the condition to be alleviated and may include single or multiple doses. Generally, the dose of treatment varies, depending on the considerations, such as the age, gender, and general health of the patient to be treated; the frequency of treatment and the nature of the desired effect; the degree of tissue damage; the duration of symptoms; and other variables that can be adjusted by individual physicians. It should be further understood that for any particular individual, the specific dosing regimen should be adjusted over time according to the needs of the individual and the professional judgment of the person administering the composition or supervising the administration of the composition. The dosage and regimen of the pharmaceutical composition can be easily determined by a person of ordinary skill in the clinical field. The required dose can be administered one or more times to obtain the desired result. The pharmaceutical composition according to the present disclosure can also be provided in unit dosage forms.

The composition can be formulated to contain a daily dose or an appropriate portion of the daily dose in a dosage unit, which can be a single tablet or capsule or a liquid of a suitable volume.

In one embodiment, the solution is prepared from a water-soluble salt, such as hydrochloride. Generally, all compositions are prepared according to known methods in pharmaceutical chemistry. Capsules can be prepared by mixing the compound with a suitable carrier or diluent and filling an appropriate amount of the mixture into the capsule. Commonly used carriers and diluents include, but are not limited to, inert powdered substances, such as a variety of different starches, powdered cellulose, especially crystalline and microcrystalline cellulose, sugars like fructose, mannitol and sucrose, cereal flour, and similar edible powder.

Tablets can be prepared by direct compression, wet granulation, or dry granulation. The preparation usually adds diluent, binder, lubricant and disintegrant and the compound. Typical diluents include, for example, various types of starch, lactose, mannitol, kaolin, calcium phosphate or sulfate, inorganic salts (such as sodium chloride) and powdered sugar. Powdered cellulose derivatives are also useful. Typical tablet binders are the following substances, such as starch, gelatin, and sugar (such as lactose, fructose, glucose, etc.). Natural and synthetic gums are also suitable, including gum acacia, alginate, methylcellulose, polyvinylpyrrolidone, etc. Polyethylene glycol, ethylcellulose and wax can also act as binders.

Lubricants can be selected from such slippery solids such as talc, magnesium stearate and calcium stearate, stearic acid and hydrogenated vegetable oils. A tablet disintegrant swells when wet to break the tablet and release the compound. They include starch, clay, cellulose, algin and gum. More specifically, for example, corn and potato starch, methylcellulose, agar, bentonite, lignocellulose, powdered natural sponge, anion exchange resin, alginic acid, guar gum, citrus pomace, carboxymethylcellulose and sodium lauryl sulfate can be used. Tablets can be coated with sugar as a flavoring and sealing agent or coated with a film-forming protective agent to optimize the dissolution performance of the tablet. The composition can also be formulated into chewable tablets, for example, by adding some substances to the formulation, such as mannitol.

When it is desired to be administered as a suppository, a typical base can be used. Cocoa butter is a traditional suppository base, which can be changed by adding wax to slightly increase its melting point. Especially water-miscible suppository bases including polyethylene glycols of various molecular weights are widely used.

The following detailed description of the disclosure is intended to illustrate non-limiting embodiments so that others skilled in the art can more fully understand the technical solution of the present disclosure, its principles and practical applications, and so that others skilled in the art can modify and implement the present disclosure in various manners that can be optimally adapted to the requirements of specific applications.

### EXEMPLIFICATION

The following examples are intended to be illustrative and are not meant in any way to be limiting.

### ABBREVIATIONS

Abbreviations and acronyms used herein include the following:
ACN acetonitrile
AcC1 acetyl chlorides
AcOH acetic acid (also “HOAc”)
Ac₂O acetic anhydride
Boc₂O di-tert-butyl dicarbonate
DAST diethylaminosulfur trifluoride
DCM dichloromethane (methylene chloride)
DIEA N,N-diisopropylethylamine
DMAc dimethylacetamide
DMF dimethylformamide
DMSO dimethylsulfoxide
Et₃N triethylamine
EtOAc ethyl acetate
HATU N-[(Dimethylamino)-1H-1,2,3-triazolo-[4,5-b]pyridin-1-ylmethylene]-N-methylmethanaminium hexafluorophosphate N-oxide
Lawesson's reagent 2,4-Bis(4-methoxyphenyl)-2,4-dithioxo-1,3,2,4-dithia diphosphetane
LCMS liquid chromatography-mass spectrometry
Pd(dppf)Cl₂ [1,1′-Bis(diphenylphosphino)ferrocene]dichloropalladium(II)
TBAF tetrbutylammonium fluoride
TBSCl tert-butyldimethylsilyl chloride
TBTU (2-(1H-benzotriazol-1-yl)-1,1,3,3-tetramethyluronium hexafluoro phosphate
TFA trifluoroaceticacid
THF tetrahydrofuran
TLC thin layer chromatography
TsOH p-toluenesulfonic acid

### General Synthetic Procedure

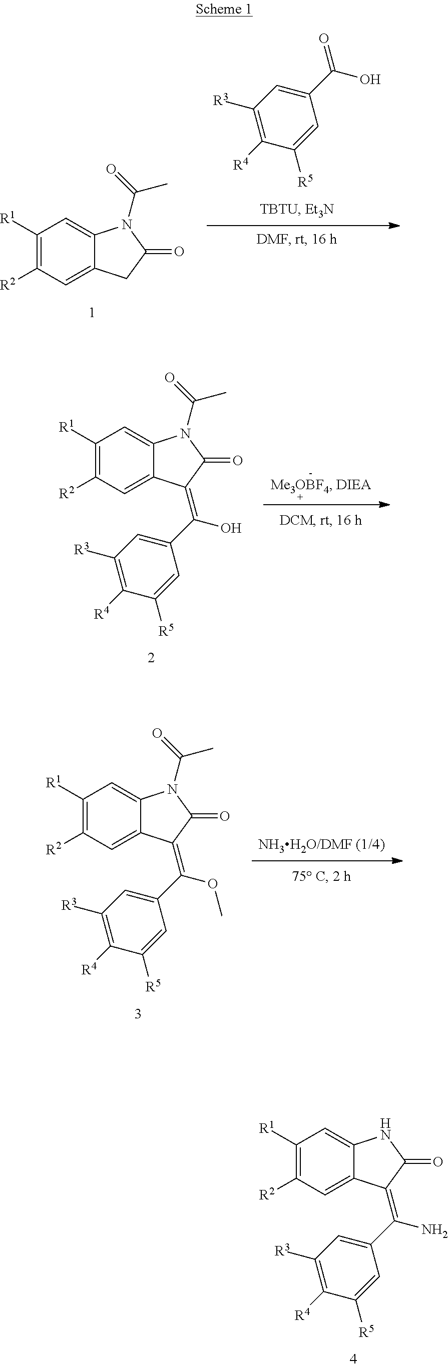

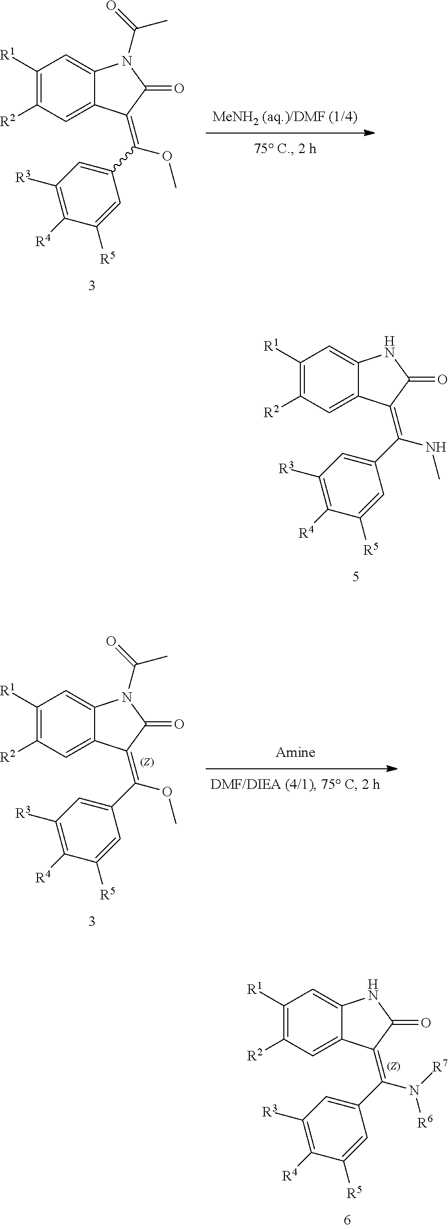

### General Procedure for Compound 2

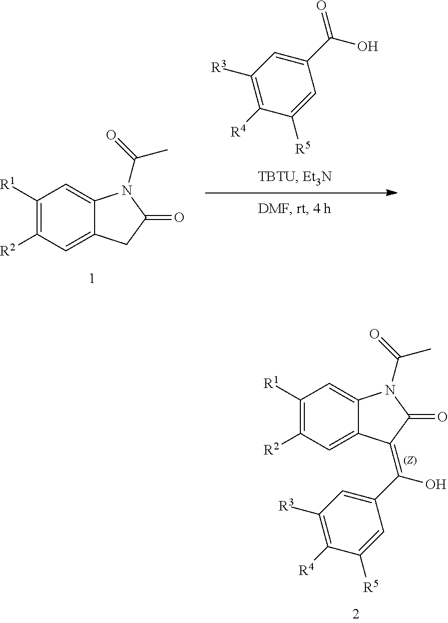

To a solution of Indolinone 1 (1 mmol) and Acid in DMF (5 mL) were added TBTU (1.3 mmol) and Et₃N (2.3 mmol) dropwise at room temperature. After stirring for 4 h at room temperature under nitrogen atmosphere, the resulting mixture was diluted with water and extracted with EtOAc. The combined organic layers were washed with brine, dried over anhydrous Na₂SO₄. After filtration, the filtrate was concentrated under reduced pressure. The residue was purified by reverse phase flash chromatography or silica gel column chromatography. Desired fractions were collected and concentrated under reduced pressure to afford Compound 2.

### General Procedure for Compound 3

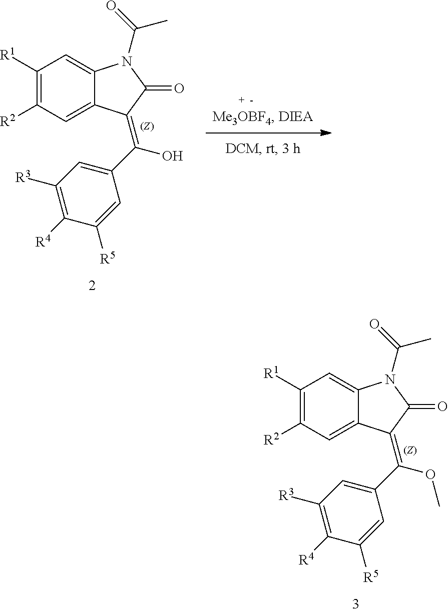

To a solution of Compound 2 (1 mmol) and DIEA (4 mmol) in DCM (12 mL) was added trimethyloxonium tetrafluoroborate (6 mmol) in portions at 0° C. under nitrogen atmosphere. After stirring for 3 h at room temperature under nitrogen atmosphere, the resulting mixture was diluted with DCM. The resulting mixture was washed with brine. The organic layer was dried over anhydrous Na₂SO₄. After filtration, the filtrate was concentrated under reduced pressure. The residue was purified by silica gel column chromatography, desired fractions were collected and concentrated under reduced pressure to afford Compound 3.

### General Procedure for Compound 4

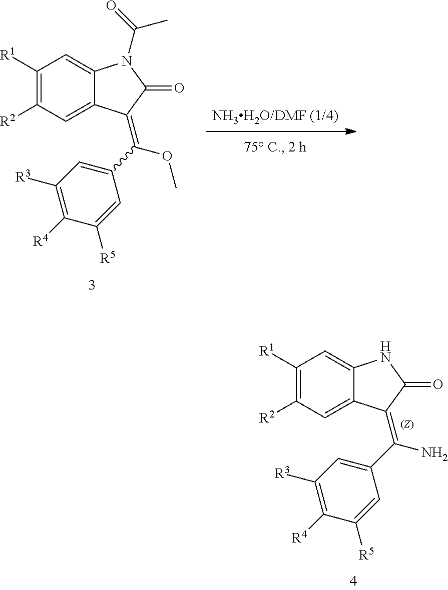

A solution of Compound 3 in DMF/NH₃·H₂O (v/v, 4/1) was stirred for 2 h at 75° C. under nitrogen atmosphere. The resulting mixture was purified by reversed phase flash chromatography. The fractions containing desired product were collected and concentrated under reduced pressure to afford Compound 4.

### General Procedure for Compound 5

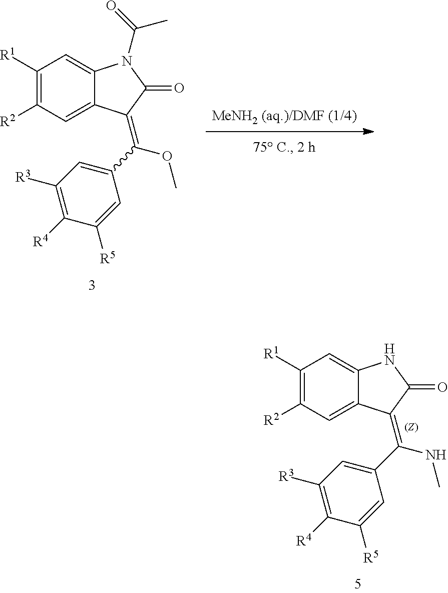

A solution of Compound 3 in DMF/MeNH₂ (aq.) (v/v, 4/1) was stirred for 2 h at 75° C. under nitrogen atmosphere. The resulting mixture was purified by reversed phase flash chromatography. The fractions containing desired product were collected and concentrated under reduced pressure to afford Compound 5.

### General Procedure for Compound 6

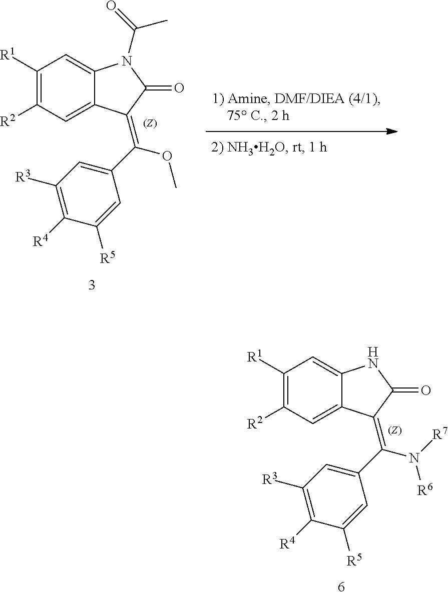

A solution of Compound 3 (0.20 mmol) and Amine (0.60 mmol) in DMF/DIEA (v/v, 4/1, 2 mL) was stirred for 2 h at 75° C. under nitrogen atmosphere. After cooling down to ambient temperature, NH₃·H₂O (0.2 mL) was added to the reaction mixture. The resulting mixture was stirred for additional 1 h at ambient temperature. The resulting mixture was purified by reversed phase flash chromatography. The fractions containing desired product were collected and concentrated under reduced pressure to afford Compound 6.

### Preparation of Acid Intermediates

### Preparation of 3-(Methoxymethoxy)-4-nitrobenzoic acid

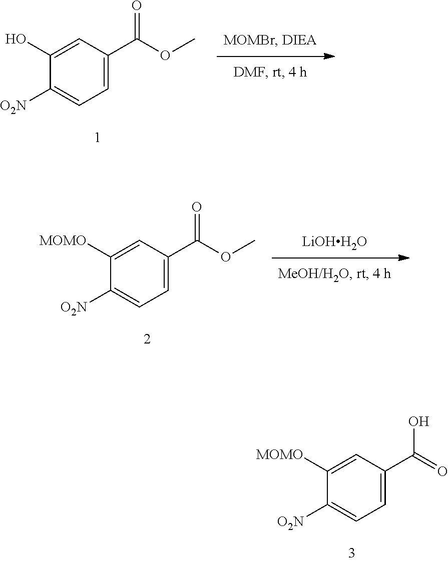

### Preparation of Methyl 3-(methoxymethoxy)-4-nitrobenzoate

To a stirred mixture of methyl 3-hydroxy-4-nitrobenzoate (10 g, 50.72 mmol) and DIEA (32.78 g, 253.62 mmol) in DMF (200 mL) was added bromo(methoxy)methane (12.68 g, 101.45 mmol) dropwise at 0° C. under nitrogen atmosphere. The resulting mixture was stirred for 4 h at room temperature under nitrogen atmosphere. The resulting mixture was diluted with water (1 L). The resulting mixture was extracted with EtOAc (3×400 mL). The combined organic layers were washed with brine (2×400 mL), dried over anhydrous Na₂SO₄. After filtration, the filtrate was concentrated under reduced pressure. The residue was purified by silica gel column chromatography, eluted with petroleum ether/EtOAc (5:1) to afford methyl 3-(methoxymethoxy)-4-nitrobenzoate (11.89 g, 98%) as a yellow solid: ¹H NMR (400 MHz, DMSO-d₆) δ 8.01 (d, J=8.4 Hz, 1H), 7.91 (d, J=1.7 Hz, 1H), 7.73 (dd, J=8.4, 1.7 Hz, 1H), 5.45 (s, 2H), 3.91 (s, 3H), 3.44 (s, 3H); LC/MS (ESI, m/z): [(M−1)]⁻=240.25.

### Preparation of 3-(Methoxymethoxy)-4-nitrobenzoic acid

A mixture of methyl 3-(methoxymethoxy)-4-nitrobenzoate (11.6 g, 48.09 mmol) and LiOH·H₂O (3.46 g, 144.27 mmol) in MeOH (230 mL) and H₂O (23 mL) was stirred for 4 h at room temperature under nitrogen atmosphere. The mixture was acidified to pH 6 with 3 M HCl (aq.). The resulting mixture was concentrated under reduced pressure. The residue was purified by reverse flash chromatography with the following conditions: Column: Spherical C18, 20-40 μm, 330 g; Eluent A: Water (plus 0.1% HCOOH), Eluent B: ACN; Flow rate: 80 mL/min; Gradient (B %): 5%-5%, 4 min; 5%-25%, 3 min; 25%-55%, 4 min; 55%-65%, 12 min; 65%-95%, 1 min; Detector: UV 254 nm. The fractions containing desired product were collected at 62% B and concentrated under reduced pressure to afford 3-(methoxymethoxy)-4-nitrobenzoic acid (9.5 g, 87%) as a yellow solid: ¹H NMR (400 MHz, DMSO-d₆) δ 7.87 (d, J=1.4 Hz, 1H), 7.80 (d, J=8.3 Hz, 1H), 7.65 (dd, J=8.2, 1.5 Hz, 1H), 5.35 (s, 2H), 3.42 (s, 3H); LC/MS (ESI, m/z): [(M+1)]⁺=228.25.

### Preparation of 12-(2,5-dimethyl-1H-pyrrol-1-yl)thiazole-4-carboxylic acid

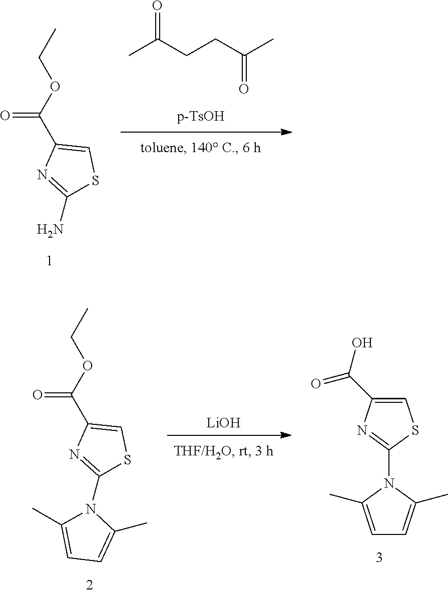

### Preparation of Ethyl 2-(2,5-dimethyl-1H-pyrrol-1-yl)thiazole-4-carboxylate

To a stirred solution of ethyl 2-amino-1,3-thiazole-4-carboxylate (5 g, 29.04 mmol) and 2,5-hexanedione (6.63 g, 58.07 mmol) in toluene (100 mL) was added TsOH·H₂O (0.55 g, 2.90 mmol) at room temperature under nitrogen atmosphere. The resulting mixture was refluxed for 6 h at 140° C. under nitrogen atmosphere with a Dean-Stark trap to collect the water generated in the reaction. The mixture was allowed to cool down to room temperature. The resulting mixture was concentrated under reduced pressure. The residue was purified by silica gel column chromatography, eluted with petroleum ether in EtOAc (0-30%) to afford ethyl 2-(2,5-dimethyl-1H-pyrrol-1-yl)thiazole-4-carboxylate (6.9 g, 95%) as a yellow oil: ¹H NMR (400 MHz, CDCl₃) δ 8.18 (s, 1H), 5.88 (s, 2H), 4.44 (q, J=7.1 Hz, 2H), 2.22 (s, 6H), 1.42 (t, J=7.1 Hz, 3H); LC/MS (ESI, m/z): [(M+1)]⁺=251.15.

### Preparation of 2-(2,5-Dimethyl-1H-pyrrol-1-yl)thiazole-4-carboxylic acid

A solution of ethyl 2-(2,5-dimethylpyrrol-1-yl)-1,3-thiazole-4-carboxylate (600 mg, 2.40 mmol) and LiGH (230 mg, 9.59 mmol) in THF (8 mL) and H₂O (2 mL) was stirred for 3 h at room temperature under nitrogen atmosphere. The mixture was acidified to pH 6 with acetic acid. The resulting mixture was concentrated under reduced pressure. The residue was purified by silica gel column chromatography, eluted with 0-40% MeOH in DCM to afford 2-(2,5-dimethyl-1H-pyrrol-1-yl)thiazole-4-carboxylic acid (500 mg, 94%) as a yellow oil: ¹H NMR (400 MHz, CDCl₃) δ 8.16 (s, 1H), 5.89 (s, 2H), 2.20 (s, 6H); LC/MS (ESI, m/z): [(M+1)]⁺=223.05.

### Preparation of 3-(Difluoromethyl)benzoic acid

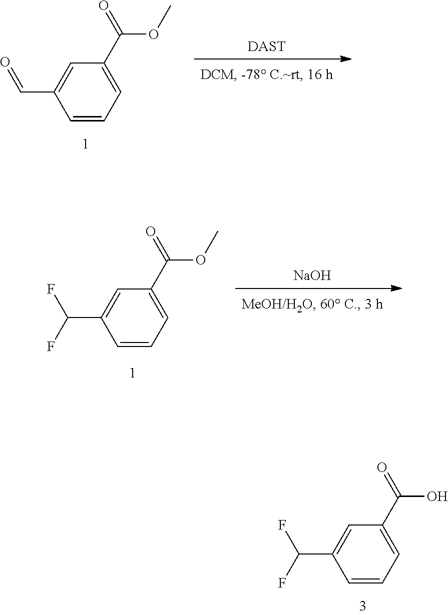

### Preparation of Methyl 3-(difluoromethyl)benzoate

To a stirred mixture of methyl 3-formylbenzoate (4.00 g, 24.37 mmol) in DCM (50 mL) was added DAST (11.78 g, 73.10 mmol) dropwise at −78° C. under nitrogen atmosphere. The resulting mixture was stirred for 16 h at room temperature under nitrogen atmosphere. The reaction was quenched by the addition of sat. NaHCO₃ (aq.) (15 mL) at −78° C. The resulting mixture was diluted with water (100 mL) and extracted with EtOAc (3×100 mL). The combined organic layers were washed with brine (2×50 mL), dried over anhydrous Na₂SO₄. After filtration, the filtrate was concentrated under reduced pressure to give crude product methyl 3-(difluoromethyl)benzoate (3.20 g, 71%) as an off-white solid which was used in the next step directly without further purification: LC/MS (ESI, m/z): [(M+1)]⁺=187.10.

### Preparation of 3-(Difluoromethyl)benzoic acid

To a stirred mixture of methyl 3-(difluoromethyl)benzoate (3.00 g, 16.12 mmol) in MeOH (30 mL) was added NaOH (900 mg, 24.17 mmol) in portions at room temperature under nitrogen atmosphere. The resulting mixture was stirred for 3 h at 60° C. under nitrogen atmosphere. After cooling down to ambient temperature, the mixture was acidified to pH 5 with 3 M HCl (aq.). The resulting mixture was concentrated under reduced pressure. The residue was purified by reverse flash chromatography with the following conditions: Column: Spherical C18, 20-40 μm, 330 g; Eluent A: Water (plus 0.1% HCOOH), Eluent B: ACN; Flow rate: 80 mL/min; Gradient (B %): 5%-30%, 15 min; 30%-40%, 15 min; 40%-95%; 2 min; 95%, 5 min; Detector: UV 254 nm. The fractions containing desired product were collected at 30% B and concentrated under reduced pressure to afford 3-(difluoromethyl)benzoic acid (2.5 g, 91%) as an off-white solid: 1H NMR (400 MHz, DM-d₆) δ8.17-8.07 (m, 2H), 7.81 (d, J=7.8 Hz, 1H), 7.65 (t, J=7.7 Hz, 1H), 7.13 (t, J=55.7 Hz, 1H); LC/MS (ESI, m/z): [(M−1)]⁻=170.90.

The following intermediates were prepared using the Preparation of Acid Intermediates described above.

| | | MS: | |
|---|---|---|---|
| Structure | Name | [(M − 1)]⁻ | ¹H NMR |
| 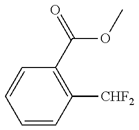 | Methyl 2- (difluoromethyl) benzoate | — | ¹H NMR (400 MHz, CDCl₃) δ 8.06 (dd, J = 7.8, 1.3 Hz, 1H), 7.89-7.82 (m, 1H), 7.72-7.64 (m, 1H), 7.62-7.51 (m, 1H), 7.56 (t, J = 55.6 Hz, 1H ), 3.96 (s, 3H). |
| 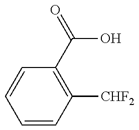 [SMILES: O=C(O)c1ccccc1C(F)F] | 2-(Difluoromethyl) benzoic acid | 171.10 | ¹H NMR (400 MHz, CDCl₃) δ 8.19 (dd, J = 7.9, 1.5 Hz, 1H), 7.88 (d, J = 7.8 Hz, 1H), 7.76- 7.69 (m, 1H), 7.65-7.56 (m, 1H), 7.59 (t, J = 55.4 Hz, 1H). |
| 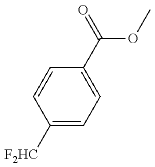 | Methyl 4- (difluoromethyl) benzoate | — | ¹H NMR (400 MHz, CDCl₃) δ 8.12 (d, J = 8.0 Hz, 2H), 7.59 (d, J = 8.0 Hz, 2H), 6.69 (t, J = 56.1 Hz, 1H), 3.94 (s, 3H). |
| 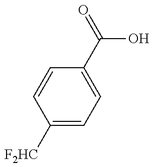 [SMILES: O=C(O)c1ccc(C(F)F)cc1] | 4- (Difluoromethyl) benzoic acid | 171.10 | ¹H NMR (400 MHz, DMSO- d₆) δ 13.23 (s, 1H), 8.07 (d, J = 8.0 Hz, 2H), 7.70 (d, J = 8.0 Hz, 2H), 7.13 (t, J = 55.6 Hz, 1H). |

### Preparation of Key Intermediates A and B

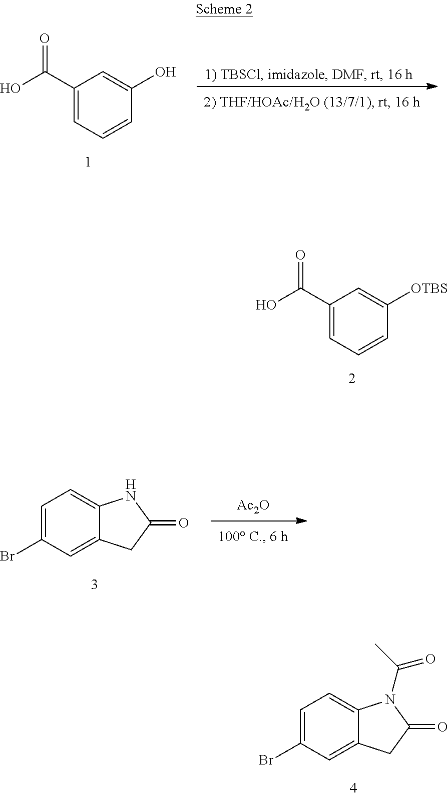

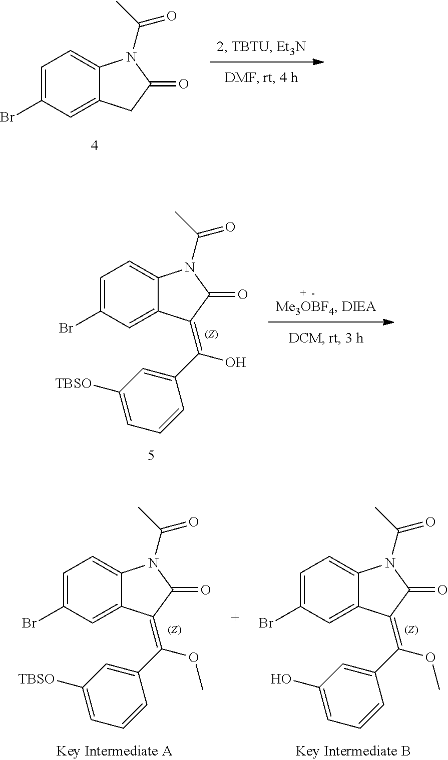 [SMILES: CC(=O)N1C(=O)Cc2cc(Br)ccc21 ; "2, TBTU, Et3N" ; CC(=O)N1C(=O)/C(=C(\O)c2cccc(O[Si](C)(C)C(C)(C)C)c2)c2cc(Br)ccc21 ; "Me3OBF4, DIEA" ; CO/C(=C1\C(=O)N(C(C)=O)c2ccc(Br)cc21)c1cccc(O[Si](C)(C)C(C)(C)C)c1 ; CO/C(=C1\C(=O)N(C(C)=O)c2ccc(Br)cc21)c1cccc(O)c1]

### Preparation of 3-((Tert-butyldimethylsilyl)oxy)benzoic acid (2)

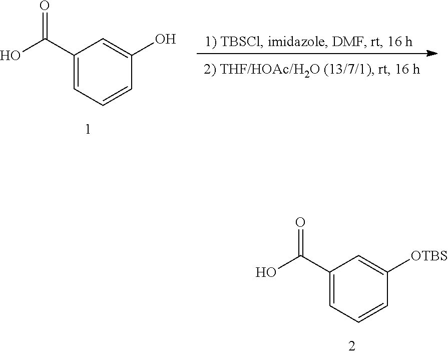

To a stirred mixture of 3-hydroxybenzoic acid (20 g, 144.80 mmol) in DMF (400 mL) were added Imidazole (34.4 g, 505.31 mmol) and TBSCl (65.2 g, 432.58 mmol) at 0° C. under nitrogen atmosphere. The resulting mixture was stirred for overnight at room temperature under nitrogen atmosphere. The resulting mixture was diluted with water (1.2 L). The resulting mixture was extracted with EtOAc (3×500 mL). The combined organic layers were washed with brine (2×500 mL), dried over anhydrous Na₂SO₄. After filtration, the filtrate was concentrated under reduced pressure. The residue was resolved in THF/HOAc/H₂O (13/7/1) (1 L). After stirring for 16 h at room temperature under nitrogen atmosphere. The resulting mixture was concentrated under reduced pressure. The residue was purified by silica gel column chromatography, eluted with petroleum ether/EtOAc (2:1) to afford 3-((tert-butyldimethylsilyl)oxy)benzoic acid (33.2 g, 91%) as a white solid: ¹H NMR (400 MHz, DMSO-d₆) δ 12.93 (s, 1H), 7.55 (dt, J=7.7, 1.3 Hz, 1H), 7.38 (t, J=7.8 Hz, 1H), 7.13-7.07 (m, 1H), 0.95 (s, 9H), 0.20 (s, 6H); LC/MS (ESI, m/z): [(M−1)]⁻=251.05.

### Preparation of 1-Acetyl-5-bromoindolin-2-one (4)

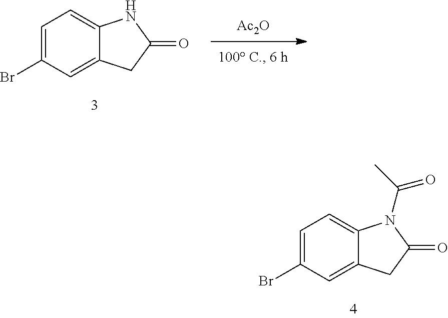

A solution of 5-bromo-1,3-dihydroindol-2-one (10 g, 47.16 mmol) in acetic anhydride (200 mL) was stirred for 6 h at 100° C. under nitrogen atmosphere. The resulting mixture was concentrated under reduced pressure. The residue was purified by trituration with petroleum ether/DCM/MeOH (40/2/0.5) to afford 1-acetyl-5-bromoindolin-2-one (7.5 g, 63%) as a light brown solid: ¹H NMR (400 MHz, DMSO-d₆) δ 8.00 (d, J=8.7 Hz, 1H), 7.56-7.54 (m, 1H), 7.52-7.48 (m, 1H), 3.84 (s, 2H), 2.55 (s, 3H); LC/MS (ESI, m/z): [(M−1)]⁻=251.80.

### Preparation of (Z)-1-acetyl-5-bromo-3-((3-((tert-butyldimethylsilyl)oxy)phenyl) (hydroxy)methylene)indolin-2-one (5)

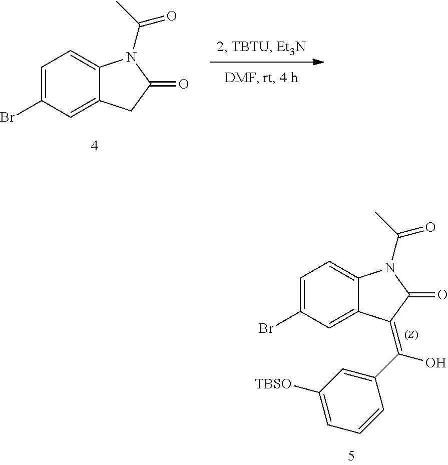 [SMILES: CC(=O)N1C(=O)Cc2cc(Br)ccc21 ; "2, TBTU, Et3N" ; CC(=O)N1C(=O)/C(=C(\O)c2cccc(O[Si](C)(C)C(C)(C)C)c2)c2cc(Br)ccc21]

To a stirred mixture of 1-acetyl-5-bromoindolin-2-one (6.00 g, 23.61 mmol) and 3-((tert-butyldimethylsilyl)oxy)benzoic acid (6.00 g, 23.77 mmol) in DMF (125 mL) were added TBTU (8.27 g, 25.75 mmol) and Et₃N (4.61 g, 45.57 mmol) dropwise room temperature under nitrogen atmosphere. The resulting mixture was stirred for 4 h at room temperature under nitrogen atmosphere. The resulting mixture was diluted with water (1 L). The resulting mixture was extracted with EtOAc (3×300 mL). The combined organic layers were washed with brine (2×300 mL), dried over anhydrous Na₂SO₄. After filtration, the filtrate was concentrated under reduced pressure. The residue was purified by reverse phase flash chromatography with the following conditions: Column: WelFlash TM C18-I, 20-40 m, 330 g; Eluent A: Water (plus 0.1% HCOOH); Eluent B: ACN; Gradient: 5%-5% B in 4 min; 5%-25% B in 4 min; 25%-55% B in 3 min; 55%-75% B in 3 min; 75%-85% B in 3 min; 85%-95% B in 8 min; Flow rate: 85 mL/min; Detector: UV 220/254 nm. The fractions containing desired product were collected at 95% B and concentrated under reduced pressure to afford (Z)-1-acetyl-5-bromo-3-((3-((tert-butyldimethylsilyl)oxy)phenyl)(hydroxy)methylene)indolin-2-one (6.30 g, 55%) as a light brown semi-solid: ¹H NMR (400 MHz, DMSO-d₆) δ 8.03 (s, 1H), 7.96 (d, J=8.5 Hz, 1H), 7.26 (t, J=7.8 Hz, 1H), 7.11 (d, J=7.6 Hz, 1H), 7.06 (d, J=8.4 Hz, 1H), 6.98 (t, J=2.0 Hz, 1H), 6.90-6.84 (m, 1H), 2.48 (s, 3H), 0.95 (s, 9H), 0.20 (s, 6H); LC/MS (ESI, m/z): [(M+1)]⁺=488.30.

The following intermediates were prepared using the synthetic procedures of the compounds 1-5 described above from the corresponding acid and indolinone.

| | | MS: | |
|---|---|---|---|
| Structure | Name | [(M + 1)]⁺ | ¹H NMR |
| 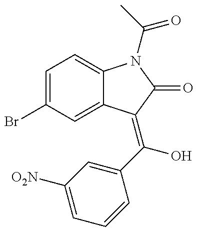 [SMILES: CC(=O)N1C(=O)/C(=C(\O)c2cccc([N+](=O)[O-])c2)c2cc(Br)ccc21] | (Z)-1-acetyl-5-bromo-3- (hydroxy(3-nitrophenyl) methylene)indolin-2-one | 402.90 | ¹H NMR (400 MHz, DMSO- d₆) δ 8.40-8.30 (m, 2H), 8.11 (d, J = 2.2 Hz, 1H), 8.08-7.97 (m, 2H), 7.75 (t, J = 7.9 Hz, 1H), 7.28 (dd, J = 8.6, 2.2 Hz, 1H), 2.46 (s, 3H). |
| 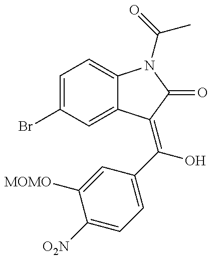 | (Z)-1-acetyl-5-bromo-3- (hydroxy(3-(methoxy methoxy)-4-nitrophenyl) methylene)indolin-2-one | 462.90 | ¹H NMR (400 MHz, DMSO- d₆) δ 8.08-8.01 (m, 2H), 7.95 (d, J = 8.3 Hz, 1H), 7.58 (d, J = 1.6 Hz, 1H), 7.36-7.29 (m, 2H), 5.37 (s, 2H), 3.43 (s, 3H), 2.48 (s, 3H). |
| 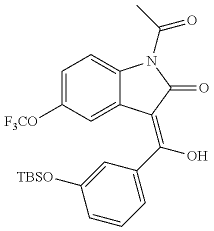 [SMILES: CC(=O)N1C(=O)/C(=C(\O)c2cccc(O[Si](C)(C)C(C)(C)C)c2)c2cc(OC(F)(F)F)ccc21] | (Z)-1-acetyl-3-((3-((tert- butyldimethylsilyl)oxy) phenyl)(hydroxy)methylene)- 5-(trifluoromethoxy) indolin-2-one | 494.10 | ¹H NMR (400 MHz, DMSO- d₆) δ 8.20 (d, J = 8.8 Hz, 1H), 7.64 (s, 1H), 7.45-7.37 (m, 1H), 7.21-7.11 (m, 2H), 7.07- 7.00 (m, 2H), 2.51 (s, 3H), 0.97 (s, 9H), 0.22 (s, 6H). |
| 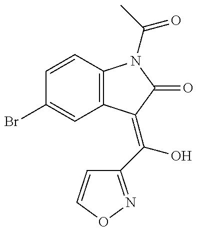 [SMILES: CC(=O)N1C(=O)/C(=C(\O)c2ccon2)c2cc(Br)ccc21] | (Z)-1-acetyl-5-bromo-3- (hydroxy(isoxazol-3- yl)methylene)indolin-2-one | 349.00 | ¹H NMR (400 MHz, DMSO- d₆) δ 8.77 (d, J = 1.7 Hz, 1H), 8.15 (s, 1H), 7.94 (d, J = 8.5 Hz, 1H), 6.98 (dd, J = 8.5, 2.3 Hz, 1H), 6.52 (d, J = 1.7 Hz, 1H), 2.48 (s, 3H). |
| 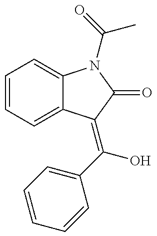 [SMILES: CC(=O)N1C(=O)/C(=C(\O)c2ccccc2)c2ccccc21] | (Z)-1-acetyl-3- (hydroxy(phenyl) methylene) indolin-2-one | 280.10 | ¹H NMR (400 MHz, CDCl₃) δ 8.32-8.27 (m, 1H), 7.66-7.62 (m, 2H), 7.53-7.49 (m, 3H), 7.10-7.05 (m, 1H), 6.95-6.90 (m, 1H), 6.87-6.83 (m, 1H), 2.79 (s, 3H). |
| 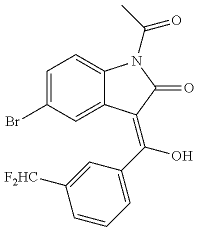 [SMILES: CC(=O)N1C(=O)/C(=C(\O)c2cccc(C(F)F)c2)c2cc(Br)ccc21] | (Z)-1-acetyl-5-bromo-3-((3- (difluoromethyl)phenyl) (hydroxy)methylene)indolin- 2-one | 407.95 | ¹H NMR (400 MHz, DMSO- d₆) δ 8.06 (d, J = 8.6 Hz, 1H), 7.98 (s, 1H), 7.78-7.73 (m, 3H), 7.66 (t, J = 8.0 Hz, 1H), 7.37 (dd, J = 8.6, 2.3 Hz, 1H), 7.13 (t, J = 55.7 Hz, 1H), 2.48 (s, 3H). |
| 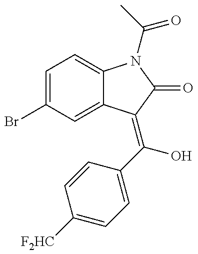 [SMILES: CC(=O)N1C(=O)/C(=C(\O)c2ccc(C(F)F)cc2)c2cc(Br)ccc21] | (Z)-1-acetyl-5-bromo-3- ((4- (difluoromethyl)phenyl) (hydroxy)methylene)indolin- 2-one | 407.85 | ¹H NMR (400 MHz, DMSO- d₆) δ 8.16 (s, 1H), 7.97 (d, J = 8.5 Hz, 1H), 7.61 (d, J = 7.9 Hz, 2H), 7.55 (d, J = 7.9 Hz, 2H), 7.06 (t, J = 56.0 Hz, 1H), 7.04 (dd, J = 8.6, 2.4 Hz, 1H), 2.47 (s, 3H). |
| 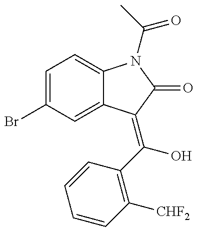 [SMILES: CC(=O)N1C(=O)/C(=C(\O)c2ccccc2C(F)F)c2cc(Br)ccc21] | (Z)-1-acetyl-5-bromo-3- ((2- (difluoromethyl)phenyl) (hydroxy)methylene) indolin- 2-one | 407.80 | ¹H NMR (400 MHz, CDCl₃) 813.54 (s, 1H), 8.17 (d, J = 8.7 Hz, 1H), 7.90 (d, J = 7.8 Hz, 1H), 7.76 (t, J = 7.6 Hz, 1H), 7.69 (t, J = 7.6 Hz, 1H), 7.58 (d, J = 7.6 Hz, 1H), 7.32 (dd, J = 8.7, 2.0 Hz, 1H), 6.91 (t, J = 55.4 Hz, 1H), 6.54 (d, J = 2.0 Hz, 1H), 2.80 (s, 3H). |
| 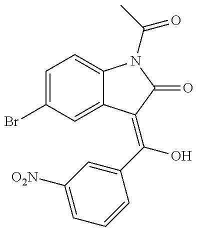 [SMILES: CC(=O)N1C(=O)/C(=C(\O)c2cccc([N+](=O)[O-])c2)c2cc(Br)ccc21] | (Z)-1-acetyl-5-bromo-3- (hydroxy(3- nitrophenyl)methylene) indolin-2-one | 402.95 | ¹H NMR (400 MHz, DMSO- d₆) δ 8.33 (t, J = 1.9 Hz, 1H), 8.29-8.24 (m, 1H), 8.17 (d, J = 2.3 Hz, 1H), 7.94-8.03 (m, 2H), 7.68 (t, J = 7.9 Hz, 1H), 7.12 (dd, J = 8.6, 2.3 Hz, 1H), 2.47 (s, 3H). |
| 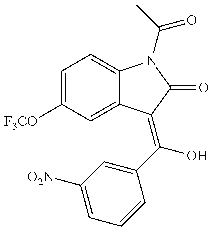 [SMILES: CC(=O)N1C(=O)/C(=C(\O)c2cccc([N+](=O)[O-])c2)c2cc(OC(F)(F)F)ccc21] | (Z)-1-acetyl-3-(hydroxy(3- nitrophenyl)methylene)-5- (trifluoromethoxy)indolin- 2-one | 409.00 | ¹H NMR (400 MHz, CDCl₃) δ 8.63 (t, J = 2.0 Hz, 1H), 8.53- 8.49 (m, 1H), 8.36 (d, J = 9.0 Hz, 1H), 8.09 (dt, J = 7.8, 1.3 Hz, 1H), 7.80 (t, J = 8.0 Hz, 1H), 7.13-7.09 (m, 1H), 6.80 (dd, J = 2.3, 1.1 Hz, 1H), 2.82 (s, 3H). |
| 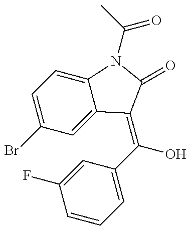 [SMILES: CC(=O)N1C(=O)/C(=C(\O)c2cccc(F)c2)c2cc(Br)ccc21] | (Z)-1-acetyl-5-bromo-3- ((3- fluorophenyl)(hydroxy) methylene)indolin-2-one | 376.00 | ¹H NMR (400 MHz, DMSO- d₆) δ 8.12 (s, 1H), 7.97 (d, J = 8.5 Hz, 1H), 7.44-7.36 (m, 1H), 7.36-7.24 (m, 2H), 7.20 (t, J = 8.8 Hz, 1H), 7.04 (dd, J = 8.5, 2.3 Hz, 1H), 4.32-3.93 (m, 1H), 2.49 (s, 3H). |
| 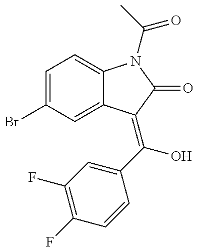 [SMILES: CC(=O)N1C(=O)/C(=C(\O)c2ccc(F)c(F)c2)c2cc(Br)ccc21] | (Z)-1-acetyl-5-bromo-3- ((3,4- difluorophenyl)(hydroxy) methylene)indolin-2-one | 394.00 | ¹H NMR (400 MHz, DMSO- d₆) δ 8.06-8.01 (m, 2H), 7.75- 7.67(m, 1H), 7.61-7.51 (m, 1H), 7.47-7.41 (m, 1H), 7.33 (dd, J = 8.6, 2.3 Hz, 1H), 2.48 (s, 3H). |

### Preparation of Key Intermediates A and B

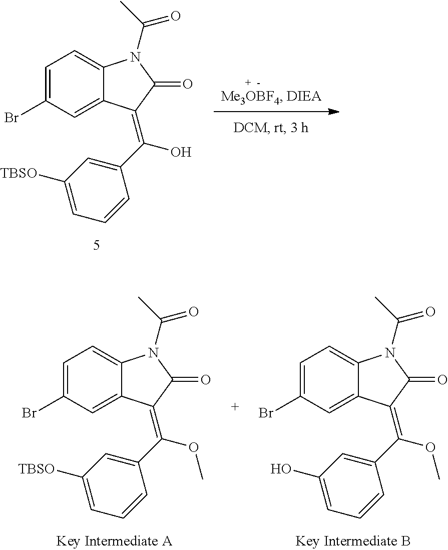 [SMILES: CC(=O)N1C(=O)/C(=C(\O)c2cccc(O[Si](C)(C)C(C)(C)C)c2)c2cc(Br)ccc21 ; "Me3OBF4, DIEA" ; CO/C(=C1\C(=O)N(C(C)=O)c2ccc(Br)cc21)c1cccc(O[Si](C)(C)C(C)(C)C)c1 ; CO/C(=C1\C(=O)N(C(C)=O)c2ccc(Br)cc21)c1cccc(O)c1]

To a stirred mixture of 1-acetyl-5-bromo-3-((3-((tert-butyldimethylsilyl)oxy)phenyl) (hydroxy)methylene)indolin-2-one (6 g, 12.28 mmol) and DIEA (6.35 g, 49.17 mmol) in DCM (150 mL) was added trimethyloxonium tetrafluoroborate (10.90 g, 73.70 mmol) at room temperature under nitrogen atmosphere. The resulting mixture was stirred for 3 h at room temperature under nitrogen atmosphere. The resulting mixture was diluted with DCM (1 L). The resulting mixture was washed with water (150 mL) and brine (2×200 mL). The organic layer was dried over anhydrous Na₂SO₄. After filtration, the filtrate was concentrated under reduced pressure. The residue was purified by silica gel column chromatography, eluted with petroleum ether/EtOAc (100:1 to 3:1) to afford (Z)-1-acetyl-5-bromo-3-((3-((tert-butyldimethylsilyl)oxy) phenyl)(methoxy)methylene)indolin-2-one (3.67 g, 60%) as a yellow solid: ¹H NMR (400 MHz, DMSO-d₆) δ 8.08 (d, J=8.7 Hz, 1H), 8.04 (d, J=2.1 Hz, 1H), 7.48-7.44 (m, 2H), 7.06-7.02 (m, 2H), 6.96-6.93 (m, 1H), 3.75 (s, 3H), 2.42 (s, 3H), 0.96 (s, 9H), 0.21 (s, 6H); LC/MS (ESI, m/z): [(M+1)]⁺=502.15. and (Z)-1-acetyl-5-bromo-3-((3-hydroxyphenyl)(methoxy)methylene) indolin-2-one (900 mg, 19%) as a yellow solid: ¹H NMR (400 MHz, DMSO-d₆) δ 9.76 (s, 1H), 8.08 (d, J=8.7 Hz, 1H), 8.03 (d, J=2.2 Hz, 1H), 7.46 (dd, J=8.6, 2.2 Hz, 1H), 7.35 (t, J=7.8 Hz, 1H), 6.98-6.92 (m, 1H), 6.84 (dt, J=7.6, 1.2 Hz, 1H), 6.79 (dd, J=2.5, 1.5 Hz, 1H), 3.75 (s, 3H), 2.43 (s, 3H); LC/MS (ESI, m/z): [(M+1)]⁺=388.00.

The following intermediates were prepared using the synthetic procedures of the Key Intermediates A and B described above.

| | | MS: | |
|---|---|---|---|
| Structure | Name | [(M + 1)]⁺ | ¹H NMR |
| 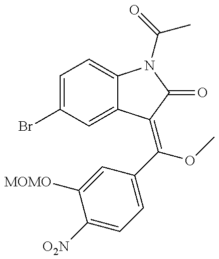 | (Z)-1-acetyl-5-bromo- 3-(methoxy(3- (methoxymethoxy)-4- nitrophenyl)methylene) indolin-2-one | 476.90 | ¹H NMR (400 MHz, DMSO-d₆) δ 8.11-8.02 (m, 3H), 7.58 (d, J = 1.6 Hz, 1H), 7.50 (dd, J = 8.7, 2.2 Hz, 1H), 7.32 (dd, J = 8.2, 1.6 Hz, 1H), 5.40 (s, 2H), 3.81 (s, 3H), 3.43 (s, 3H), 2.44 (s, 3H). |
| 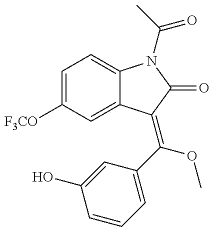 [SMILES: CO/C(=C1\C(=O)N(C(C)=O)c2ccc(OC(F)(F)F)cc21)c1cccc(O)c1] | (Z)-1-acetyl-3-((3- ((tert-butyldimethyl silyl)oxy)phenyl) (methoxy)methylene)- 5-(trifluoromethoxy) indolin-2-one | 394.15 | ¹H NMR (400 MHz, DMSO-d₆) δ 9.77 (s, 1H), 8.22 (d, J = 8.9 Hz, 1H), 7.83 (d, J = 2.6 Hz, 1H), 7.36 (t, J = 7.9 Hz, 1H), 7.31-7.26 (m, 1H), 6.98-6.94 (m, 1H), 6.88-6.84 (m, 1H), 6.81 (t, J = 2.0 Hz, 1H), 3.75 (s, 3H), 2.45 (s, 3H). |
| 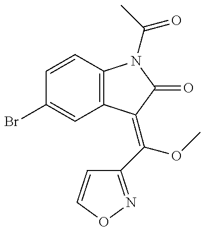 [SMILES: CO/C(=C1\C(=O)N(C(C)=O)c2ccc(Br)cc21)c1ccon1] | (Z)-1-acetyl-5-bromo- 3-(isoxazol-3-yl (methoxy)methylene) indolin-2-one | 362.90 | ¹H NMR (400 MHz, DMSO-d₆) δ 7.86 (d, J = 1.6 Hz, 1H), 7.33 (d, J = 8.7 Hz, 1H), 7.22 (d, J = 2.2 Hz, 1H), 6.60 (dd, J = 8.7, 2.2 Hz, 1H), 5.74 (d, J = 1.6 Hz, 1H), 3.05 (s, 3H), 1.74 (s, 3H). |
| 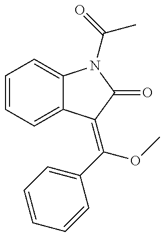 [SMILES: CO/C(=C1\C(=O)N(C(C)=O)c2ccccc21)c1ccccc1] | (Z)-1-acetyl-3- (methoxy(phenyl) methylene)indolin-2- one | 294.00 | ¹H NMR (400 MHz, CDCl₃) δ 8.31-8.26 (m, 1H), 7.98 (dd, J = 7.5, 1.6 Hz, 1H), 7.57-7.55 (m, 2H), 7.41-7.36 (m, 2H), 7.32-7.18 (m, 3H), 3.71 (s, 3H), 2.56 (s, 3H). |
| 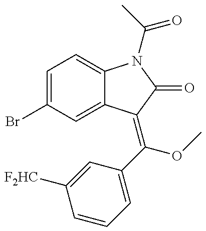 [SMILES: CO/C(=C1\C(=O)N(C(C)=O)c2ccc(Br)cc21)c1cccc(C(F)F)c1] | (Z)-1-acetyl-5-bromo- 3-((3-(difluoromethyl) phenyl)(methoxy) methylene)indolin- 2-one | 422.00 | ¹H NMR (400 MHz, DMSO-d₆) δ 8.08 (d, J = 8.7 Hz, 1H), 8.06 (d, J = 2.2 Hz, 1H), 7.79 (d, J = 7.5 Hz, 1H), 7.76-7.65 (m, 3H), 7.48 (dd, J = 8.7, 2.2 Hz, 1H), 7.13 (t, J = 55.7 Hz, 1H), 3.76 (s, 3H), 2.42 (s, 3H). |
| 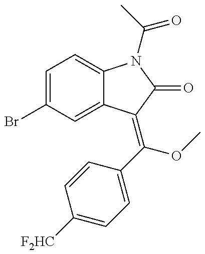 [SMILES: CO/C(=C1\C(=O)N(C(C)=O)c2ccc(Br)cc21)c1ccc(C(F)F)cc1] | (Z)-1-acetyl-5-bromo- 3-((4-(difluoromethyl) phenyl)(methoxy) methylene) indolin-2-one | 422.05 | ¹H NMR (400 MHz, DMSO-d₆) δ 8.09 (d, J = 8.7 Hz, 1H), 8.06 (d, J = 2.1 Hz, 1H), 7.76 (d, J = 8.0 Hz, 2H), 7.64 (d, J = 8.0 Hz, 2H), 7.49 (dd, J = 8.6, 2.2 Hz, 1H), 7.16 (t, J = 55.7 Hz, 1H), 3.75 (s, 3H), 2.41 (s, 3H). |
| 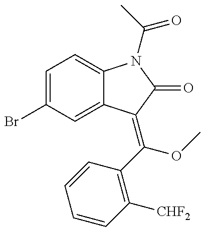 [SMILES: CO/C(=C1\C(=O)N(C(C)=O)c2ccc(Br)cc21)c1ccccc1C(F)F] | (Z)-1-acetyl-5-bromo- 3-((2-(difluoromethyl) phenyl)(methoxy) methylene)indolin- 2-one | 422.25 | ¹H NMR (400 MHz, CDCl₃) δ 8.17 (d, J = 8.7 Hz, 1H), 8.10 (d, J = 2.2 Hz, 1H), 7.84-7.77 (m, 1H), 7.71-7.65 (m, 2H), 7.41 (dd, J = 8.7, 2.2 Hz, 1H), 7.35-7.28 (m, 1H), 6.67 (t, J = 55.4 Hz, 1H), 3.73 (s, 3H), 2.51 (s, 3H). |
| 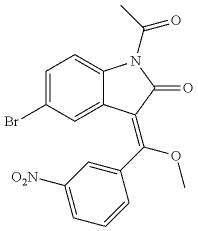 [SMILES: CO/C(=C1\C(=O)N(C(C)=O)c2ccc(Br)cc21)c1cccc([N+](=O)[O-])c1] | (Z)-1-acetyl-5-bromo- 3-(methoxy(3- nitrophenyl) methylene) indolin-2-one | 416.95 | ¹H NMR (400 MHz, DMSO-d₆) δ 8.46-8.41 (m, 2H), 8.09 (d, J = 8.7 Hz, 1H), 8.06 (d, J = 2.2 Hz, 1H), 7.98 (dt, J = 7.6, 1.4 Hz, 1H), 7.88 (dt, J = 7.7, 1.0 Hz, 1H), 7.50 (dd, J = 8.7, 2.2 Hz, 1H), 3.79 (s, 3H), 2.40 (s, 3H). |
| 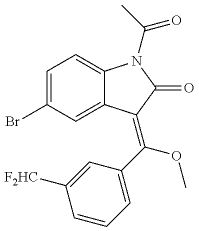 [SMILES: CO/C(=C1\C(=O)N(C(C)=O)c2ccc(Br)cc21)c1cccc(C(F)F)c1] | (Z)-1-acetyl-5-bromo- 3-((3-(difluoromethyl) phenyl)(methoxy) methylene)indolin- 2-one | 422.05 | ¹H NMR (400 MHz, DMSO-d₆) δ 8.11-8.03 (m, 2H), 7.79 (d, J = 7.7 Hz, 1H), 7.75-7.64 (m, 3H), 7.48 (dd, J = 8.7, 2.2 Hz, 1H), 7.13 (t, J = 55.7 Hz, 1H), 3.74 (s, 3H), 2.41 (s, 3H). |
| 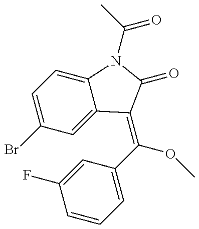 [SMILES: CO/C(=C1\C(=O)N(C(C)=O)c2ccc(Br)cc21)c1cccc(F)c1] | (Z)-1-acetyl-5-bromo- 3-((3-fluorophenyl) (methoxy)methylene) indolin-2-one | 389.85 | ¹H NMR (400 MHz, CDCl₃) δ 8.17 (d, J = 8.7 Hz, 1H), 8.06 (d, J = 2.1 Hz, 1H), 7.55 (td, J = 8.0, 5.6 Hz, 1H), 7.40 (dd, J = 8.7, 2.2 Hz, 1H), 7.30-7.24 (m, 1H), 7.16 (dd, J = 7.6, 1.4 Hz, 1H), 7.09 (dt, J = 8.8, 1.9 Hz, 1H), 3.76 (d, J = 1.0 Hz, 3H), 2.55 (d, J = 1.0 Hz, 3H). |
| 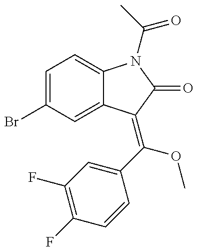 [SMILES: CO/C(=C1\C(=O)N(C(C)=O)c2ccc(Br)cc21)c1ccc(F)c(F)c1] | (Z)-1-acetyl-5-bromo- 3-((3,4-difluoro phenyl)(methoxy) methylene)indolin- 2-one | 408.10 | ¹H NMR (400 MHz, DMSO-d₆) δ 8.07 (d, J = 8.7 Hz, 1H), 8.02 (d, J = 2.2 Hz, 1H), 7.75-7.61 (m, 2H), 7.47 (dd, J = 8.6, 2.2, Hz, 1H), 7.41-7.37 (m, 1H), 3.79 (s, 3H), 2.43 (s, 3H). |

The following Indolinone Intermediates with NH₂ and MeNH₂ were prepared according to the General Synthetic Procedure of Scheme 1.

MS:

| Structure | Name | [(M + 1)]⁺ | ¹H NMR |
|---|---|---|---|
| 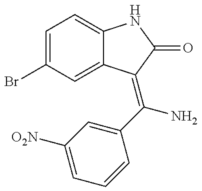 [SMILES: N/C(=C1\C(=O)Nc2ccc(Br)cc21)c1cccc([N+](=O)[O-])c1] | (Z)-3-(amino(3- nitrophenyl) methylene)-5- bromoindolin-2- one | 359.90. | ¹H NMR (400 MHz, DMSO- d₆) δ 10.60 (s, 1H), 9.53 (d, J = 4.1 Hz, 1H), 8.51 (dd, J = 8.2, 2.4, 1H), 8.47 (d, J = 4.2 Hz, 1H), 8.37 (t, J = 1.9 Hz, 1H), 8.05 (dt, J = 7.7, 1.4 Hz, 1H), 7.93 (t, J = 8.0 Hz, 1H), 7.01 (dd, J = 8.2, 2.0 Hz, 1H), 6.77 (d, J = 8.2 Hz, 1H), 6.01 (d, J = 1.9 Hz, 1H). |
| 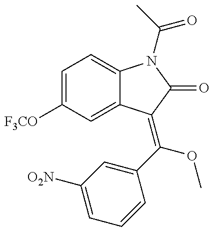 [SMILES: CO/C(=C1\C(=O)N(C(C)=O)c2ccc(OC(F)(F)F)cc21)c1cccc([N+](=O)[O-])c1] | (Z)-1-acetyl-3- (methoxy(3- nitrophenyl) methylene)-5- (trifluoromethoxy) indolin-2-one | 423.10 | ¹H NMR (400 MHz, CDCl₃) δ 8.47-8.43 (m, 1H), 8.33-8.28 (m, 2H), 7.83-7.72 (m, 3H), 7.20-7.16 (m, 1H), 3.79 (s, 3H), 2.53 (s, 3H). |

### Preparation of 1-Acetyl-5-(trifluoromethoxy)indolin-2-one

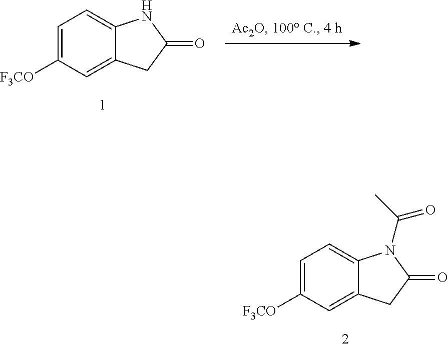 [SMILES: O=C1Cc2cc(OC(F)(F)F)ccc2N1 ; "Ac2O, 100° C., 4 h" ; CC(=O)N1C(=O)Cc2cc(OC(F)(F)F)ccc21]

A solution of 5-(trifluoromethoxy)-1,3-dihydroindol-2-one (2.30 g, 10.59 mmol) in Ac₂O (46 mL) was stirred for 4 h at 100° C. under nitrogen atmosphere. The resulting mixture was concentrated under reduced pressure. The residue was purified by reverse phase flash chromatography with the following conditions: Column: Spherical C18, 20-40 μm, 120 g; Eluent A: Water (plus 10 mM NH₄HCO₃); Eluent B: ACN; Flow rate: 50 mL/min; Gradient: 70% B-95% B in 20 min; Detector: UV 254 nm. The fractions containing desired product were collected at 70% B and concentrated under reduced pressure to afford 1-acetyl-5-(trifluoromethoxy) indolin-2-one (1.75 g, 64%) as a yellow solid: H NMR (400 MHz, DMSO-d₆) δ 8.15 (d, J=8.9 Hz, 1H), 7.40 (d, J=2.2, 1H), 7.33 (dd, J=9.0, 2.9 Hz, 1H), 3.87 (s, 2H), 2.56 (s, 3H); LC/MS (ESI, m/z): [(M−1)]⁻=258.05.

### Preparation of 1-Acetyl-3H-indol-2-one

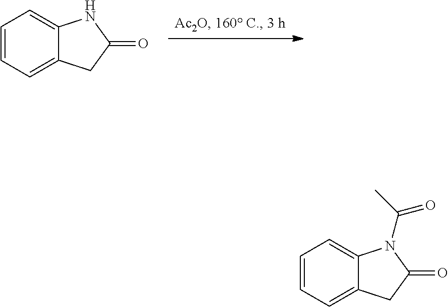

A solution of oxindole (8 g, 60.08 mmol) in acetic anhydride (80 mL, 846.32 mmol) was stirred for 3 h at 160° C. under nitrogen atmosphere. The resulting mixture was concentrated under reduced pressure. The residue was purified by trituration with petroleum ether/CH₂Cl₂ (3/1, 50 mL) to afford 1-acetyl-3H-indol-2-one (8 g, 76%) as an off-white solid: ¹H NMR (400 MHz, CDCl₃) δ 8.24 (d, J=8.2 Hz, 1H), 7.39-7.26 (m, 2H), 7.24-7.18 (m, 1H), 3.74 (s, 2H), 2.70 (s, 3H); LC/MS (ESI, m/z): [(M+1)]⁺=176.20.

### Preparation of Key Intermediate C (3,5-Dibromoindolin-2-one)

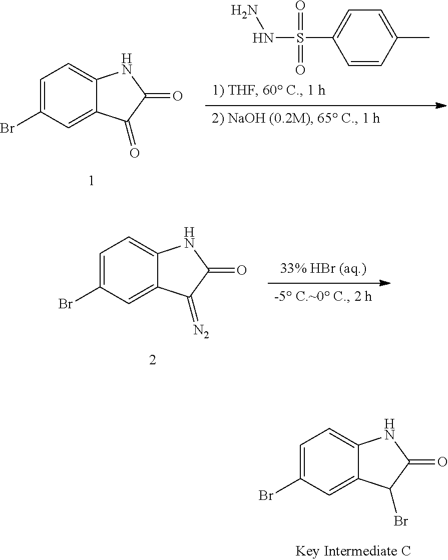

### Preparation of 5-Bromo-3-diazoindolin-2-one

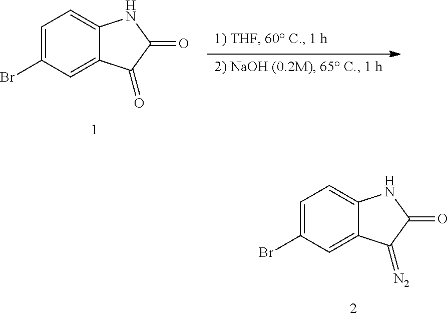

To a stirred solution of 5-bromoisatin (10 g, 44.24 mmol) in THF (65 mL) was added 4-toluenesulfonyl hydrazide (8.65 g, 46.45 mmol) in portions at room temperature under nitrogen atmosphere. The resulting mixture was stirred for 1 h at 60° C. under nitrogen atmosphere. After cooling down to ambient temperature, the precipitated solids were collected by filtration and washed with THF (3×30 mL). The filter cake was added to 0.2 M NaOH (aq.) (100 mL) at room temperature. The resulting mixture was stirred for additional 1 h at 65° C. The mixture was neutralized to pH 7 with dry ice. The precipitated solids were collected by filtration and washed with H₂O (3×100 mL). The filter cake was dried under infrared light to afford 5-bromo-3-diazoindolin-2-one (10 g, 94%) as a yellow solid which was used in the next step directly without further purification: ¹H NMR (400 MHz, DMSO-d₆) δ 10.79 (s, 1H), 7.70 (d, J=2.0 Hz, 1H), 7.24 (dd, J=8.3, 2.0 Hz, 1H), 6.85 (d, J=8.3 Hz, 1H); LC/MS (ESI, m/z): [(M+1)]⁺=237.75.

### Preparation of Key Intermediate C (3,5-dibromoindolin-2-one)

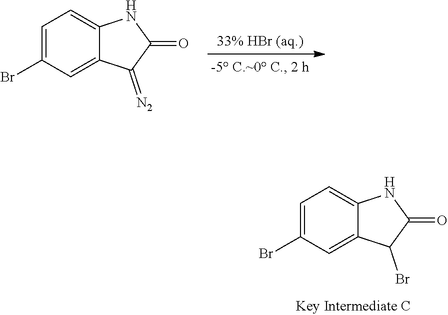

To a cooled solution of 33% HBr (aq.) (80 mL) was added 5-bromo-3-(-lambda5-diazynylidene)-1H-indol-2-one (8.80 g, 36.97 mmol) in small potions at −5° C. The resulting mixture was stirred for 2 h at 0° C. under nitrogen atmosphere. The resulting mixture was diluted with H₂O (100 mL). The resulting mixture was extracted with EtOAc (3×100 mL). The combined organic layers were washed with brine (3×50 mL), dried over anhydrous Na₂SO₄. After filtration, the filtrate was concentrated under reduced pressure. The residue was purified by silica gel column chromatography, eluted with petroleum ether/EtOAc (5:1 to 1:1) to afford crude 3,5-dibromoindolin-2-one (10 g, 93%) as a brown solid which was used in the next step without further purification: LC/MS (ESI, m/z): [(M+1)]⁺=291.80.

### Preparation of Enamine Compounds

### General Procedure A

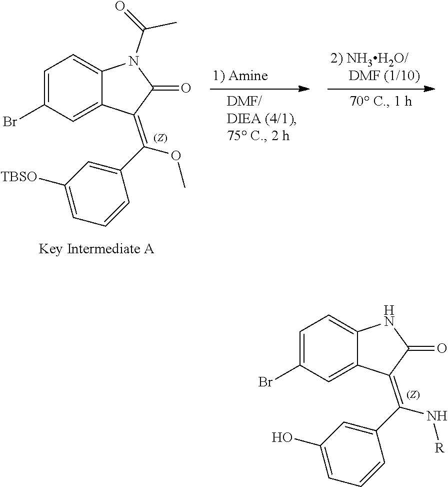

A solution of (Z)-1-acetyl-5-bromo-3-((3-((tert-butyldimethylsilyl)oxy)phenyl) (methoxy)methylene)indolin-2-one (65 mg, 0.13 mmol) and Amine (0.39 mmol) in DMF (2 mL) and DIEA (0.5 mL) was stirred for 2 h at 75° C. under nitrogen atmosphere followed by the addition of NH₃·H₂O (aq.) (0.25 mL). After stirring for additional 1 h at 70° C. The resulting mixture was purified by reversed phase flash chromatography. The fractions containing desired product were collected and concentrated under reduced pressure to afford desired product.

### General Procedure B

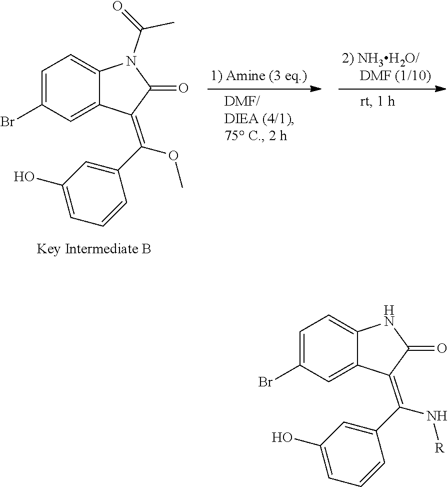

A solution of (Z)-1-acetyl-5-bromo-3-((3-hydroxyphenyl)(methoxy)methylene)indolin-2-one (50 mg, 0.13 mmol) and Amine (0.39 mmol) in DMF (2 mL) and DIEA (0.5 mL) was stirred for 2 h at 75° C. under nitrogen atmosphere. After cooling down to ambient temperature, NH₃·H₂O (aq.) (0.25 mL) was added. The resulting mixture was stirred for additional 1 h at ambient temperature. The resulting mixture was purified by reversed phase flash chromatography. The fractions containing desired product were collected and concentrated under reduced pressure to afford desired product.

The following exemplary compounds were prepared using General Procedure A or B

| Ex. | | | MS: | |
|---|---|---|---|---|
| No. | Structure | Name | [(M + 1)]+ | ¹H NMR |
| 1 | 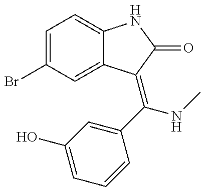 [SMILES: CN/C(=C1\C(=O)Nc2ccc(Br)cc21)c1cccc(O)c1] | (Z)-5-bromo-3-((3- hydroxyphenyl) (methylamino) methylene) indolin-2-one | 345.05 | ¹H NMR (400 MHz, DMSO-d₆) δ 10.48 (s, 1H), 10.10 (q, J = 5.0 Hz, 1H), 9.90 (s, 1H), 7.47 (t, J = 7.9 Hz, 1H), 7.06-7.00 (m, 1H), 6.92 (dd, J = 8.2, 2.0 Hz, 1H), 6.81 (dt, J = 7.5, 1.3 Hz, 1H), 6.76 (t, J = 2.0 Hz, 1H), 6.71 (d, J = 8.2 Hz, 1H), 5.68 (d, J = 2.0 Hz, 1H), 2.80 (d, J = 5.2 Hz, 3H). |
| 2 | 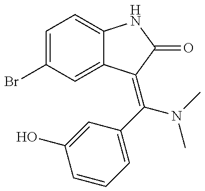 [SMILES: CN(C)/C(=C1\C(=O)Nc2ccc(Br)cc21)c1cccc(O)c1] | (Z)-5-bromo-3- ((dimethylamino)(3- hydroxyphenyl) methylene) indolin-2-one | 359.05 | ¹H NMR (400 MHz, DMSO-d₆) δ 10.11 (s, 1H), 9.79 (s, 1H), 7.40 (t, J = 7.9 Hz, 1H), 7.06- 7.00 (m, 1H), 6.89 (d, J = 7.5 Hz, 1H), 6.84 (dd, J = 8.2, 1.9 Hz, 1H), 6.77 (t, J = 1.9 Hz, 1H), 6.63 (d, J = 8.2 Hz, 1H), 5.51 (d, J = 2.0 Hz, 1H), 3.16 (s, 6H). |
| 3 | 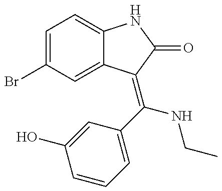 [SMILES: CCN/C(=C1\C(=O)Nc2ccc(Br)cc21)c1cccc(O)c1] | (Z)-5-bromo-3- ((ethylamino)(3- hydroxyphenyl) methylene)indolin- 2-one | 359.05 | ¹H NMR (400 MHz, DMSO-d₆) δ 10.52 (s, 1H), 10.20 (t, J = 6.2 Hz, 1H), 9.88 (br, 1H), 7.46 (t, J = 7.9 Hz, 1H), 7.07-6.99 (m, 1H), 6.92 (dd, J = 8.2, 2.0 Hz, 1H), 6.82 (dt, J = 7.4, 1.3 Hz, 1H), 6.76 (t, J = 2.0 Hz, 1H), 6.71 (d, J = 8.1 Hz, 1H), 5.63 (d, J = 2.0 Hz, 1H), 3.18-3.09 (m, 2H), 1.10 (t, J = 7.2 Hz, 3H). |
| 4 | 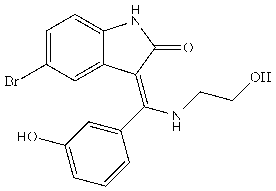 [SMILES: O=C1Nc2ccc(Br)cc2/C1=C(/NCCO)c1cccc(O)c1] | (Z)-5-bromo-3-(((2- hydroxyethyl)amino) (3-hydroxyphenyl) methylene)indolin-2- one | 375.00 | ¹H NMR (400 MHz, DMSO-d₆) δ 10.50 (s, 1H), 10.34 (t, J = 6.2 Hz, 1H), 9.88 (s, 1H), 7.46 (t, J = 7.9 Hz, 1H), 7.05-6.99 (m, 1H), 6.92 (dd, J = 8.2, 2.0 Hz, 1H), 6.81 (dt, J = 7.4, 1.2 Hz, 1H), 6.76 (t, J = 1.9 Hz, 1H), 6.71 (d, J = 8.2 Hz, 1H), 5.62 (d, J = 2.0 Hz, 1H), 4.93 (t, J = 5.1 Hz, 1H), 3.47 (q, J = 5.4 Hz, 2H), 3.16 (q, J = 5.7 Hz, 2H). |
| 5 | 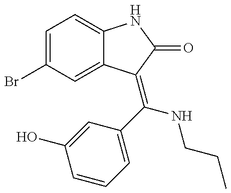 [SMILES: CCCN/C(=C1\C(=O)Nc2ccc(Br)cc21)c1cccc(O)c1] | (Z)-5-bromo-3-((3- hydroxyphenyl) (propylamino) methylene) indolin-2-one | 373.05 | ¹H NMR (400 MHz, DMSO-d₆) δ 10.53 (s, 1H), 10.30 (t, J = 6.2 Hz, 1H), 9.94 (s, 1H), 7.46 (t, J = 7.8 Hz, 1H), 7.03 (dd, J = 8.2, 2.5 Hz, 1H), 6.92 (dd, J = 8.2, 2.0 Hz, 1H), 6.81 (dt, J = 7.4, 1.2 Hz, 1H), 6.75 (t, J = 2.0 Hz, 1H), 6.72 (d, J = 8.1 Hz, 1H), 5.65 (d, J = 2.0 Hz, 1H), 3.07 (q, J = 6.7 Hz, 2H), 1.56-1.44 (m, 2H), 0.85 (t, J = 7.4 Hz, 3H). |
| 6 | 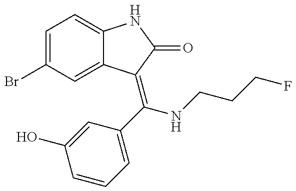 [SMILES: O=C1Nc2ccc(Br)cc2/C1=C(/NCCCF)c1cccc(O)c1] | (Z)-5-bromo-3-(((3- fluoropropyl)amino) (3-hydroxyphenyl) methylene)indolin-2- one | 391.05 | ¹H NMR (400 MHz, DMSO-d₆) δ 10.56 (s, 1H), 10.26 (t, J = 6.2 Hz, 1H), 9.93 (s, 1H), 7.47 (t, J = 7.8 Hz, 1H), 7.04 (ddd, J = 8.2, 2.5, 1.0 Hz, 1H), 6.94 (dd, J = 8.2, 2.0 Hz, 1H), 6.83 (dt, J = 7.5, 1.2 Hz, 1H), 6.77 (t, J = 2.0 Hz, 1H), 6.72 (d, J = 8.2 Hz, 1H), 5.63 (d, J = 2.0 Hz, 1H), 4.53 (t, J = 5.7 Hz, 1H), 4.41 (t, J = 5.7 Hz, 1H), 3.23 (q, J = 6.7 Hz, 2H), 1.95-1.80 (m, 2H). |
| 7 | 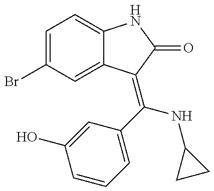 [SMILES: O=C1Nc2ccc(Br)cc2/C1=C(/NC1CC1)c1cccc(O)c1] | (Z)-5-bromo-3- ((cyclopropylamino) (3-hydroxyphenyl) methylene)indolin-2- one | 371.05 | ¹H NMR (400 MHz, DMSO-d₆) δ 10.56 (s, 1H), 10.15 (d, J = 4.5 Hz, 1H), 9.85 (s, 1H), 7.46 (t, J = 7.9 Hz, 1H), 7.02 (dd, J = 8.3, 2.5, Hz, 1H), 6.95 (dd, J = 8.2, 2.0 Hz, 1H), 6.89 (dt, J = 7.6, 1.2 Hz, 1H), 6.84 (dd, J = 2.5, 1.5 Hz, 1H), 6.72 (d, J = 8.2 Hz, 1H), 5.76 (d, J = 2.0 Hz, 1H), 2.62-2.54 (m, 1H), 0.63-0.58 (m, 4H). |
| 8 | 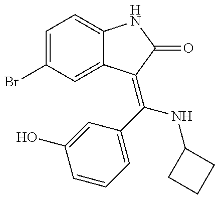 [SMILES: O=C1Nc2ccc(Br)cc2/C1=C(/NC1CCC1)c1cccc(O)c1] | (Z)-5-bromo-3- ((cyclobutylamino) (3-hydroxyphenyl) methylene)indolin-2- one | 385.05 | ¹H NMR (400 MHz, DMSO-d₆) δ 10.58 (s, 1H), 10.38 (d, J = 8.6 Hz, 1H), 9.91 (s, 1H), 7.46 (t, J = 7.9 Hz, 1H), 7.04 (dd, J = 8.2, 2.5 Hz, 1H), 6.95 (dd, J = 8.2, 2.0 Hz, 1H), 6.80-6.76 (m, 1H), 6.74-6.70 (m, 2H), 5.70 (d, J = 2.0 Hz, 1H), 3.78-3.66 (m, 1H), 2.17-1.96 (m, 4H), 1.70-1.41 (m, 2H). |
| 9 | 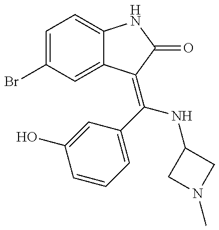 [SMILES: CN1CC(N/C(=C2\C(=O)Nc3ccc(Br)cc32)c2cccc(O)c2)C1] | (Z)-5-bromo-3-((3- hydroxyphenyl)((1- methylazetidin-3- yl)amino)methylene) indolin-2-one | 400.00 | ¹H NMR (400 MHz, DMSO-d₆) δ 10.64 (s, 1H), 10.46 (d, J = 8.7 Hz, 1H), 9.95 (s, 1H), 7.45 (t, J = 7.9 Hz, 1H), 7.04 (dd, J = 8.2, 2.4 Hz, 1H), 6.96 (dd, J = 8.2, 2.1 Hz, 1H), 6.80-6.69 (m, 3H), 5.72 (d, J = 2.0 Hz, 1H),3.83- 3.73 (m, 1H), 3.44-3.35 (m, 3H), 2.99-2.90 (m, 1H), 2.22 (s, 3H). |
| 10 | 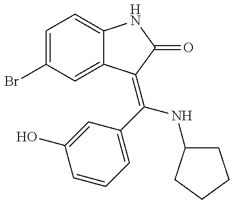 [SMILES: O=C1Nc2ccc(Br)cc2/C1=C(/NC1CCCC1)c1cccc(O)c1] | (Z)-5-bromo-3- ((cyclopentylamino) (3-hydroxyphenyl) methylene)indolin-2- one | 399.10 | ¹H NMR (400 MHz, DMSO-d₆) δ 10.55 (s, 1H), 10.38 (d, J = 9.2 Hz, 1H), 9.91 (s, 1H), 7.46 (t, J = 7.8 Hz, 1H), 7.03-7.05 (m, 1H), 6.92 (dd, J = 8.2, 2.0 Hz, 1H), 6.85-6.81 (m, 1H), 6.80- 6.75 (m, 1H), 6.72 (d, J = 8.2 Hz, 1H), 5.63 (d, J = 2.0 Hz, 1H), 3.69-3.59 (m, 1H), 1.82- 1.76 (m, 2H), 1.74-1.63 (m, 2H), 1.57-1.45 (m, 4H). |
| 11 | 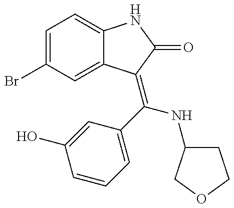 [SMILES: O=C1Nc2ccc(Br)cc2/C1=C(/NC1CCOC1)c1cccc(O)c1] | (Z)-5-bromo-3-((3- hydroxyphenyl) ((tetrahydrofuran- 3-yl) amino)methylene) indolin-2-one | 401.05 | ¹H NMR (400 MHz, DMSO-d₆) δ 10.59 (s, 1H), 10.43 (d, J = 9.2 Hz, 1H), 9.92 (d, J = 3.5 Hz, 1H), 7.53-7.45 (m, 1H), 7.07- 7.03 (m, 1H), 6.95 (dd, J = 8.2, 2.0 Hz, 1H), 6.90-6.75 (m, 2H), 6.72 (d, J = 8.2 Hz, 1H), 5.67 (d, J = 1.9 Hz, 1H), 3.97-3.85 (m, 2H), 3.69-3.58 (m, 3H), 2.20-2.04 (m, 1H), 1.92-1.80 (m, 1H). |
| 12 | 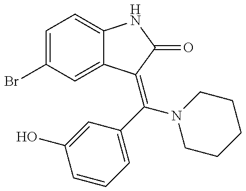 [SMILES: O=C1Nc2ccc(Br)cc2/C1=C(\c1cccc(O)c1)N1CCCCC1] | (Z)-5-bromo-3-((3- hydroxyphenyl) (piperidin-1- yl)methylene) indolin-2-one | 399.05 | ¹H NMR (400 MHz, DMSO-d₆) δ 10.10 (s, 1H), 9.79 (s, 1H), 7.39 (t, J = 7.9 Hz, 1H), 7.06- 6.99 (m, 1H), 6.89 (d, J = 7.5 Hz, 1H), 6.83 (dd, J = 8.2, 2.0 Hz, 1H), 6.79 (t, J = 2.0 Hz, 1H), 6.60 (d, J = 8.2 Hz, 1H), 5.50 (d, J = 2.0 Hz, 1H), 3.31 (br, 4H), 1.68 (s, 6H). |
| 13 | 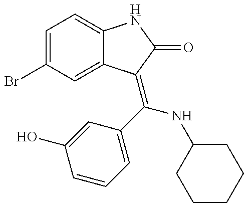 [SMILES: O=C1Nc2ccc(Br)cc2/C1=C(/NC1CCCCC1)c1cccc(O)c1] | (Z)-5-bromo-3- ((cyclohexylamino) (3-hydroxyphenyl) methylene)indolin-2- one | 415.05 | ¹H NMR (400 MHz, DMSO-d₆) δ 10.55 (s, 1H), 10.41 (d, J = 9.6 Hz, 1H), 9.90 (s, 1H), 7.46 (t, J = 7.9 Hz, 1H), 7.03 (dd, J = 8.4, 2.4 Hz, 1H), 6.92 (dd, J = 8.2, 2.0 Hz, 1H), 6.86-6.80 (m, 1H), 6.77 (t, J = 2.0 Hz, 1H), 6.71 (d, J = 8.2 Hz, 1H), 5.60 (d, J = 2.0 Hz, 1H), 3.22-3.10 (m, 1H), 1.77 (d, J = 12.3 Hz, 2H), 1.69- 1.58 (m, 2H), 1.49-1.29 (m, 3H), 1.26-1.13 (m, 3H). |
| 14 | 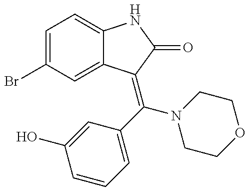 [SMILES: O=C1Nc2ccc(Br)cc2/C1=C(\c1cccc(O)c1)N1CCOCC1] | (Z)-5-bromo-3-((3- hydroxyphenyl) (morpholino) methylene) indolin-2-one | 401.05 | ¹H NMR (400 MHz, DMSO-d₆) δ 10.19 (s, 1H), 9.82 (s, 1H), 7.41 (t, J = 7.8 Hz, 1H), 7.07- 7.02 (m, 1H), 6.96-6.92 (m, 1H), 6.87 (dd, J = 8.2, 2.0 Hz, 1H), 6.84-6.81 (m, 1H), 6.63 (d, J = 8.1 Hz, 1H), 5.52 (d, J = 2.0 Hz, 1H), 3.76 (s, 4H), 3.46 (s, 4H). |
| 15 | 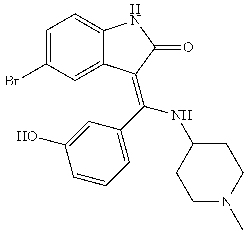 [SMILES: CN1CCC(N/C(=C2\C(=O)Nc3ccc(Br)cc32)c2cccc(O)c2)CC1] | (Z)-5-bromo-3-((3- hydroxyphenyl)((1- methylpiperidin-4- yl)amino)methylene) indolin-2-one | 428.10 | ¹H NMR (400 MHz, DMSO-d₆) δ 10.56 (s, 1H), 10.35 (d, J = 9.5 Hz, 1H), 9.92 (s, 1H), 7.46 (t, J = 7.9 Hz, 1H), 7.07-7.00 (m, 1H), 6.93 (dd, J = 8.2, 2.0 Hz, 1H), 6.84 (d, J = 7.5 Hz, 1H), 6.77 (t, J = 2.0 Hz, 1H), 6.72 (d, J = 8.2 Hz, 1H), 5.60 (d, J = 2.0 Hz, 1H), 3.20-3.08 (m, 1H), 2.64-2.53 (m, 2H), 2.11 (s, 3H), 1.95-1.81 (m, 2H), 1.81-1.70 (m, 2H), 1.58-1.47 (m, 2H). |
| 16 | 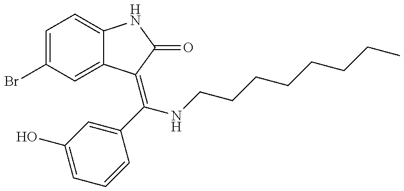 [SMILES: CCCCCCCCN/C(=C1\C(=O)Nc2ccc(Br)cc21)c1cccc(O)c1] | (Z)-5-bromo-3-((3- hydroxyphenyl) (octylamino) methylene) indolin-2-one | 443.10 | ¹H NMR (400 MHz, DMSO-d₆) δ 10.51 (s, 1H), 10.28 (t, J = 6.2 Hz, 1H), 9.89 (s, 1H), 7.46 (t, J = 7.8 Hz, 1H), 7.05-7.00 (m, 1H), 6.92 (dd, J = 8.2, 2.0 Hz, 1H), 6.80 (dt, J = 7.4, 1.3 Hz, 1H), 6.77-6.68 (m, 2H), 5.65 (d, J = 2.0 Hz, 1H), 3.15-3.04 (m, 2H), 1.53-1.41 (m, 2H), 1.31- 1.13(m, 10H), 0.84 (t, J = 6.8 Hz, 3H). |
| 17 | 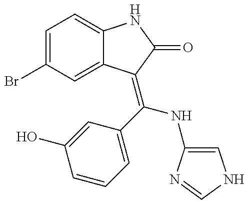 [SMILES: O=C1Nc2ccc(Br)cc2/C1=C(/Nc1c[nH]cn1)c1cccc(O)c1] | (Z)-3-(((1H-imidazol- 4-yl)amino)(3- hydroxyphenyl) methylene)-5- bromoindolin-2-one | 397.05 | ¹H NMR (400 MHz, DMSO-d₆) 8 11.99 (s, 1H), 11.76 (s, 1H), 10.73 (s, 1H), 9.99 (s, 1H), 7.47 (t, J = 7.9 Hz, 1H), 7.43 (d, J = 1.3 Hz, 1H), 7.06 (dd, J = 8.3, 2.5 Hz, 1H), 7.01 (dd, J = 8.3, 2.0 Hz, 1H), 6.88 (dt, J = 7.6, 1.3 Hz, 1H), 6.81-6.76 (m, 2H), 5.85 (d, J = 2.0 Hz, 1H), 5.41- 5.37 (m, 1H). |
| 18 | 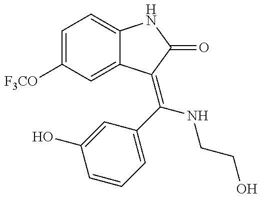 [SMILES: O=C1Nc2ccc(OC(F)(F)F)cc2/C1=C(/NCCO)c1cccc(O)c1] | (Z)-3-(((2- hydroxyethyl) amino)(3-hydroxy phenyl)methylene)-5- (trifluoromethoxy) indolin-2-one | 381.10 | ¹H NMR (400 MHz, DMSO-d₆) § 10.54 (s, 1H), 10.34 (t, J = 6.1 Hz, 1H), 9.87 (s, 1H), 7.44 (t, J = 7.8 Hz, 1H), 7.05-6.97 (m, 1H), 6.84-6.69 (m, 4H), 5.39 (s, 1H), 4.94 (t, J = 5.0 Hz, 1H), 3.48 (q, J = 5.4 Hz, 2H), 3.18 (q, J = 5.6 Hz, 2H). |
| 19 | 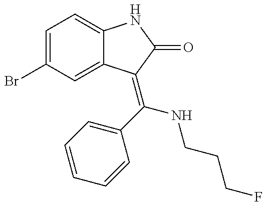 [SMILES: O=C1Nc2ccc(Br)cc2/C1=C(/NCCCF)c1ccccc1] | (Z)-5-bromo-3-(((3- fluoropropyl)amino) (phenyl)methylene) indolin-2-one | 375.05 | ¹H NMR (400 MHz, DMSO-d₆) § 10.56 (s, 1H), 10.31 (t, J = 6.3 Hz, 1H), 7.71-7.63 (m, 3H), 7.49-7.41 (m, 2H), 6.92 (dd, J = 8.2, 2.0 Hz, 1H), 6.72 (d, J = 8.2 Hz, 1H), 5.45 (d, J = 2.0 Hz, 1H), 4.52 (t, J = 5.7 Hz, 1H), 4.40 (t, J = 5.7 Hz, 1H), 3.25- 3.16 (m, 2H), 1.95-1.79 (m, 2H). |
| 20 | 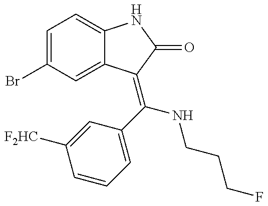 [SMILES: O=C1Nc2ccc(Br)cc2/C1=C(/NCCCF)c1cccc(C(F)F)c1] | (Z)-5-bromo-3-((3- (difluoromethyl) phenyl)((3- fluoropropyl) amino)methylene) indolin-2-one | 427.00 | ¹H NMR (400 MHz, DMSO-d₆) δ 10.62 (s, 1H), 10.32 (t, J = 6.3 Hz, 1H), 7.93-7.80 (m, 2H), 7.74-7.63 (m, 2H), 7.15 (t, J = 55.5 Hz, 1H), 6.94 (dd, J = 8.2, 2.0 Hz, 1H), 6.73 (d, J = 8.2 Hz, 1H), 5.40 (d, J = 2.0 Hz, 1H), 4.52 (t, J = 5.7 Hz, 1H), 4.41 (t, J = 5.7 Hz, 1H), 3.20 (q, J = 6.7 Hz, 2H), 1.96-1.80 (m, 2H). |
| 21 | 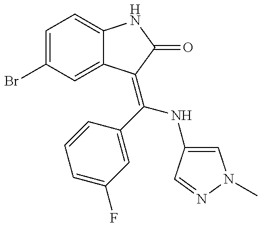 [SMILES: Cn1cc(N/C(=C2\C(=O)Nc3ccc(Br)cc32)c2cccc(F)c2)cn1] | (Z)-5-bromo-3-((3- fluorophenyl)((1- methyl-1H-pyrazol-4- yl)amino)methylene) indolin-2-one | 413.00 | ¹H NMR (400 MHz, DMSO-d₆) 8 11.63 (s, 1H), 10.81 (s, 1H), 7.71-7.63 (m, 1H), 7.54-7.45 (m, 1H), 7.42 (m, 1H), 7.37-7.28 (m, 2H), 7.03 (dd, J = 8.4, 2.0 Hz, 1H), 6.91 (d, J = 0.8 Hz, 1H), 6.79 (d, J = 8.4 Hz, 1H), 5.65 (d, J = 2.0 Hz, 1H), 3.67 (s, 3H). |
| 22 | 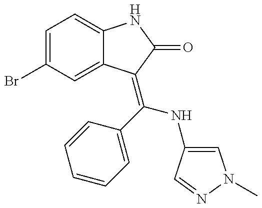 [SMILES: Cn1cc(N/C(=C2\C(=O)Nc3ccc(Br)cc32)c2ccccc2)cn1] | (Z)-5-bromo-3-(((1- methyl-1H-pyrazol-4- yl)amino)(phenyl) methylene)indolin- 2-one | 395.00 | ¹H NMR (400 MHz, DMSO-d₆) 8 11.71 (s, 1H), 10.78 (s, 1H), 7.70-7.57 (m, 3H), 7.45 (d, J = 6.8 Hz, 2H), 7.28 (s, 1H), 7.00 (d, J = 8.3 Hz, 1H), 6.83-6.74 (m, 2H), 5.64 (s, 1H), 3.64 (s, 3H). |

The following intermediate compounds were prepared using General Procedure A or B

| | | MS: | |
|---|---|---|---|
| | | [(M + | |
| Structure | Name | 1)]⁺ | ¹H NMR |
| 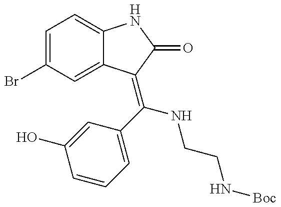 [SMILES: CC(C)(C)OC(=O)NCCN/C(=C1\C(=O)Nc2ccc(Br)cc21)c1cccc(O)c1] | Tert-butyl (Z)-(2-(((5- bromo-2-oxoindolin- 3-ylidene)(3-hydroxy phenyl)methyl)amino) ethyl)carbamate | 474.10 | — |
| 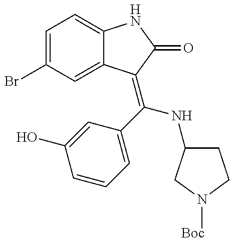 [SMILES: CC(C)(C)OC(=O)N1CCC(N/C(=C2\C(=O)Nc3ccc(Br)cc32)c2cccc(O)c2)C1] | Tert-butyl (Z)-3-(((5- bromo-2-oxoindolin- 3-ylidene)(3-hydroxy phenyl)methyl)amino) pyrrolidine-1- carboxylate | 500.10 | — |
| 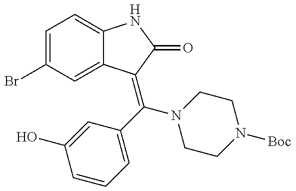 [SMILES: CC(C)(C)OC(=O)N1CCN(/C(=C2\C(=O)Nc3ccc(Br)cc32)c2cccc(O)c2)CC1] | Tert-butyl (Z)-4-((5- bromo-2-oxoindolin- 3-ylidene)(3-hydroxy phenyl)methyl) piperazine- 1-carboxylate | 500.10 | — |
| 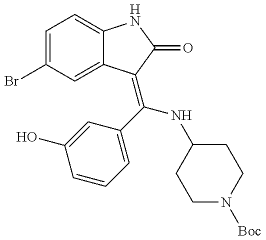 [SMILES: CC(C)(C)OC(=O)N1CCC(N/C(=C2\C(=O)Nc3ccc(Br)cc32)c2cccc(O)c2)CC1] | Tert-butyl (Z)-4-(((5- bromo-2-oxoindolin-3- ylidene)(3-hydroxy phenyl)methyl)amino) piperidine-1- carboxylate | 514.05 | — |
| 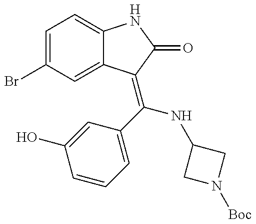 [SMILES: CC(C)(C)OC(=O)N1CC(N/C(=C2\C(=O)Nc3ccc(Br)cc32)c2cccc(O)c2)C1] | Tert-butyl (Z)-3-(((5- bromo-2-oxoindolin-3- ylidene)(3-hydroxy phenyl)methyl)amino) azetidine-1-carboxylate | 486.00 | ¹H NMR (400 MHz, DMSO-d₆) δ 10.48 (d, J = 8.0 Hz, 1H), 7.47 (t, J = 7.9 Hz, 1H), 7.04 (dd, J = 8.1, 2.4 Hz, 1H), 6.98 (dd, J = 8.2, 2.0 Hz, 1H), 6.79 (d, J = 7.5 Hz, 1H), 6.76-6.70 (m, 2H), 5.74 (d, J = 2.0 Hz, 1H), 4.11-4.04 (m, 1H), 3.99-3.92 (m, 2H), 3.87-3.80 (m, 2H), 1.38 (s, 9H). |
| 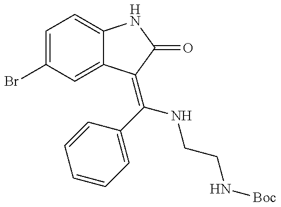 [SMILES: CC(C)(C)OC(=O)NCCN/C(=C1\C(=O)Nc2ccc(Br)cc21)c1ccccc1] | Tert-butyl (Z)-(2-(((5- bromo-2-oxoindolin-3- ylidene)(phenyl)methyl) amino)ethyl)carbamate | 458.10 | ¹H NMR (400 MHz, CDCl₃) δ 10.20 (s, 1H), 8.12 (s, 1H), 7.64- 7.55 (m, 3H), 7.33 (s, 2H), 7.00 (d, J = 7.9 Hz, 1H), 6.74 (s, 1H), 5.64 (s, 1H), 4.92 (s, 1H), 3.28 (s, 4H), 1.42 (s, 9H). |
| 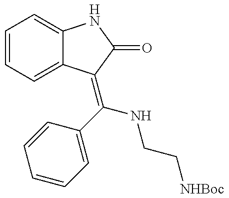 [SMILES: CC(C)(C)OC(=O)NCCN/C(=C1\C(=O)Nc2ccccc21)c1ccccc1] | Tert-butyl (Z)-(2-(((2- oxoindolin-3-ylidene) (phenyl)methyl)amino) ethyl)carbamate | 380.20 | ¹H NMR (400 MHz, CDCl₃) δ 10.17 (s, 1H), 7.93 (s, 1H), 7.63- 7.55 (m, 3H), 7.41-7.35 (m, 2H), 6.97-6.85 (m, 2H), 6.61 (t, J = 7.5 Hz, 1H), 5.68 (d, J = 7.8 Hz, 1H), 4.94 (s, 1H), 3.28 (s, 4H), 1.45 (s, 9H) |
| 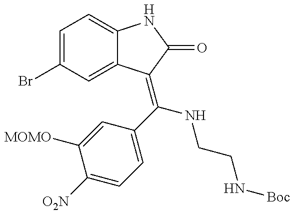 | Tert-butyl (Z)-(2-(((5- bromo-2-oxoindolin-3- ylidene)(3- (methoxymethoxy)-4- nitrophenyl)methyl) amino)ethyl)carbamate | 563.10 | ¹H NMR (400 MHz, CDCl₃) δ 10.20 (s, 1H), 8.30 (s, 1H), 8.11- 7.98 (m, 1H), 7.41 (s, 1H), 7.19- 6.99 (m, 2H), 6.76 (s, 1H), 5.86 (s, 1H), 5.48-5.25 (m, 2H), 4.95 (s, 1H), 4.00 (s, 1H), 3.53 (s, 2H), 3.37-3.15 (m, 4H), 1.40 (s, 9H). |
| 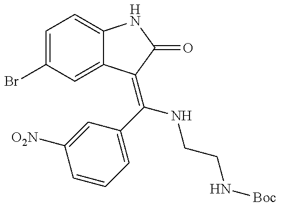 [SMILES: CC(C)(C)OC(=O)NCCN/C(=C1\C(=O)Nc2ccc(Br)cc21)c1cccc([N+](=O)[O-])c1] | Tert-butyl (Z)-(2-(((5- bromo-2-oxoindolin-3- ylidene)(3-nitrophenyl) methyl)amino)ethyl) carbamate | 503.05 | ¹H NMR (400 MHz, DMSO-d₆) δ 10.62 (s, 1H), 10.30 (d, J = 8.1 Hz, 1H), 8.54 (dd, J = 7.9, 2.3, 1H), 8.36 (s, 1H), 8.01-7.92 (m, 2H), 6.97-6.90 (m, 2H), 6.74 (d, J = 8.1 Hz, 1H), 5.38 (d, J = 2.0 Hz, 1H), 3.19-3.07 (m, 2H), 3.06- 2.97 (m, 2H), 1.33 (s, 9H). |
| 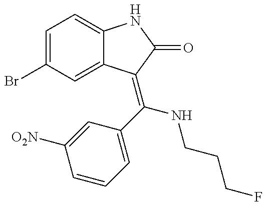 [SMILES: O=C1Nc2ccc(Br)cc2/C1=C(/NCCCF)c1cccc([N+](=O)[O-])c1] | (Z)-5-bromo-3-(((3- fluoropropyl)amino)(3- nitrophenyl)methylene) indolin-2-one | 419.90 | ¹H NMR (400 MHz, DMSO-d₆) δ 10.66 (s, 1H), 10.34 (t, J = 6.3 Hz, 1H), 8.56-8.52 (m, 1H), 8.39- 8.38 (m, 1H), 8.01-7.98 (m, 2H), 6.97 (dd, J = 8.2, 2.0 Hz, 1H), 6.75 (d, J = 8.2 Hz, 1H), 5.43 (d, J = 1.9 Hz, 1H), 4.53 (t, J = 5.7 Hz, 1H), 4.41 (t, J = 5.7 Hz, 1H), 3.23-3.16 (m, 2H), 1.94-1.88 (m, 1H), 1.87-1.81 (m, 1H). |
| 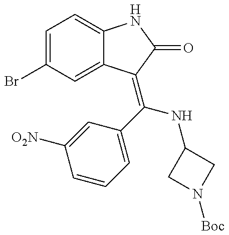 [SMILES: CC(C)(C)OC(=O)N1CC(N/C(=C2\C(=O)Nc3ccc(Br)cc32)c2cccc([N+](=O)[O-])c2)C1] | Tert-butyl (Z)-3-(((5- bromo-2-oxoindolin-3- ylidene)(3-nitrophenyl) methyl)amino)azetidine- 1-carboxylate | 515.35 | ¹H NMR (400 MHz, CDCl₃) δ 10.49 (s, 1H), 8.55-8.50 (m, 1H), 8.21 (t, J = 1.9 Hz, 1H), 8.02 (s, 1H), 7.87 (t, J = 7.9 Hz, 1H), 7.70 (dt, J = 7.5, 1.3 Hz, 1H), 7.07 (dd, J = 8.3, 1.9 Hz, 1H), 6.77 (d, J = 8.3 Hz, 1H), 5.71 (d, J = 1.9 Hz, 1H), 4.13-3.93 (m, 5H), 1.43 (s, 9H). |
| 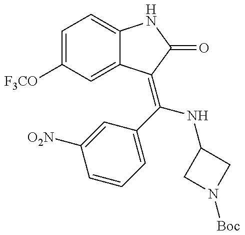 [SMILES: CC(C)(C)OC(=O)N1CC(N/C(=C2\C(=O)Nc3ccc(OC(F)(F)F)cc32)c2cccc([N+](=O)[O-])c2)C1] | Tert-butyl (Z)-3-(((3- nitrophenyl)(2-oxo-5- (trifluoromethoxy) indolin-3-ylidene) methyl)amino) azetidine-1- carboxylate | 521.15 | ¹H NMR (400 MHz, CDCl₃) δ 10.48 (s, 1H), 8.52 (d, J = 8.4 Hz, 1H), 8.23 (s, 1H), 7.86 (t, J = 7.9 Hz, 1H), 7.69 (d, J = 7.6 Hz, 1H), 7.60 (s, 1H), 6.84 (d, J = 8.7 Hz, 1H), 5.44 (s, 1H), 4.12-3.92 (m, 5H), 1.43 (s, 9H). |
| 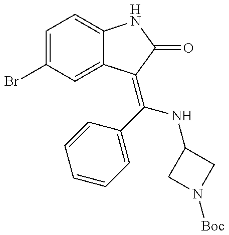 [SMILES: CC(C)(C)OC(=O)N1CC(N/C(=C2\C(=O)Nc3ccc(Br)cc32)c2ccccc2)C1] | Tert-butyl (Z)-3-(((5- bromo-2-oxoindolin-3- ylidene)(phenyl) methyl)amino) azetidine-1- carboxylate | 470.10 | ¹H NMR (400 MHz, DMSO-d₆) δ 10.67 (s, 1H), 10.52 (d, J = 8.2 Hz, 1H), 7.73-7.62 (m, 3H), 7.44- 7.38 (m, 2H), 6.97 (dd, J = 8.2, 2.0 Hz, 1H), 6.74 (d, J = 8.2 Hz, 1H), 5.54 (d, J = 2.0 Hz, 1H), 4.08-3.97 (m, 1H), 3.91 (t, J = 8.1 Hz, 2H), 3.85 (d, J = 5.7 Hz, 2H), 1.36 (s, 9H). |
| 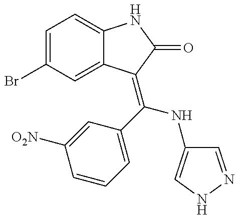 [SMILES: O=C1Nc2ccc(Br)cc2/C1=C(/Nc1cn[nH]c1)c1cccc([N+](=O)[O-])c1] | (Z)-3-(((1H-pyrazol-4- yl)amino)(3-nitro phenyl)methylene)-5- bromoindolin-2-one | 426.00 | ¹H NMR (400 MHz, DMSO-d₆) δ 12.00 (s, 1H), 11.80 (s, 1H), 10.85 (s, 1H), 8.55-8.48 (m, 1H), 8.38 (t, J = 1.8 Hz, 1H), 8.03-7.97 (m, 1H), 7.94 (t, J = 7.9 Hz, 1H), 7.42 (d, J = 1.4 Hz, 1H), 7.05 (dd, J = 8.2, 2.0 Hz, 1H), 6.81 (d, J = 8.3 Hz, 1H), 5.81 (s, 1H), 5.63 (d, J = 2.0 Hz, 1H) |
| 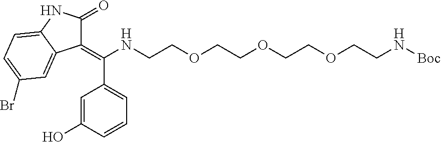 [SMILES: CC(C)(C)OC(=O)NCCOCCOCCOCCN/C(=C1\C(=O)Nc2ccc(Br)cc21)c1cccc(O)c1] | Tert-butyl (Z)-(1-(5- bromo-2-oxoindolin-3- ylidene)-1-(3-hydroxy- phenyl)-5,8,11-trioxa- 2-azatridecan-13- yl)carbamate | 606.20 | ¹H NMR (400 MHz, DMSO-d₆) δ 10.50 (s, 1H), 10.31 (t, J = 6.1 Hz, 1H), 9.89 (s, 1H), 7.45 (t, J = 7.8 Hz, 1H), 7.04-7.00 (m, 1H), 6.93 (dd, J = 8.2, 2.0 Hz, 1H), 6.82 (dt, J = 7.5, 1.2 Hz, 1H), 6.78-6.68 (m, 3H), 5.63 (d, J = 2.0 Hz, 1H), 3.57-3.43 (m, 10H), 3.38-3.33 (m, 2H), 3.28-3.21 (m, 2H), 3.08-2.99 (m, 2H), 1.36 (s, 9H). |
| 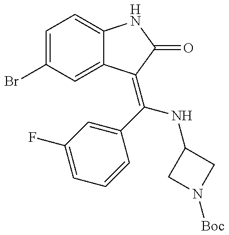 [SMILES: CC(C)(C)OC(=O)N1CC(N/C(=C2\C(=O)Nc3ccc(Br)cc32)c2cccc(F)c2)C1] | (Tert-butyl (Z)-3-(((5- bromo-2-oxoindolin-3- ylidene)(3-fluoro phenyl)methyl)amino) azetidine-1-carboxylate | 487.95 | ¹H NMR (400 MHz, CDCl₃) δ 10.41 (d, J = 6.6 Hz, 1H), 7.89 (s, 1H), 7.66-7.58 (m, 1H), 7.38-7.32 (m, 1H), 7.11 (dt, J = 7.6, 1.2 Hz, 1H), 7.08-6.99 (m, 2H), 6.74 (d, J = 8.2 Hz, 1H), 5.83 (d, J = 1.9 Hz, 1H), 4.10-3.90 (m, 4H), 1.43 (s, 9H). |

### Example 23: (Z)-5-bromo-3-((3-hydroxyphenyl)(piperazin-1-yl)methylene)indolin-2-one

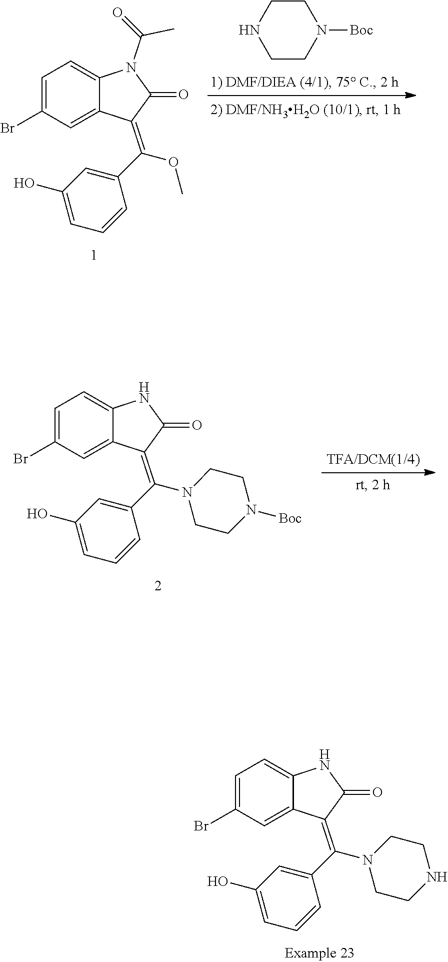 [SMILES: CO/C(=C1\C(=O)N(C(C)=O)c2ccc(Br)cc21)c1cccc(O)c1 ; CC(C)(C)OC(=O)N1CCNCC1 ; CC(C)(C)OC(=O)N1CCN(/C(=C2\C(=O)Nc3ccc(Br)cc32)c2cccc(O)c2)CC1 ; O=C1Nc2ccc(Br)cc2/C1=C(\c1cccc(O)c1)N1CCNCC1]

### Preparation of Tert-butyl (Z)-4-((5-bromo-2-oxoindolin-3-ylidene)(3-hydroxyphenyl) methyl)piperazine-1-carboxylate

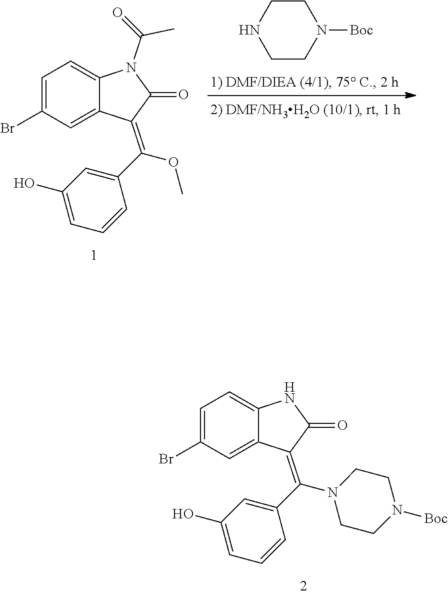 [SMILES: CO/C(=C1\C(=O)N(C(C)=O)c2ccc(Br)cc21)c1cccc(O)c1 ; CC(C)(C)OC(=O)N1CCNCC1 ; CC(C)(C)OC(=O)N1CCN(/C(=C2\C(=O)Nc3ccc(Br)cc32)c2cccc(O)c2)CC1]

General Procedure B was used to prepare compound 2 above. LC/MS (ESI, m/z): [(M+1)]⁺=500.10.

### Preparation of (Z)-5-bromo-3-((3-hydroxyphenyl)(piperazin-1-yl)methylene)indolin-2-one

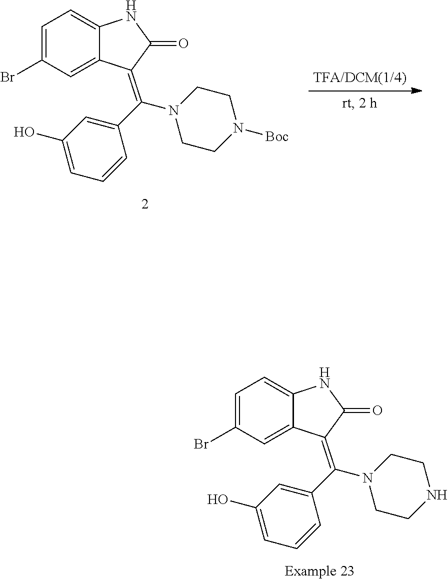 [SMILES: CC(C)(C)OC(=O)N1CCN(/C(=C2\C(=O)Nc3ccc(Br)cc32)c2cccc(O)c2)CC1 ; O=C1Nc2ccc(Br)cc2/C1=C(\c1cccc(O)c1)N1CCNCC1]

A solution of tert-butyl (Z)-4-((5-bromo-2-oxoindolin-3-ylidene)(3-hydroxyphenyl) methyl)piperazine-1-carboxylate (100 mg, 0.20 mmol) in TFA (0.2 mL) and DCM (0.8 mL) was stirred for 2 h at room temperature under nitrogen atmosphere. The resulting mixture was concentrated under reduced pressure. The residue was purified by reverse flash chromatography with the following conditions: Column: Spherical C18, 20-40 μm, 120 g; Eluent A: Water (plus 10 mM NH₄HCO₃), Eluent B: ACN; Flow rate: 50 mL/min; Gradient (B %): 5%-20%, 10 min; 20%-60%, 20 min; 40%-95%; 2 min; 95%, 5 min; Detector: UV 254 nm. The fractions containing desired product were collected at 47% B and concentrated under reduced pressure to afford (Z)-5-bromo-3-((3-hydroxyphenyl)(piperazin-1-yl)methylene)indolin-2-one (75 mg, 94%) as an orange solid: ¹H NMR (400 MHz, DMSO-d₆) δ 10.13 (s, 1H), 9.83 (s, 1H), 7.40 (t, J=7.8 Hz, 1H), 7.03 (dd, J=8.1, 2.4 Hz, 1H), 6.90 (d, J=7.5 Hz, 1H), 6.78-6.88 (m, 2H), 6.62 (d, J=8.2 Hz, 1H), 5.51 (d, J=2.0 Hz, 1H), 3.33 (s, 4H), 2.85 (s, 4H); LC/MS (ESI, m/z): [(M+1)]⁺=400.00.

The following exemplary compounds were prepared using the procedure of Example 23.

| Ex. | | | MS: | |
|---|---|---|---|---|
| No. | Structure | Name | [(M + 1)]⁺ | ¹H NMR |
| 24 | 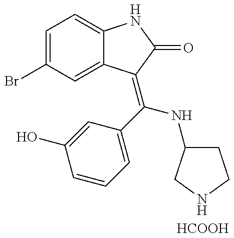 [SMILES: O=C1Nc2ccc(Br)cc2/C1=C(/NC1CCNC1)c1cccc(O)c1 ; "HCOOH"] | (Z)-5-bromo-3-((3- hydroxyphenyl) (pyrrolidin-3- ylamino)methylene) indolin-2-one formate | 400.15 | ¹H NMR (400 MHz, Methanol- d4) δ 8.47 (s, 1H), 7.51-7.31 (m, 1H), 6.79-7.17 (m, 4H), 6.74 (d, J = 8.2 Hz, 1H), 5.76 (d, J = 2.0 Hz, 1H), 3.68-4.22 (m, 3H), 3.44-3.18 (m, 1H), 2.55-2.40 (m, 1H), 2.29-2.06 (m, 1H). |
| 25 | 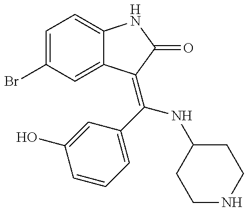 [SMILES: O=C1Nc2ccc(Br)cc2/C1=C(/NC1CCNCC1)c1cccc(O)c1] | (Z)-5-bromo-3-((3- hydroxyphenyl) (piperidin-4- ylamino)methylene) indolin-2-one | 414.10 | ¹H NMR (400 MHz, DMSO-d₆) δ 10.59 (s, 1H), 10.39 (d, J = 9.5 Hz, 1H), 7.47 (t, J = 7.8 Hz, 1H), 7.05 (dd, J = 8.1, 2.5 Hz, 1H), 6.94 (dd, J = 8.2, 2.1 Hz, 1H), 6.84 (d, J = 7.5 Hz, 1H), 6.78 (t, J = 2.0 Hz, 1H), 6.73 (d, J = 8.2 Hz, 1H), 5.61 (d, J = 2.0 Hz, 1H), 3.28-3.16 (m, 2H), 2.92-2.82 (m, 2H), 2.42-2.23 (m, 2H), 1.73 (d, J = 12.1 Hz, 2H), 1.47-1.30 (m, 2H). |
| 26 | 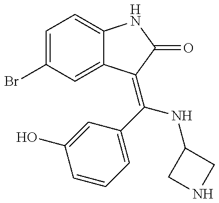 [SMILES: O=C1Nc2ccc(Br)cc2/C1=C(/NC1CNC1)c1cccc(O)c1] | (Z)-3-((azetidin-3- ylamino)(3-hydroxy phenyl)methylene) 5-bromindolin-2- one | 386.05 | ¹H NMR (400 MHz, DMSO-d₆) δ 10.63 (s, 1H), 10.53 (d, J = 8.7 Hz, 1H), 7.46 (t, J = 7.8 Hz, 1H), 7.08-7.01 (m, 1H), 6.96 (dd, J = 8.2, 2.0 Hz, 1H), 6.80- 6.68 (m, 3H), 5.74 (d, J = 2.0 Hz, 1H), 4.11-3.98 (m, 1H), 3.53-3.38 (m, 4H). |
| 27 | 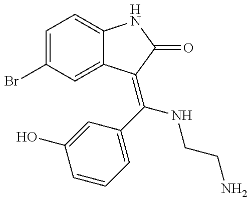 [SMILES: NCCN/C(=C1\C(=O)Nc2ccc(Br)cc21)c1cccc(O)c1] | (Z)-3-(((2-amino ethyl)amino)(3- hydroxyphenyl) methylene)-5-bromo indolin-2-one | 374.00 | ¹H NMR (400 MHz, DMSO-d₆) δ 10.56 (s, 1H), 10.22 (t, J = 6.3 Hz, 1H), 8.27 (s, 1H), 7.47 (t, J = 7.8 Hz, 1H), 7.04 (dd, J = 8.2, 2.4 Hz, 1H), 6.94 (dd, J = 8.2, 2.0 Hz, 1H), 6.86-6.77 (m, 2H), 6.72 (d, J = 8.2 Hz, 1H), 5.60 (d, J = 2.0 Hz, 1H), 3.30-3.21 (m, 2H), 2.80 (t, J = 6.2 Hz, 2H). |
| 28 | 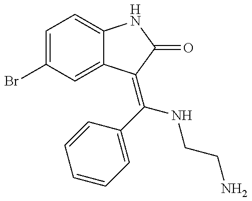 [SMILES: NCCN/C(=C1\C(=O)Nc2ccc(Br)cc21)c1ccccc1] | (Z)-3-(((2- aminoethyl)amino) (phenyl)methylene)- 5-bromoindolin-2- one | 358.00 | ¹H NMR (400 MHz, DMSO-d₆) δ 10.52 (s, 1H), 10.40 (t, J = 6.1 Hz, 1H), 7.69-7.62 (m, 3H), 7.47-7.40 (m, 2H), 6.90 (dd, J = 8.2, 2.0 Hz, 1H), 6.70 (d, J = 8.2 Hz, 1H), 5.43 (d, J = 2.0 Hz, 1H), 3.05 (q, J = 6.1 Hz, 2H), 2.65 (t, J = 6.2 Hz, 2H), 1.59 (s, 2H). |
| 29 | 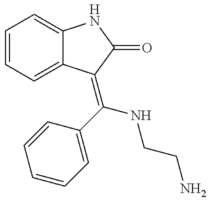 [SMILES: NCCN/C(=C1\C(=O)Nc2ccccc21)c1ccccc1] | (Z)-3-(((2- aminoethyl) amino)(phenyl) methylene)indolin- 2-one | 280.20 | ¹H NMR (400 MHz, DMSO-d₆) δ 10.36 (s, 1H), 10.32 (t, J = 6.2 Hz, 1H), 7.69-7.58 (m, 3H), 7.47-7.38 (m, 2H), 6.82-6.73 (m, 2H), 6.49-6.38 (m, 1H), 5.46 (d, J = 7.8 Hz, 1H), 3.06-2.98 (m, 2H), 2.64 (t, J = 6.2 Hz, 2H), 1.50 (s, 2H) |
| 30 | 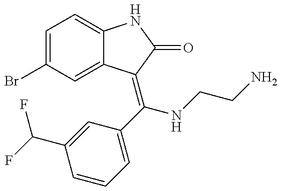 [SMILES: NCCN/C(=C1\C(=O)Nc2ccc(Br)cc21)c1cccc(C(F)F)c1] | (Z)-3-(((2-amino ethyl)amino)(3- (difluoromethyl) phenyl)methylene)- 5-bromoindolin-2- one | 408.05 | ¹H NMR (400 MHz, CDCl₃) δ 10.39 (s, 1H), 7.83-7.73 (m, 2H), 7.67 (s, 1H), 7.60-7.52 (m, 2H), 7.03 (dd, J = 8.3, 1.9 Hz, 1H), 6.85 (t, J = 56.1 Hz, 1H), 6.74 (d, J = 8.3 Hz, 1H), 5.67 (d, J = 1.9 Hz, 1H), 3.23-3.16 (m, 2H), 2.89 (t, J = 6.1 Hz, 2H). |
| 31 | 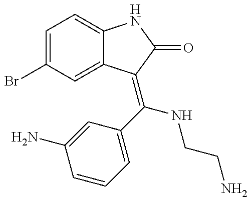 [SMILES: NCCN/C(=C1\C(=O)Nc2ccc(Br)cc21)c1cccc(N)c1] | (Z)-3-(((2-amino ethyl)amino)(3- aminophenyl) methylene)-5-bromo indolin-2-one | 373.05 | ¹H NMR (400 MHz, DMSO-d₆) δ 10.48 (s, 1H), 10.33 (t, J = 6.2 Hz, 1H), 7.27 (t, J = 7.7 Hz, 1H), 6.91 (dd, J = 8.2, 2.1 Hz, 1H), 6.78 (dd, J = 8.2, 2.3 Hz, 1H), 6.69 (d, J = 8.2 Hz, 1H), 6.52 (t, J = 1.9 Hz, 1H), 6.47 (dt, J = 7.4, 1.3 Hz, 1H), 5.77 (d, J = 2.0 Hz, 1H), 5.44 (s, 2H), 3.14- 3.07 (m, 2H), 2.65 (t, J = 6.3 Hz, 2H), 2.08-1.88 (m, 2H). |
| 32 | 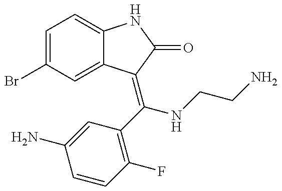 [SMILES: NCCN/C(=C1\C(=O)Nc2ccc(Br)cc21)c1cc(N)ccc1F] | (Z)-3-((5-amino-2- fluorophenyl)((2- aminoethyl)amino) methylene)-5-bromo indolin-2-one | 391.05 | ¹H NMR (400 MHz, DMSO-d₆) δ 10.55 (s, 1H), 10.33 (t, J = 5.9 Hz, 1H), 7.17 (t, J = 9.1 Hz, 1H), 7.00-6.92 (m, 1H), 6.84- 6.78 (m, 1H), 6.72 (d, J = 8.2 Hz, 1H), 6.51 (dd, J = 5.9, 2.8 Hz, 1H), 5.82 (d, J = 2.0 Hz, 1H), 5.33 (s, 2H), 3.15-3.07 (m, 2H), 2.66 (t, J = 6.2 Hz, 2H). |
| 33 | 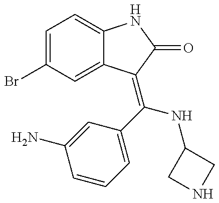 [SMILES: Nc1cccc(/C(NC2CNC2)=C2/C(=O)Nc3ccc(Br)cc32)c1] | (Z)-3-((3-amino phenyl)(azetidin-3- ylamino)methylene)- 5-bromoindolin-2- one | 385.00 | ¹H NMR (400 MHz, DMSO-d₆) δ 10.61 (s, 1H), 10.51 (d, J = 8.9 Hz, 1H), 7.27 (t, J = 7.8 Hz, 1H), 6.95 (dd, J = 8.2, 2.0 Hz, 1H), 6.77-6.83 (m, 1H), 6.72 (d, J = 8.2 Hz, 1H), 6.46 (t, J = 1.9 Hz, 1H), 6.42-6.35 (m, 1H), 5.88 (d, J = 2.0 Hz, 1H), 5.46 (s, 2H), 4.13-4.02 (m, 1H), 3.38- 3.53 (m, 4H). |
| 34 | 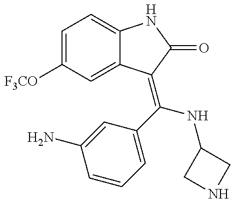 [SMILES: Nc1cccc(/C(NC2CNC2)=C2/C(=O)Nc3ccc(OC(F)(F)F)cc32)c1] | (Z)-3-((3-amino phenyl)(azetidin-3- ylamino)methylene)- 5-(trifluoromethoxy) indolin-2-one | 391.00 | ¹H NMR (400 MHz, DMSO-d₆) δ 10.63 (s, 1H), 10.51 (d, J = 8.8 Hz, 1H), 7.26 (t, J = 7.8 Hz, 1H), 6.85-6.74 (m, 3H), 6.48 (t, J = 1.9 Hz, 1H), 6.41 (d, J = 7.4 Hz, 1H), 5.67 (s, 1H), 5.44 (s, 2H), 4.18-4.06 (m, 1H), 3.52- 3.40 (m, 4H). |
| 35 | 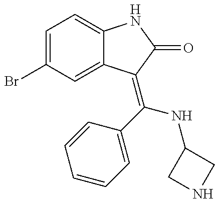 [SMILES: O=C1Nc2ccc(Br)cc2/C1=C(/NC1CNC1)c1ccccc1] | (Z)-3-((azetidin-3- ylamino)(phenyl) methylene)-5-bromo indolin-2-one | 370.00 | ¹H NMR (400 MHz, DMSO-d₆) δ 10.67 (s, 1H), 10.55 (d, J = 8.6 Hz, 1H), 7.72-7.62 (m, 3H), 7.43-7.36 (m, 2H), 6.96 (dd, J = 8.2, 2.0 Hz, 1H), 6.73 (d, J = 8.2 Hz, 1H), 5.53 (d, J = 2.0 Hz, 1H), 4.07-3.96 (m, 1H), 3.96- 3.76 (m, 1H), 3.63-3.43 (m, 4H). |
| 36 | 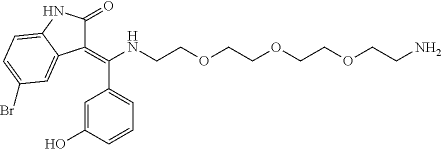 [SMILES: NCCOCCOCCOCCN/C(=C1\C(=O)Nc2ccc(Br)cc21)c1cccc(O)c1] | (Z)-3-(13-amino-1- (3-hydroxyphenyl)- 5,8,11-trioxa-2- azatridecylidene)-5- bromoindolin-2-one | 506.10 | ¹H NMR (400 MHz, CDCl₃) δ 10.27 (s, 1H), 8.26 (s, 1H), 7.42 (t, J = 7.8 Hz, 1H), 7.18-6.89 (m, 3H), 6.81-6.66 (m, 2H), 5.90 (s, 1H), 3.67-3.34 (m, 16H), 2.81-2.87 (m, 2H). |
| 37 | 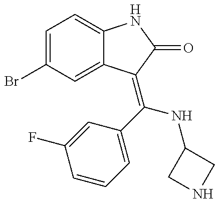 [SMILES: O=C1Nc2ccc(Br)cc2/C1=C(/NC1CNC1)c1cccc(F)c1] | (Z)-3-((azetidin-3- ylamino)(3-fluoro phenyl)methylene)- 5-bromoindolin-2- one | 388.00 | ¹H NMR (400 MHz, DMSO-d₆) δ 10.70 (s, 1H), 10.54 (d, J = 8.6 Hz, 1H), 7.78-7.70 (m, 1H), 7.55 (td, J = 8.8, 2.7 Hz, 1H), 7.39 (dt, J = 9.1, 2.0 Hz, 1H), 7.26 (d, J = 7.5 Hz, 1H), 6.99 (dd, J = 8.2, 2.0 Hz, 1H), 6.75 (d, J = 8.2 Hz, 1H), 5.57 (d, J = 2.0 Hz, 1H), 4.07-3.95 (m, 1H), 3.53-3.37 (m, 4H). |

### Example 38: (Z)-5-bromo-3-((3-hydroxyphenyl)(pyridazin-4-ylamino)methylene) indolin-2-one

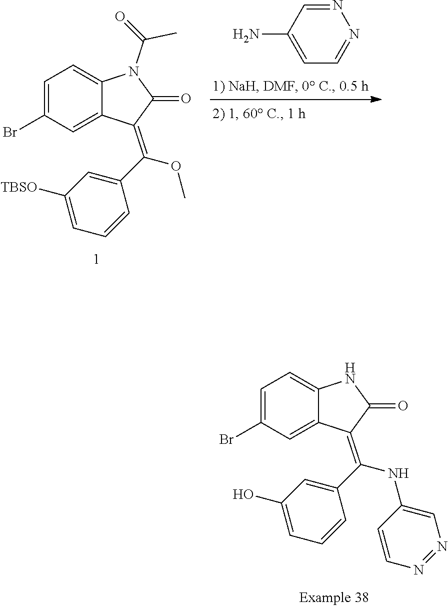 [SMILES: CO/C(=C1\C(=O)N(C(C)=O)c2ccc(Br)cc21)c1cccc(O[Si](C)(C)C(C)(C)C)c1 ; Nc1ccnnc1 ; O=C1Nc2ccc(Br)cc2/C1=C(/Nc1ccnnc1)c1cccc(O)c1]

To a stirred solution of 4-pyridazinamine (190 mg, 1.99 mmol, 5 equiv) in DMF (4 mL) was added NaH (64 mg, 1.59 mmol, 60% in mineral oil) in portions at 0° C. under nitrogen atmosphere. The resulting mixture was stirred for 0.5 h at 0° C. followed by the addition of (Z)-1-acetyl-5-bromo-3-((3-((tert-butyldimethylsilyl)oxy)phenyl)(methoxy)methylene)indolin-2-one (200 mg, 0.40 mmol). After stirring for additional 1 h at 60° C., the resulting mixture was purified by reverse phase flash chromatography with the following conditions: Column: Spherical C18, 20-40 μm, 120 g; Eluent A: Water (plus 10 mM NH₄HCO₃); Eluent B: ACN; Flow rate: 60 mL/min; Gradient (B %): 5%-5%, 5 min, 40%-65%, 20 min; Detector: UV 220 nm. The fractions containing the desired product were collected at 58% B and concentrated under reduced pressure to give crude desired product which was further purified by Prep-HPLC with the following conditions: Column: XSelect CSH Prep C18 OBD Column, 19*250 mm, 5 μm; Eluent A: Water (10 mmol/L NH₄HCO₃), Eluent B: MeOH; Flow rate: 20 mL/min; Gradient: 67% B to 67% B in 11 min; Detector: 254 nm; RT1: 9.1 min. Desired fractions were collected and concentrated under reduced pressure to afford (Z)-5-bromo-3-((3-hydroxyphenyl)(pyridazin-4-ylamino) methylene)indolin-2-one (71 mg, 44%) as a yellow solid: ¹H NMR (400 MHz, DMSO-d₆) δ 11.87 (s, 1H), 11.04 (s, 1H), 10.04 (s, 1H), 8.82-8.76 (m, 1H), 8.74-8.69 (m, 1H), 7.51 (t, J=7.9 Hz, 1H), 7.16 (dd, J=8.3, 2.0 Hz, 1H), 7.14-7.07 (m, 1H), 7.03-6.97 (m, 1H), 6.93-6.87 (m, 1H), 6.83 (d, J=8.2 Hz, 1H), 6.61 (dd, J=6.0, 3.0 Hz, 1H), 6.06 (d, J=2.0 Hz, 1H); LC/MS (ESI, m/z): [(M+1)]⁺=409.85.

### Example 39 was Prepared Using the Procedure of Example 38

| Ex. | | | MS: | |
|---|---|---|---|---|
| No. | Structure | Name | [(M + 1)]⁺ | ¹H NMR |
| 39 | 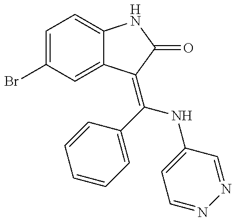 [SMILES: O=C1Nc2ccc(Br)cc2/C1=C(/Nc1ccnnc1)c1ccccc1] | (Z)-5-bromo-3- (phenyl(pyridazin-4- ylamino)methylene) indolin-2-one | 393.05 | ¹HNMR (400 MHz, DMSO-d₆) δ 11.90 (s, 1H), 11.07 (s, 1H), 8.86- 8.61 (m, 2H), 7.80-7.66 (m, 3H), 7.61 (dt, J = 6.8, 1.5 Hz, 2H), 7.16 (dd, J = 8.2, 2.0 Hz, 1H), 6.83 (d, J = 8.3 Hz, 1H), 6.57 (dd, J = 6.1, 3.0 Hz, 1H), 5.88 (d, J = 2.0 Hz, 1H). |

### Example 40: (Z)-5-bromo-3-((3-fluorophenyl)(pyridazin-4-ylamino)methylene) indolin-2-one

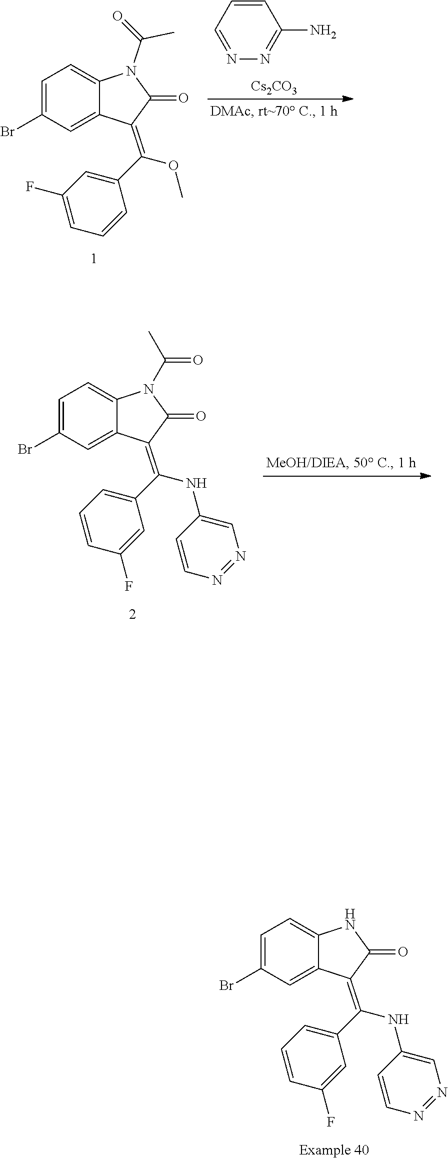

### Preparation of (Z)-1-acetyl-5-bromo-3-((3-fluorophenyl)(pyridazin-4-ylamino) methylene)indolin-2-one

A mixture of 4-pyridazinamine (122 mg, 1.28 mmol) and Cs₂CO₃ (125 mg, 0.38 mmol) in DMAc (2 mL) was stirred for 30 min at room temperature under nitrogen atmosphere followed by the addition of (Z)-1-acetyl-5-bromo-3-((3-fluorophenyl)(methoxy) methylene)indolin-2-one (100 mg, 0.26 mmol) at room temperature. The resulting mixture was stirred for 1 h at 70° C. The mixture was allowed to cool down to room temperature. The resulting mixture was purified by reverse phase flash with the following conditions Column: Spherical C18, 20-40 μm, 120 g; Eluent A: Water (plus 10 mM NH₄HCO₃), Eluent B: ACN; Flow rate: 50 mL/min; Gradient (B %): 5%-22%, 4 min; 65%-85%, 20 min; 40%-95%; 2 min; 95%, 5 min; Detector: UV 254 nm. The fractions containing desired product were collected at 78% B and concentrated under reduced pressure to afford (Z)-1-acetyl-5-bromo-3-((3-fluorophenyl)(pyridazin-4-ylamino)methylene)indolin-2-one (85 mg, 73.17%) as a yellow solid. ¹H NMR (400 MHz, CDCl₃) δ 12.02 (s, 1H), 8.82-8.76 (m, 1H), 8.74 (dd, J=3.0, 1.0 Hz, 1H), 8.20 (d, J=8.8 Hz, 1H), 7.77-7.70 (m, 1H), 7.54-7.44 (m, 1H), 7.36-7.26 (m, 2H), 7.21 (dt, J=8.6, 1.9 Hz, 1H), 6.39 (dd, J=6.0, 3.0 Hz, 1H), 6.10 (d, J=2.1 Hz, 1H), 2.83 (s, 3H); LC/MS (ESI, m/z): [(M+1)]⁺=453.00.

### Preparation of (Z)-5-bromo-3-((3-fluorophenyl)(pyridazin-4-ylamino)methylene)indolin-2-one

A mixture of (Z)-1-acetyl-5-bromo-3-((3-fluorophenyl)(pyridazin-4-ylamino)methylene) indolin-2-one (80 mg, 0.18 mmol) in MeOH (1.5 mL) and DIEA (0.3 mL) was stirred for 1 h at 50° C. The resulting mixture was purified by reverse phase flash with the following conditions Column: Spherical C18, 20-40 μm, 120 g; Eluent A: Water (plus 10 mM NH₄HCO₃), Eluent B: ACN; Flow rate: 50 mL/min; Gradient (B %): 5%-22%, 4 min; 22%-40%, 20 min; 40%-95%; 2 min; 95%, 5 min; Detector: UV 254 nm. The fractions containing desired product were collected at 56% B and concentrated under reduced pressure to afford (Z)-5-bromo-3-((3-fluorophenyl) (pyridazin-4-ylamino)methylene)indolin-2-one (11 mg, 15%) as a yellow solid. ¹H NMR (400 MHz, DMSO-d₆) δ 11.86 (s, 1H), 11.08 (s, 1H), 8.83-8.72 (m, 2H), 7.75 (s, 1H), 7.69-7.52 (m, 2H), 7.47 (d, J=7.5 Hz, 1H), 7.18 (dd, J=8.3, 2.0 Hz, 1H), 6.84 (d, J=8.3 Hz, 1H), 6.61 (dd, J=6.0, 3.0 Hz, 1H), 5.91 (s, 1H); LC/MS (ESI, m/z): [(M+1)]⁺=410.95.

The following exemplary compounds were prepared using the procedure of Example 40.

| Ex. | | | MS: | |
|---|---|---|---|---|
| No. | Structure | Name | [(M + 1)]⁺ | ¹H NMR |
| 41 | 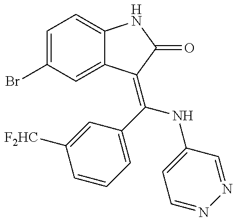 [SMILES: O=C1Nc2ccc(Br)cc2/C1=C(/Nc1ccnnc1)c1cccc(C(F)F)c1] | (Z)-5-bromo-3-((3- (difluoromethyl) phenyl)(pyridazin-4- ylamino)methylene) indolin-2-one | 443.05 | ¹H NMR (400 MHz, DMSO-d₆) δ 11.86 (s, 1H), 11.09 (s, 1H), 8.83- 8.69 (m, 2H), 8.02-7.71 (m, 4H), 7.33-6.95 (m, 2H), 6.83 (d, J = 8.2 Hz, 1H), 6.59 (dd, J = 6.0, 3.0 Hz, 1H), 5.85 (s, 1H). |
| 42 | 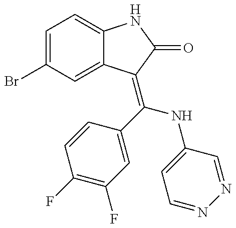 [SMILES: O=C1Nc2ccc(Br)cc2/C1=C(/Nc1ccnnc1)c1ccc(F)c(F)c1] | (Z)-5-bromo-3-((3,4- difluorophenyl) (pyrazin-4- ylamino)methylene) indolin-2-one | 429.00 | ¹H NMR (400 MHz, DMSO-d₆) δ 11.81 (s, 1H), 11.10 (s, 1H), 8.88- 8.74 (m, 2H), 7.93-7.76 (m, 2H), 7.53 (s, 1H), 7.20 (dd, J = 8.3, 2.0 Hz, 1H), 6.85 (d, J = 8.3 Hz, 1H), 6.59 (dd, J = 6.1, 3.0 Hz, 1H), 5.96 (s, 1H). |
| 43 | 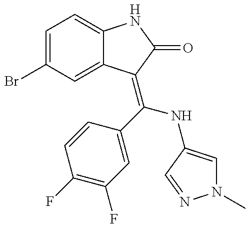 [SMILES: Cn1cc(N/C(=C2\C(=O)Nc3ccc(Br)cc32)c2ccc(F)c(F)c2)cn1] | (Z)-5-bromo-3-((3,4- difluorophenyl)((1- methyl-1H-pyrazol-4- yl)amino)methylene) indolin-2-one | 431.00 | ¹H NMR (400 MHz, DMSO-d₆) δ 11.63 (s, 1H), 10.84 (s, 1H), 7.77- 7.68 (m, 2H), 7.39-7.34 (m, 1H), 7.32 (s, 1H), 7.05 (dd, J = 8.2, 2.0 Hz, 1H), 6.99 (d, J = 0.8 Hz, 1H), 6.80 (d, J = 8.2 Hz, 1H), 5.70 (d, J = 2.0 Hz, 1H), 3.68 (s, 3H). |
| 44 | 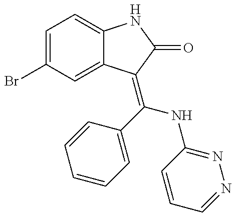 [SMILES: O=C1Nc2ccc(Br)cc2/C1=C(/Nc1cccnn1)c1ccccc1] | (Z)-5-bromo-3- (phenyl(pyridazin-3- ylamino)methylene) indolin-2-one | 393.05 | ¹H NMR (400 MHz, DMSO-d₆) δ 12.52 (s, 1H), 11.08 (s, 1H), 8.83 (d, J = 4.8 Hz, 1H), 7.75-7.63 (m, 3H), 7.58 (d, J = 7.2 Hz, 2H), 7.38 (dd, J = 9.2, 4.8 Hz, 1H), 7.13 (dd, J = 8.0, 2.0 Hz, 1H), 6.85 (d, J = 8.4 Hz, 1H), 6.47 (d, J = 9.2 Hz, 1H), 5.80 (d, J = 2.0 Hz, 1H) |
| 45 | 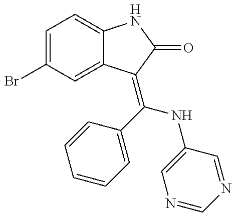 [SMILES: O=C1Nc2ccc(Br)cc2/C1=C(/Nc1cncnc1)c1ccccc1] | (Z)-5-bromo-3- (phenyl(pyrimidin-5- ylamino)methylene) indolin-2-one | 393.00 | ¹H NMR (400 MHz, DMSO-d₆) δ 11.78 (s, 1H), 10.99 (s, 1H), 8.79 (s, 1H), 8.35 (s, 2H), 7.64-7.57 (m, 3H), 7.55-7.50 (m, 2H), 7.10 (dd, J = 8.3, 2.0 Hz, 1H), 6.83 (d, J = 8.2 Hz, 1H), 5.81 (d, J = 2.0 Hz, 1H). |
| 46 | 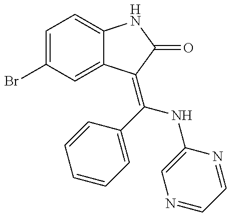 [SMILES: O=C1Nc2ccc(Br)cc2/C1=C(/Nc1cnccn1)c1ccccc1] | (Z)-5-bromo-3- (phenyl(pyrazin-2- ylamino)methylene) indolin-2-one | 393.05 | ¹H NMR (400 MHz, DMSO-d₆) δ 12.27 (s, 1H), 11.02 (s, 1H), 8.26 (dd, J = 2.6, 1.5 Hz, 1H), 8.19 (d, J = 2.6 Hz, 1H), 7.76-7.63 (m, 3H), 7.61-7.54 (m, 3H), 7.13 (dd, J = 8.3, 2.0 Hz, 1H), 6.83 (d, J = 8.2 Hz, 1H), 5.82 (d, J = 2.0 Hz, 1H). |
| 47 | 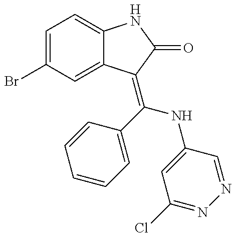 [SMILES: O=C1Nc2ccc(Br)cc2/C1=C(/Nc1cnnc(Cl)c1)c1ccccc1] | (Z)-5-bromo-3-((6- chloropyridazin-4- ylamino)(phenyl) methylene)indolin-2- one | 427.00 | ¹H NMR (400 MHz, DMSO-d₆) δ 11.87 (s, 1H), 11.10 (s, 1H), 8.73 (s, 1H), 7.84-7.67 (m, 3H), 7.63 (d, J = 7.5 Hz, 2H), 7.19 (d, J = 8.3 Hz, 1H), 6.84 (d, J = 8.3 Hz, 1H), 6.57 (s, 1H), 5.96 (s, 1H). |
| 48 | 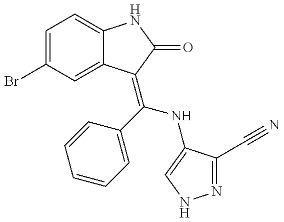 [SMILES: N#Cc1n[nH]cc1N/C(=C1\C(=O)Nc2ccc(Br)cc21)c1ccccc1] | (Z)-4-(((5-bromo-2- oxoindolin-3- ylidene)(phenyl) methyl)amino)-1H- pyrazole-3- carbonitrile | 405.85 | ¹H NMR (400 MHz, DMSO-d₆) δ 13.70 (s, 1H), 11.71 (s, 1H), 10.90 (s, 1H), 7.66-7.54 (m, 3H), 7.50- 7.42 (m, 2H), 7.10-7.00 (m, 2H), 6.81 (d, J = 8.2 Hz, 1H), 5.73 (d, J = 2.0 Hz, 1H). |

### Example 49: (Z)-5-bromo-3-(phenyl(thiazol-5-ylamino)methylene)indolin-2-one

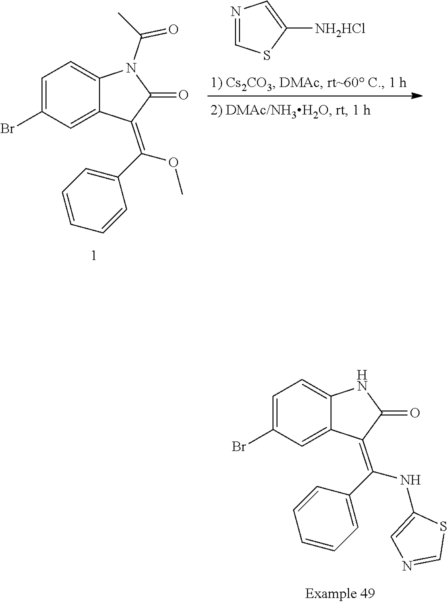

A mixture of thiazol-5-amine hydrochloride (74 mg, 0.54 mmol) and Cs₂CO₃ (70 mg, 0.21 mmol) in DMAc (1 mL) was stirred for 1 h at room temperature followed by the addition of (Z)-1-acetyl-5-bromo-3-(methoxy(phenyl)methylene)indolin-2-one (40 mg, 0.11 mmol) at room temperature. After stirring for additional 1 h at 60° C., the mixture was allowed to cool down to room temperature. To the above mixture was added NH₃·H₂O (0.1 mL) dropwise at room temperature. The resulting mixture was stirred for additional 1 h at room temperature. The resulting mixture was purified by reverse phase flash chromatography with the following conditions: Column: Spherical C18, 20-40 μm, 120 g; Eluent A: water (plus 10 mM NH₄HCO₃); Eluent: ACN; Flow rate: 50 mL/min; Gradient: 70% B-95% B in 20 min; Detector: UV 254 nm. The fractions containing desired product were collected at 77% B and concentrated under reduced pressure to afford (Z)-5-bromo-3-(phenyl(thiazol-5-ylamino)methylene)indolin-2-one (13 mg, 31%) as a yellow solid: ¹H NMR (400 MHz, DMSO-d₆) δ 12.23 (s, 1H), 10.96 (s, 1H), 8.56 (s, 1H), 7.72-7.59 (m, 3H), 7.57 (s, 1H), 7.52 (d, J=7.4 Hz, 2H), 7.08 (d, J=8.1 Hz, 1H), 6.81 (d, J=8.1 Hz, 1H), 5.72 (d, J=2.3 Hz, 1H); LC/MS (ESI, m/z): [(M+1)]⁺=397.80.

The following exemplary compounds were prepared using the procedure of Example 49.

| Ex. No. | Structure | Name | MS: [(M + 1)]⁺ | ¹H NMR |
|---|---|---|---|---|
| 50 | 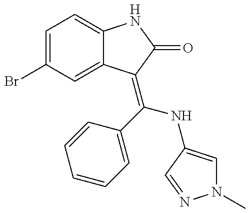 [SMILES: Cn1cc(N/C(=C2\C(=O)Nc3ccc(Br)cc32)c2ccccc2)cn1] | (Z)-5-bromo-3-(((1- methyl-1H-pyrazol-4- yl)amino)(phenyl) methylene)indolin-2-one | 395.00 | ¹H NMR (400 MHz, DMSO-d₆) δ 11.71 (s, 1H), 10.78 (s, 1H), 7.70-7.57 (m, 3H), 7.45 (d, J = 6.8 Hz, 2H), 7.28 (s, 1H), 7.00 (d, J = 8.3 Hz, 1H), 6.83-6.74 (m, 2H), 5.64 (s, 1H), 3.64 (s, 3H). |
| 51 | 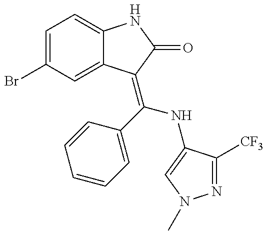 [SMILES: Cn1cc(N/C(=C2\C(=O)Nc3ccc(Br)cc32)c2ccccc2)c(C(F)(F)F)n1] | (Z)-5-bromo-3-(((1-methyl-3- (trifluoromethyl)-1H- pyrazol-4- yl)amino)(phenyl) methylene)indolin-2-one | 463.10 | ¹H NMR (400 MHz, DMSO-d₆) δ 11.70 (s, 1H), 10.87 (s, 1H), 7.70-7.58 (m, 3H), 7.46-7.39 (m, 2H), 7.07 (dd, J = 10.1, 1.7 Hz, 2H), 6.81 (d, J = 8.2 Hz, 1H), 5.68 (d, J = 1.9 Hz, 1H), 3.70 (s, 3H). |
| 52 | 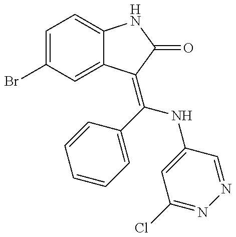 [SMILES: O=C1Nc2ccc(Br)cc2/C1=C(/Nc1cnnc(Cl)c1)c1ccccc1] | (Z)-5-bromo-3-((6- chloropyridazin-4- ylamino)(phenyl) methylene)indolin-2-one | 427.00 | ¹H NMR (400 MHz, DMSO-d₆) δ 11.87 (s, 1H), 11.10 (s, 1H), 8.73 (s, 1H), 7.84-7.67 (m, 3H), 7.63 (d, J = 7.5 Hz, 2H), 7.19 (d, J = 8.3 Hz, 1H), 6.84 (d, J = 8.3 Hz, 1H), 6.57 (s, 1H), 5.96 (s, 1H). |
| 53 | 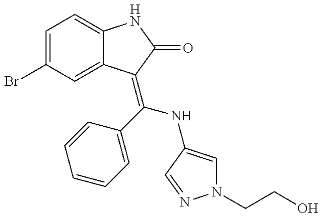 [SMILES: O=C1Nc2ccc(Br)cc2/C1=C(/Nc1cnn(CCO)c1)c1ccccc1] | (Z)-5-bromo-3-(((1- (2-hydroxyethyl)-1H- pyrazol-4- yl)amino)(phenyl) methylene)indolin-2-one | 425.10 | ¹H NMR (400 MHz, DMSO-d₆) δ 11.77 (s, 1H), 10.78 (s, 1H), 7.70-7.56 (m, 3H), 7.46 (d, J = 6.9 Hz, 2H), 7.29 (s, 1H), 7.00 (d, J = 8.4 Hz, 1H), 6.78 (d, J = 6.9 Hz, 2H), 5.66 (s, 1H), 4.82- 4.77 (m, 1H), 3.96-3.88 (m, 2H), 3.64-3.50 (m, 2H). |
| 54 | 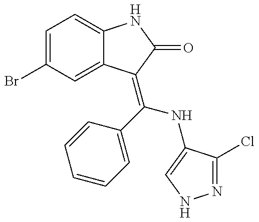 [SMILES: O=C1Nc2ccc(Br)cc2/C1=C(/Nc1c[nH]nc1Cl)c1ccccc1] | (Z)-5-bromo-3-((3- chloro-1H-pyrazol-4- ylamino)(phenyl) methylene)indolin-2-one | 415.00 | ¹H NMR (400 MHz, DMSO-d₆) δ 11.55 (s, 1H), 10.84 (s, 1H), 7.68-7.57 (m, 3H), 7.49-7.41 (m, 2H), 7.05 (dd, J = 8.2, 2.0 Hz, 1H), 6.87-6.77 (m, 2H), 5.70 (t, J = 1.5 Hz, 1H). |
| 55 | 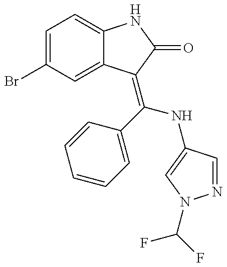 [SMILES: O=C1Nc2ccc(Br)cc2/C1=C(/Nc1cnn(C(F)F)c1)c1ccccc1] | (Z)-5-bromo-3-(((1- (difluoromethyl)-1H- pyrazol-4- yl)amino)(phenyl) methylene)indolin-2-one | 431.00 | ¹H NMR (400 MHz, DMSO-d₆) δ 11.66 (s, 1H), 10.86 (s, 1H), 7.80-7.59 (m, 5H), 7.49 (dd, J = 7.7, 2.0 Hz, 2H), 7.26 (s, 1H), 7.04 (dt, J = 8.3, 1.5 Hz, 1H), 6.79 (d, J = 8.2 Hz, 1H), 5.69 (d, J = 1.8 Hz, 1H). |

### Example 56: (Z)-3-(((1H-imidazol-4-yl)amino)(3-hydroxyphenyl)methylene)-5-bromoindolin-2-one

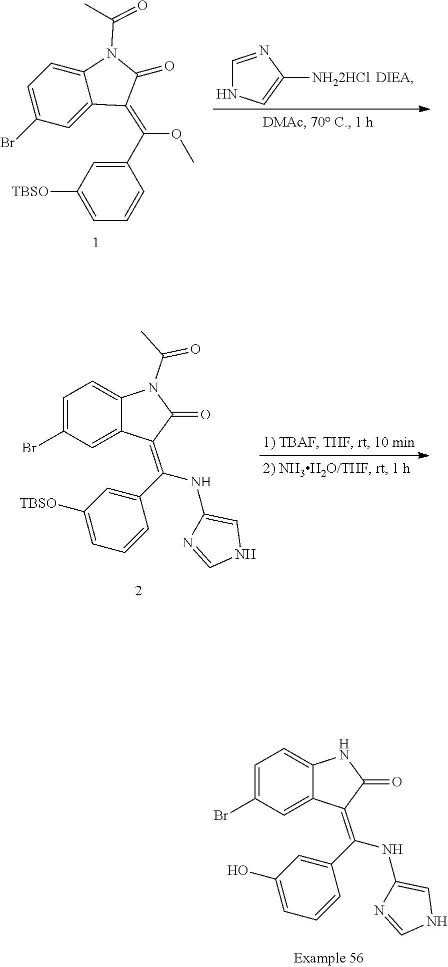

### Preparation of (Z)-3-(((1H-imidazol-4-yl)amino)(3-((tert-butyldimethylsilyl)oxy) phenyl)methylene)-1-acetyl-5-bromoindolin-2-one

A mixture of (Z)-1-acetyl-5-bromo-3-((3-((tert-butyldimethylsilyl)oxy)phenyl)(methoxy) methylene) indolin-2-one (250 mg, 0.50 mmol), 4-aminoimidazole (207 mg, 2.49 mmol) and DIEA (385.83 mg, 2.99 mmol) in DMAc (5 mL) was stirred for 1 h at 70° C. under nitrogen atmosphere. The mixture was purified by reverse flash chromatography with the following conditions: Column: Spherical C18, 20-40 μm, 330 g; Eluent A: Water (plus 10 mM NH₄HCO₃), Eluent B: ACN; Flow rate: 80 mL/min; Gradient (B %): 5%-25%, 6 min; 25%-50%, 4 min; 50%-65%, 3 min; 65%-85%, 5 min; 85%-95%, 15 min; Detector: UV 220 nm. The fractions containing desired product were collected at 95% B and concentrated under reduced pressure to afford (Z)-3-(((1H-imidazol-4-yl)amino)(3-((tert-butyldimethylsilyl)oxy)phenyl)methylene)-1-acetyl-5-bromoindolin-2-one (175 mg, 63.54%) as a yellow solid: ¹H NMR (400 MHz, DMSO-d₆) δ 11.99 (s, 1H), 11.89 (s, 1H), 8.05 (d, J=8.6 Hz, 1H), 7.61 (t, J=7.9 Hz, 1H), 7.49 (d, J=1.3 Hz, 1H), 7.23-7.19 (m, 1H), 7.17-7.10 (m, 2H), 6.99-6.96 (m, 1H), 5.78 (d, J=2.1 Hz, 1H), 5.44 (s, 1H), 2.72 (s, 3H), 0.94 (s, 9H), 0.17 (d, J=7.5 Hz, 6H); LC/MS (ESI, m/z): [(M+1)]⁺=553.15.

### Preparation of (Z)-3-(((1H-imidazol-4-yl)amino)(3-hydroxyphenyl)methylene)-5-bromoindolin-2-one (Example 56)

A mixture of (Z)-3-(((1H-imidazol-4-yl)amino)(3-((tert-butyldimethylsilyl)oxy)phenyl) methylene)-1-acetyl-5-bromoindolin-2-one (150 mg, 0.27 mmol) and TBAF (106 mg, 0.41 mmol) in THF (3 mL) was stirred for 10 min at room temperature under nitrogen atmosphere. To the above mixture was added NH₃·H₂O (1 mL) at room temperature under nitrogen atmosphere. The resulting mixture was stirred for 1 h at room temperature under nitrogen atmosphere. The resulting mixture was concentrated under reduced pressure. The residue was purified by Prep-TLC (CH₂Cl₂/MeOH 15:1) to afford (Z)-3-(((1H-imidazol-4-yl)amino)(3-hydroxyphenyl) methylene)-5-bromoindolin-2-one (80 mg, 75%) as a yellow solid: ¹H NMR (400 MHz, DMSO-d₆) δ 11.99 (s, 1H), 11.76 (s, 1H), 10.73 (s, 1H), 9.99 (s, 1H), 7.47 (t, J=7.9 Hz, 1H), 7.43 (d, J=1.3 Hz, 1H), 7.06 (dd, J=8.3, 2.5 Hz, 1H), 7.01 (dd, J=8.3, 2.0 Hz, 1H), 6.88 (dt, J=7.6, 1.3 Hz, 1H), 6.81-6.76 (m, 2H), 5.85 (d, J=2.0 Hz, 1H), 5.41-5.37 (m, 1H); LC/MS (ESI, m/z): [(M+1)]⁺=397.05.

### Example 57: (Z)-6-(((2-aminoethyl)amino)(5-bromo-2-oxoindolin-3-ylidene)methyl) benzo[d]oxazol-2(3H)-one

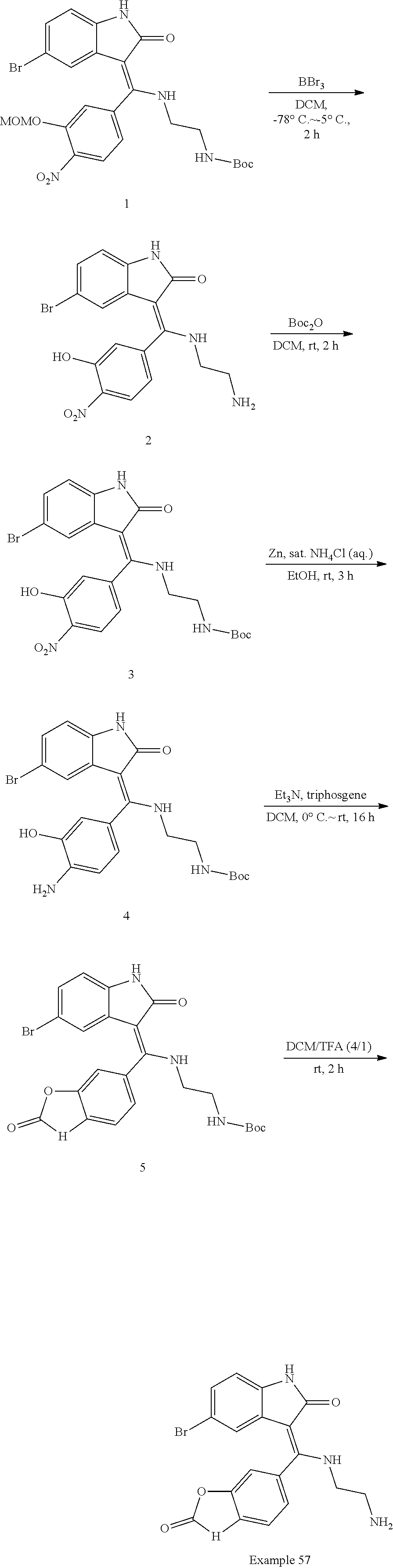

### Preparation of (Z)-3-(((2-aminoethyl)amino)(3-hydroxy-4-nitrophenyl)methylene)-5-bromoindolin-2-one

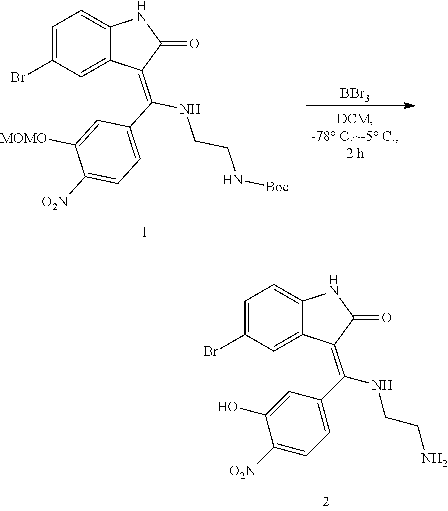

To a stirred solution of tert-butyl (Z)-(2-(((5-bromo-2-oxoindolin-3-ylidene)(3-(methoxy methoxy)-4-nitrophenyl)methyl) amino)ethyl)carbamate (500 mg, 0.89 mmol) in DCM (8 mL) was added BBr₃ (4.4 mL, 4.44 mmol, 1M in DCM) at −78° C. under nitrogen atmosphere. The resulting mixture was stirred for 2 h at −5° C. under nitrogen atmosphere. The reaction was quenched by the addition of sat. NaHCO₃ (aq.) (1 mL) and MeOH (1 mL) at −78° C. under nitrogen atmosphere. The resulting mixture was filtered, the filter cake was washed with EtOAc (3×20 mL). The resulting mixture was extracted with EtOAc (3×30 mL). The combined organic layers were washed with brine (2×20 mL), dried over anhydrous Na₂SO₄. After filtration, the filtrate was concentrated under reduced pressure. The residue was purified by reverse phase flash chromatography with the following conditions: Column: WelFlash TM C18-I, 20-40 μm, 330 g; Eluent A: Water (plus 0.05% TFA); Eluent B: ACN; Gradient: 5%-5% B in 1 min, 5%-50% B in 5 min, 50%-80% B in 10 min, 80%-95% B in 1 min, 95%-95% B in 3 min; Flow rate: 60 mL/min; Detector: UV 220/254 nm. The fractions containing desired product were collected at 75% B and concentrated under reduced pressure to afford (Z)-3-(((2-aminoethyl) amino)(3-hydroxy-4-nitrophenyl) methylene)-5-bromoindolin-2-one (250 mg, 68%) as a yellow solid: ¹H NMR (400 MHz, DMSO-d₆) δ 10.58 (s, 1H), 10.14 (t, J=6.4 Hz, 1H), 7.99 (d, J=8.2 Hz, 1H), 6.97 (dd, J=8.2, 2.1 Hz, 1H), 6.81-6.62 (m, 2H), 6.39-6.20 (m, 1H), 6.02 (s, 1H), 3.43-3.35 (m, 2H), 3.24-3.12 (m, 2H), 2.90 (t, J=7.0 Hz, 2H); LC/MS (ESI, m/z): [(M+1)]+=419.05.

### Preparation of Tert-butyl (Z)-(2-(((5-bromo-2-oxoindolin-3-ylidene)(3-hydroxy-4-nitrophenyl)methyl)amino)ethyl)carbamate

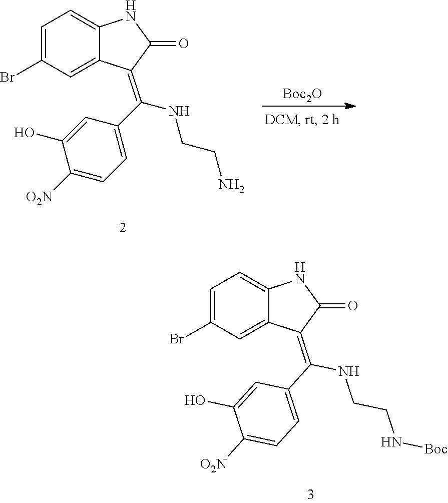 [SMILES: "Boc2O" ; NCCN/C(=C1\C(=O)Nc2ccc(Br)cc21)c1ccc([N+](=O)[O-])c(O)c1 ; CC(C)(C)OC(=O)NCCN/C(=C1\C(=O)Nc2ccc(Br)cc21)c1ccc([N+](=O)[O-])c(O)c1]

A solution of (Z)-3-(((2-aminoethyl)amino)(3-hydroxy-4-nitrophenyl)methylene)-5-bromoindolin-2-one (250 mg, 0.60 mmol) and Boc₂O (182 mg, 0.83 mmol) in MeOH (5 mL) was stirred for 2 h at room temperature under nitrogen atmosphere. The resulting mixture was concentrated under reduced pressure. The residue was purified by reverse phase flash chromatography with the following conditions: Column: WelFlash TM C18-I, 20-40 μm, 330 g; Eluent A: Water (plus 10 mM NH₄HCO₃); Eluent B: ACN; Gradient: 5%-50% B in 6 min, 50%-60% B in 1 min, 60%-80% B in 10 min, 80%-95% B in 10 min, 95%-95% B in 4 min; Flow rate: 50 mL/min; Detector: UV 220/254 nm. The fractions containing desired product were collected at 71% B and concentrated under reduced pressure to afford tert-butyl (Z)-(2-(((5-bromo-2-oxoindolin-3-ylidene) (3-hydroxy-4-nitrophenyl)methyl)amino)ethyl)carbamate (240 mg, 78%) as a brown solid: ¹H NMR (400 MHz, DMSO-d₆) δ 10.61 (s, 1H), 10.25 (t, J=6.4 Hz, 1H), 8.13 (d, J=8.3 Hz, 1H), 7.12 (s, 1H), 7.04-6.90 (m, 3H), 6.74 (d, J=8.2 Hz, 1H), 5.70 (d, J=2.0 Hz, 1H), 3.21-3.10 (m, 2H), 3.07-2.99 (m, 2H), 1.36 (s, 9H); LC/MS (ESI, m/z): [(M+1)]⁺=519.10.

### Preparation of Tert-butyl (Z)-(2-(((4-amino-3-hydroxyphenyl)(5-bromo-2-oxoindolin-3-ylidene)methyl)amino)ethyl)carbamate

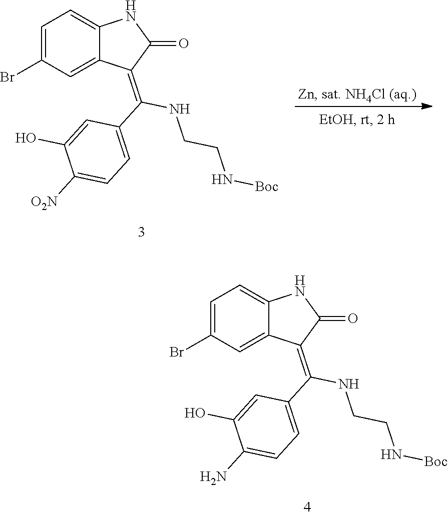 [SMILES: CC(C)(C)OC(=O)NCCN/C(=C1\C(=O)Nc2ccc(Br)cc21)c1ccc([N+](=O)[O-])c(O)c1 ; "Zn, sat. NH4Cl (aq.)" ; CC(C)(C)OC(=O)NCCN/C(=C1\C(=O)Nc2ccc(Br)cc21)c1ccc(N)c(O)c1]

A solution of tert-butyl (Z)-(2-(((5-bromo-2-oxoindolin-3-ylidene) (3-hydroxy-4-nitro phenyl)methyl)amino)ethyl)carbamate (240 mg, 0.46 mmol) and zinc powder (210 mg, 3.23 mmol) in sat. NH₄Cl (aq.) (2 mL) and EtOH (4 mL) was stirred for 2 h at room temperature under nitrogen atmosphere. The resulting mixture was filtered, the filter cake was washed with EtOH (3×5 mL). The filtrate was concentrated under reduced pressure. The residue was purified by reverse phase flash chromatography with the following conditions: Column: WelFlash TM C18-I, 20-40 μm, 120 g; Eluent A: Water (plus 10 mM NH₄HCO₃); Eluent B: ACN; Gradient: 5%-5% B in 1 min, 5%-50% B in 5 min, 50%-80% B in 10 min, 80%-95% B in 2 min, 95%-95% B in 4 min; Flow rate: 50 mL/min; Detector: UV 220/254 nm. The fractions containing desired product were collected at 78% B and concentrated under reduced pressure to afford tert-butyl (Z)-(2-(((4-amino-3-hydroxyphenyl)(5-bromo-2-oxoindolin-3-ylidene)methyl)amino)ethyl) carbamate (190 mg, 85%) as a brown solid. ¹H NMR (400 MHz, DMSO-d₆) δ 10.44 (s, 1H), 10.25 (t, J=6.3 Hz, 1H), 9.44 (s, 1H), 6.97 (s, 1H), 6.91 (dd, J=8.2, 2.0 Hz, 1H), 6.79 (d, J=7.9 Hz, 1H), 6.70 (d, J=8.2 Hz, 1H), 6.62 (d, J=1.9 Hz, 1H), 6.57 (dd, J=7.9, 1.9 Hz, 1H), 5.95 (d, J=2.0 Hz, 1H), 5.02 (s, 2H), 3.24-3.15 (m, 2H), 3.06-2.99 (m, 2H), 1.37 (s, 9H); LC/MS (ESI, m/z): [(M+1)]⁺=489.10.

### Preparation of Tert-butyl(Z)-(2-(((5-bromo-2-oxoindolin-3-ylidene)(2-oxo-2,3-dihydrobenzo[d]oxazol-6-yl)methyl)amino)ethyl) carbamate

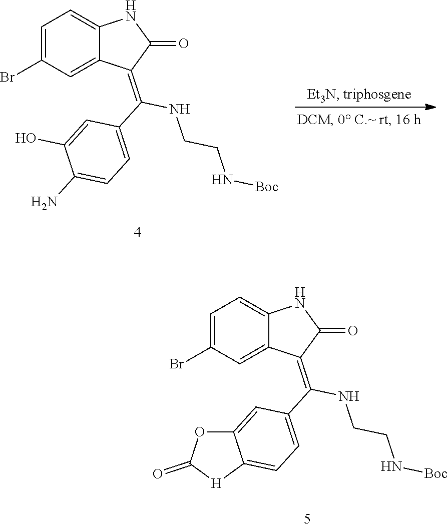

To a stirred solution of tert-butyl (Z)-(2-(((4-amino-3-hydroxyphenyl)(5-bromo-2-oxoindolin-3-ylidene) methyl)amino)ethyl)carbamate (200 mg, 0.41 mmol) and Et₃N (83 mg, 0.82 mmol) in DCM (4 mL) was added triphosgene (73 mg, 0.25 mmol) at 0° C. under nitrogen atmosphere. The resulting mixture was stirred for 16 h at room temperature under nitrogen atmosphere. The reaction was quenched by the addition of sat. NH₄HCO₃ (aq.) (1 mL) at 0° C. under nitrogen atmosphere. The resulting mixture was concentrated under reduced pressure. The residue was purified by reverse phase flash chromatography with the following conditions: Column: WelFlash TM C18-I, 20-40 μm, 330 g; Eluent A: Water (plus 10 mM NH₄HCO₃); Eluent B: ACN; Gradient: 5%-50% B in 6 min, 50%-60% B in 1 min, 60%-80% B in 10 min, 80%-95% B in 10 min, 95%-95% B in 4 min; Flow rate: 50 mL/min; Detector: UV 220/254 nm. The fractions containing desired product were collected at 72% B and concentrated under reduced pressure to afford crude tert-butyl (Z)-(2-(((5-bromo-2-oxoindolin-3-ylidene)(2-oxo-2,3-dihydrobenzo[d]oxazol-6-yl)methyl)amino)ethyl)carbamate (66 mg, 32%) as a yellow oil: LC/MS (ESI, m/z): [(M+1)]⁺=515.10.

### Preparation of (Z)-6-(((2-aminoethyl)amino)(5-bromo-2-oxoindolin-3-ylidene) methyl)benzo[d]oxazol-2(3H)-one (Example 57)

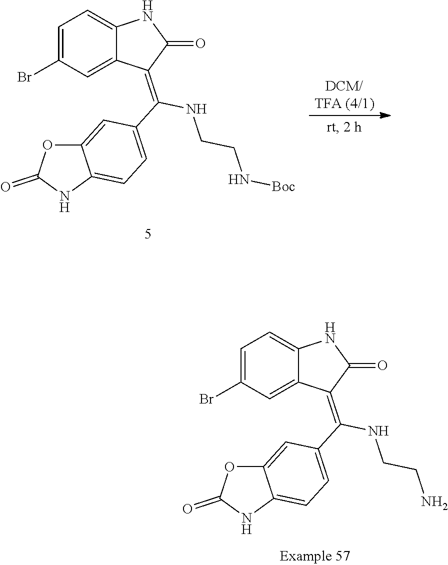 [SMILES: CC(C)(C)OC(=O)NCCN/C(=C1\C(=O)Nc2ccc(Br)cc21)c1ccc2[nH]c(=O)oc2c1 ; NCCN/C(=C1\C(=O)Nc2ccc(Br)cc21)c1ccc2[nH]c(=O)oc2c1]

A solution of tert-butyl (Z)-(2-(((5-bromo-2-oxoindolin-3-ylidene)(2-oxo-2,3-dihydrobenzo[d]oxazol-6-yl)methyl)amino)ethyl)carbamate (60 mg, 0.12 mmol) in TFA (0.2 mL) and DCM (0.8 mL) was stirred for 2 h at room temperature under nitrogen atmosphere. The resulting mixture was concentrated under reduced pressure. The residue was purified by reverse phase flash chromatography with the following conditions: Column: WelFlash TM C18-I, 20-40 μm, 330 g; Eluent A: Water (plus 10 mM NH₄HCO₃); Eluent B: ACN; Gradient: 5%-30% B in 6 min, 30%-60% B in 10 min, 60%-80% B in 1 min, 80%-95% B in 1 min, 95%-95% B in 4 min; Flow rate: 50 mL/min; Detector: UV 220/254 nm. The fractions containing desired product were collected at 47% B and concentrated under reduced pressure to afford (Z)-6-(((2-aminoethyl) amino)(5-bromo-2-oxoindolin-3-ylidene)methyl)benzo[d]oxazol-2(3H)-one (20 mg, 42%) as a yellow solid: ¹H NMR (400 MHz, DMSO-d₆) δ 10.65 (s, 1H), 10.18 (t, J=6.6 Hz, 1H), 7.53 (d, J=1.5 Hz, 1H), 7.41 (d, J=7.9 Hz, 1H), 7.25 (dd, J=7.9, 1.5 Hz, 1H), 6.97 (dd, J=8.2, 2.0 Hz, 1H), 6.74 (d, J=8.2 Hz, 1H), 5.52 (d, J=1.9 Hz, 1H), 3.38-3.29 (m, 2H), 2.91 (t, J=6.6 Hz, 2H); LC/MS (ESI, m/z): [(M+1)]⁺=415.10.

### Example 58: (Z)-3-((3-aminophenyl)((3-fluoropropyl)amino)methylene)-5-bromoindolin-2-one

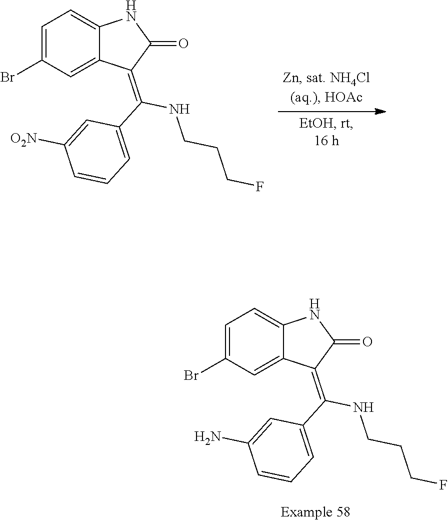 [SMILES: O=C1Nc2ccc(Br)cc2/C1=C(/NCCCF)c1cccc([N+](=O)[O-])c1 ; Nc1cccc(/C(NCCCF)=C2/C(=O)Nc3ccc(Br)cc32)c1]

Example 58 was prepared the same manner as the preparation of compound 4 above (in the preparation of Example 57) using (Z)-5-bromo-3-(((3-fluoropropyl)amino)(3-nitrophenyl) methylene)indolin-2-one (100 mg, 0.24 mmol) as starting material to give the title compound (55 mg, 60%) as a yellow solid: ¹H NMR (400 MHz, DMSO-d₆) δ 10.52 (s, 1H), 10.24 (t, J=6.3 Hz, 1H), 7.28 (t, J=7.8 Hz, 1H), 6.92 (dd, J=8.2, 2.1 Hz, 1H), 6.81-6.77 (m, 1H), 6.71 (d, J=8.2 Hz, 1H), 6.53 (t, J=1.9 Hz, 1H), 6.48 (dt, J=7.6, 1.2 Hz, 1H), 5.79 (d, J=2.0 Hz, 1H), 5.45 (s, 2H), 4.53 (t, J=5.7 Hz, 1H), 4.41 (t, J=5.7 Hz, 1H), 3.29-3.21 (m, 2H), 1.94-1.81 (m, 2H); LC/MS (ESI, m/z): [(M+1)]⁺=390.05.

### Example 59: (Z)-3-(((1H-pyrazol-4-yl)amino)(3-aminophenyl)methylene)-5-bromoindolin-2-one

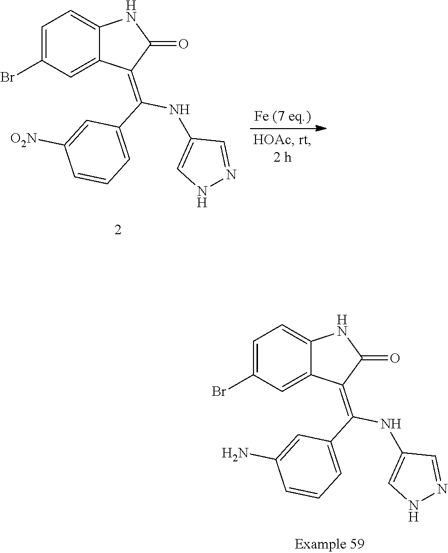 [SMILES: O=C1Nc2ccc(Br)cc2/C1=C(/Nc1cn[nH]c1)c1cccc([N+](=O)[O-])c1 ; Nc1cccc(/C(Nc2cn[nH]c2)=C2/C(=O)Nc3ccc(Br)cc32)c1]

To a stirred solution of (Z)-3-(((1H-pyrazol-4-yl)amino)(3-nitrophenyl)methylene)-5-bromoindolin-2-one (200 mg, 0.47 mmol) in HOAc (10 mL) was added iron powder (458 mg, 8.21 mmol) in portions at room temperature under nitrogen atmosphere. The resulting mixture was stirred for 2 h at room temperature under nitrogen atmosphere. The reaction was monitored by LCMS. The resulting mixture was filtered, the filter cake was washed with DCM (3×30 mL). The filtrate was concentrated under reduced pressure. The residue was basified to pH 8 with saturated NaHCO₃ (aq.). The residue was purified by reverse flash chromatography with the following conditions: Column: Spherical C18, 20-40 μm, 120 g; Eluent A: Water (plus 0.1% HCOOH), Eluent B: ACN; Flow rate: 50 mL/min; Gradient (B %): 5%-25%, 6 min; 25%-50%, 4 min; 50%-65%, 3 min; 65%-80%, 12 min; 80%-95%, 1 min; Detector: UV 220 nm. The fractions containing desired product were collected at 56% B and concentrated under reduced pressure to afford (Z)-3-(((1H-pyrazol-4-yl)amino)(3-aminophenyl)methylene)-5-bromoindolin-2-one (80 mg, 44%) as a yellow solid: ¹H NMR (400 MHz, DMSO-d₆) δ 12.62 (s, 1H), 11.78 (s, 1H), 10.71 (s, 1H), 7.27 (t, J=8.0 Hz, 1H), 7.16 (s, 1H), 7.00 (dd, J=8.2, 2.0 Hz, 2H), 6.84-6.72 (m, 2H), 6.56-6.52 (m, 2H), 5.99 (d, J=2.0 Hz, 1H), 5.42 (s, 2H); LC/MS (ESI, m/z): [(M+1)]⁺=396.05.

### Example 60 was prepared using the procedure of Example 59

| Ex. | | | MS: | |
|---|---|---|---|---|
| No. | Structure | Name | [(M + 1)]⁺ | ¹H NMR |
| 60 | 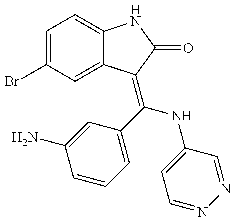 [SMILES: Nc1cccc(/C(Nc2ccnnc2)=C2/C(=O)Nc3ccc(Br)cc32)c1] | (Z)-3-((3-aminophenyl) (pyridazin-4-ylamino) methylene)-5-bromo indolin-2-one | 410.05 | ¹H NMR (400 MHz, DMSO-d₆) δ 11.87 (s, 1H), 11.02 (s, 1H), 8.79 (dd, J = 6.1, 1.0 Hz, 1H), 8.72 (dd, J = 3.0, 1.0 Hz, 1H), 7.33 (t, J = 7.7 Hz, 1H), 7.16 (dd, J = 8.2, 2.0 Hz, 1H), 6.91- 6.84 (m, 1H), 6.82 (d, J = 8.2 Hz, 1H), 6.69-6.61 (m, 3H), 6.22 (d, J = 2.0 Hz, 1H), 5.55 (s, 2H). |

### Example 61: (Z)—N-(1-(5-bromo-2-oxoindolin-3-ylidene)-1-(3-hydroxyphenyl)-5,8,11-trioxa-2-azatridecan-13-yl)acetamide

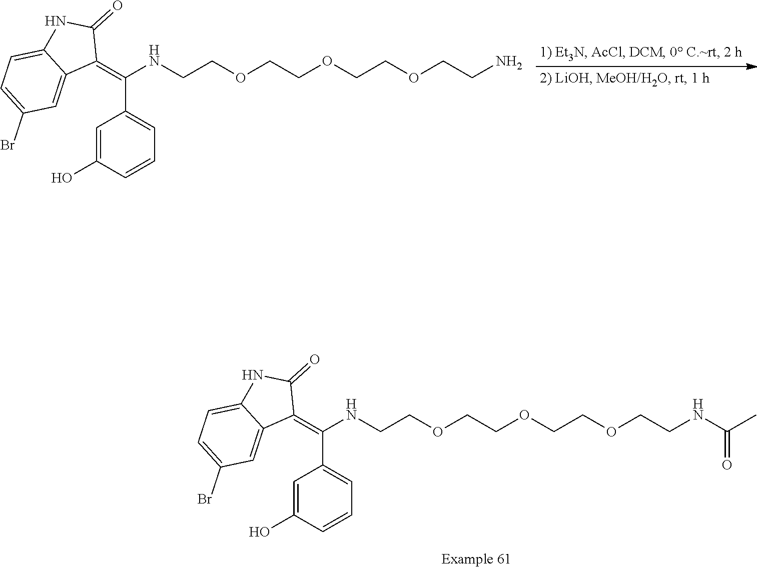 [SMILES: NCCOCCOCCOCCN/C(=C1\C(=O)Nc2ccc(Br)cc21)c1cccc(O)c1 ; CC(=O)NCCOCCOCCOCCN/C(=C1\C(=O)Nc2ccc(Br)cc21)c1cccc(O)c1]

To a stirred solution of (Z)-3-(13-amino-1-(3-hydroxyphenyl)-5,8,11-trioxa-2-azatridecylidene)-5-bromoindolin-2-one (120 mg, 0.24 mmol) and Et₃N (48 mg, 0.47 mmol) in DCM (3 mL) was added AcCl (25 mg, 0.31 mmol) dropwise at 0° C. under nitrogen atmosphere. The resulting mixture was stirred for 2 h at room temperature under nitrogen atmosphere. The resulting mixture was concentrated under reduced pressure. The residue was dissolved in MeOH (2 mL) and sat. LiGH (aq.) (0.5 mL) at room temperature. The resulting mixture was stirred for additional 1 h at room temperature. The solution was purified by reverse phase flash chromatography with the following conditions: Column: WelFlash TM C18-I, 20-40 μm, 330 g; Eluent A: Water (plus 10 mM NH₄HCO₃); Eluent B: ACN; Gradient: 5%-25% B in 4 min, 25%-75% B in 8 min, 75%-95% B in 2 min, 85%-95% B in 2 min, 95%-95% B in 3 min; Flow rate: 80 mL/min; Detector: UV 220/254 nm; The fractions containing desired product were collected at 41% B and concentrated under reduced pressure to afford (Z)—N-(1-(5-bromo-2-oxoindolin-3-ylidene)-1-(3-hydroxyphenyl)-5,8,11-trioxa-2-azatridecan-13-yl)acetamide (81 mg, 62%) as a yellow solid. ¹H NMR (400 MHz, DMSO-d₆) δ 10.52 (s, 1H), 10.31 (t, J=6.2 Hz, 1H), 7.88 (s, 1H), 7.45 (t, J=7.9 Hz, 1H), 7.02 (dd, J=8.4, 2.5 Hz, 1H), 6.93 (dd, J=8.2, 2.0 Hz, 1H), 6.81 (d, J=7.5 Hz, 1H), 6.75 (t, J=2.0 Hz, 1H), 6.71 (d, J=8.2 Hz, 1H), 5.63 (d, J=2.0 Hz, 1H), 3.57-3.44 (m, 10H), 3.40-3.34 (m, 2H), 3.28-3.21 (m, 2H), 3.19-3.12 (m, 2H), 1.78 (s, 3H); LC/MS (ESI, m/z): [(M+1)]⁺=548.15.

### Examples 62-64

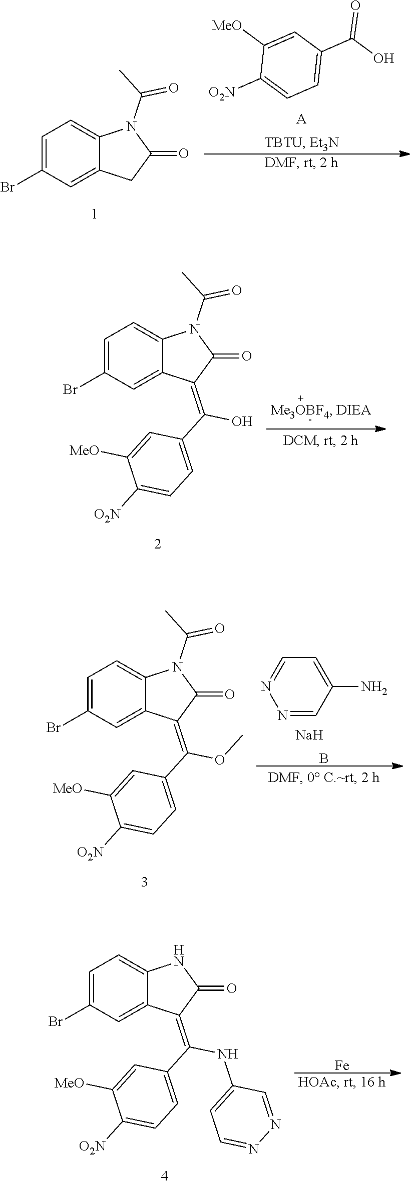

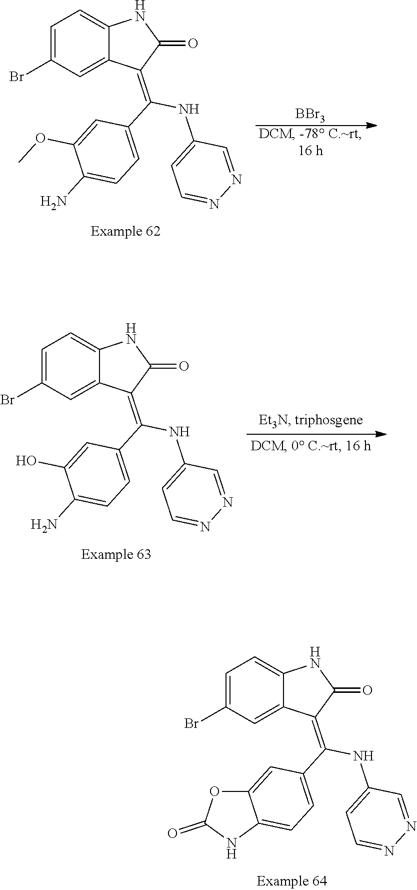

### Preparation of (Z)-1-acetyl-5-bromo-3-(hydroxy(3-methoxy-4-nitrophenyl)methylene) indolin-2-one

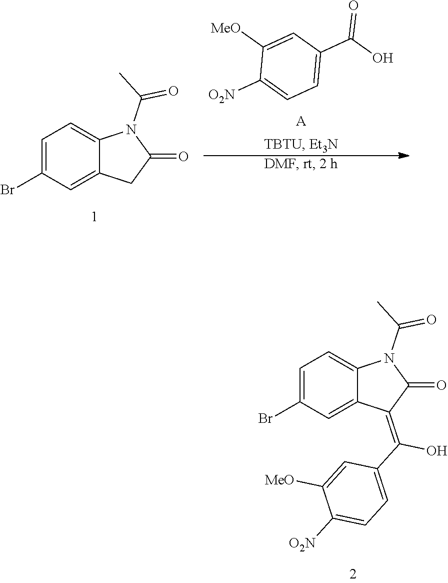

To a stirred solution of 1-acetyl-5-bromoindolin-2-one (2.00 g, 7.88 mmol) and 3-methoxy-4-nitrobenzoic acid (2.02 g, 10.24 mmol) in DMF (30 mL) were added TBTU (3.79 g, 11.81 mmol) and Et₃N (2.39 g, 23.64 mmol) dropwise at room temperature. The resulting mixture was stirred for 2 h at room temperature. The resulting mixture was diluted with water (200 mL). The resulting mixture was extracted with EtOAc (3×150 mL). The combined organic layers were washed with brine (2×100 mL), dried over anhydrous Na₂SO₄. After filtration, the filtrate was concentrated under reduced pressure. The residue was purified by silica gel column chromatography, eluted with CH₂Cl₂/MeOH (100:1 to 15:1) to afford (Z)-1-acetyl-5-bromo-3-(hydroxy(3-methoxy-4-nitrophenyl)methylene)indolin-2-one (2.2 g, 65%) as a yellow green solid: ¹H NMR (400 MHz, CDCl₃) δ 13.91 (s, 1H), 8.24 (d, J=8.8 Hz, 1H), 8.04 (d, J=8.3 Hz, 1H), 7.53-7.43 (m, 2H), 7.40 (dd, J=8.8, 2.0 Hz, 1H), 7.29 (d, J=2.0 Hz, 1H), 4.05 (s, 3H), 2.83 (s, 3H); LC/MS (ESI, m/z): [(M+1)]⁺=432.95.

### Preparation of (Z)-1-acetyl-5-bromo-3-(methoxy(3-methoxy-4-nitrophenyl)methylene) indolin-2-one

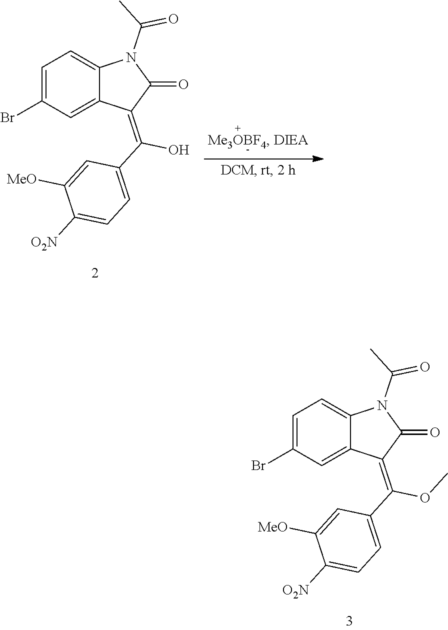 [SMILES: COc1cc(/C(O)=C2/C(=O)N(C(C)=O)c3ccc(Br)cc32)ccc1[N+](=O)[O-] ; CO/C(=C1\C(=O)N(C(C)=O)c2ccc(Br)cc21)c1ccc([N+](=O)[O-])c(OC)c1]

To a stirred solution of (Z)-1-acetyl-5-bromo-3-(hydroxy(3-methoxy-4-nitrophenyl) methylene)indolin-2-one (2.20 g, 5.08 mmol) and trimethyloxonium tetrafluoroborate (3.76 g, 25.39 mmol) in DCM (30 mL) was added DIEA (2.63 g, 20.32 mmol) dropwise at room temperature. The resulting mixture was stirred for 2 h at room temperature. The resulting mixture was concentrated under reduced pressure. The residue was purified by silica gel column chromatography, eluted with petroleum ether/EtOAc (20:1 to 4:1) to afford (Z)-1-acetyl-5-bromo-3-(methoxy(3-methoxy-4-nitrophenyl)methylene)indolin-2-one (925 mg, 41%) as a yellow solid: ¹H NMR (400 MHz, CDCl₃) δ 8.17 (d, J=8.7 Hz, 1H), 8.05 (d, J=2.1 Hz, 1H), 8.02 (d, J=8.2 Hz, 1H), 7.42 (dd, J=8.7, 2.2 Hz, 1H), 7.10-7.03 (m, 2H), 4.00 (s, 3H), 3.79 (s, 3H), 2.54 (s, 3H); LC/MS (ESI, m/z): [(M+1)]⁺=447.00.

### Preparation of (Z)-5-bromo-3-((3-methoxy-4-nitrophenyl)(pyridazin-4-ylamino) methylene)indolin-2-one

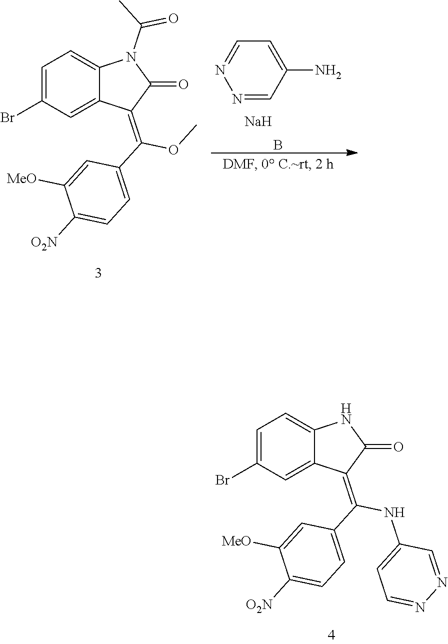

A solution of 4-pyridazinamine (984 mg, 10.35 mmol) in DMF (15 mL) was treated with NaH (331 mg, 8.28 mmol, 60%, mineral oil) for 30 min at 0° C. under nitrogen atmosphere followed by the addition of (Z)-1-acetyl-5-bromo-3-(methoxy(3-methoxy-4-nitrophenyl) methylene)indolin-2-one (925 mg, 2.07 mmol) in portions at room temperature. The resulting mixture was stirred for 2 h at room temperature under nitrogen atmosphere. The reaction was quenched with sat. NH₄Cl (aq.) at 0° C. The resulting mixture was purified by reverse phase flash chromatography with the following conditions: Column: Spherical C18, 20-40 μm, 330 g; Eluent A: Water (plus 10 mM NH₄HCO₃); Eluent B: ACN; Flow rate: 80 mL/min; Gradient (B %): 5%-5%, 4 min, 5%-45%, 4 min, 45%-70%, 30 min; Detector: UV 220 nm. The fractions containing the desired product were collected at 61% and concentrated under reduced pressure to afford (Z)-5-bromo-3-((3-methoxy-4-nitrophenyl)(pyridazin-4-ylamino)methylene)indolin-2-one (505 mg, 53%) as an orange oil: ¹H NMR (400 MHz, DMSO-d₆) δ 11.89 (s, 1H), 11.11 (s, 1H), 8.85-8.79 (m, 2H), 8.18 (d, J=8.2 Hz, 1H), 7.75 (d, J=1.6 Hz, 1H), 7.36 (dd, J=8.2, 1.6 Hz, 1H), 7.21 (dd, J=8.3, 2.0 Hz, 1H), 6.86 (d, J=8.3 Hz, 1H), 6.70 (dd, J=5.9, 3.1 Hz, 1H), 6.02 (d, J=2.0 Hz, 1H), 3.89 (s, 3H). LC/MS (ESI, m/z): [(M+1)]⁺=468.00.

### Preparation of (Z)-3-((4-amino-3-methoxyphenyl)(pyridazin-4-ylamino)methylene)-5-bromoindolin-2-one (Example 62)

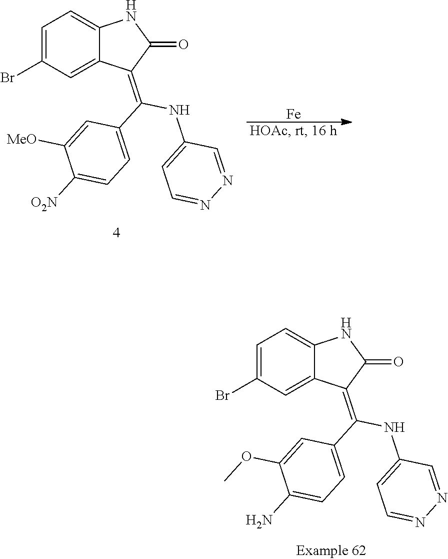

A mixture of (Z)-5-bromo-3-((3-methoxy-4-nitrophenyl)(pyridazin-4-ylamino) methylene)indolin-2-one (440 mg, 0.94 mmol) and iron powder (526 mg, 9.40 mmol) in AcOH (10 mL) was stirred for 16 h at room temperature. The resulting mixture was filtered, the filter cake was washed with EtOAc (2×20 mL). The filtrate was concentrated under reduced pressure. The residue was neutralized to pH 7 with saturated NaHCO₃ (aq.). The resulting mixture was purified by reverse phase flash chromatography with the following conditions: Column: Spherical C18, 20-40 μm, 330 g; Eluent A: Water (plus 10 mM NH₄HCO₃); Eluent B: ACN; Flow rate: 80 mL/min; Gradient (B %): 5%-5%, 4 min, 5%-25%, 3 min, 25%-35%, 3 min, 35%-50% gradient in 20 min; Detector: UV 220 nm. The fractions containing the desired product were collected at 48% and concentrated under reduced pressure to afford crude product which was further purified by Prep-HPLC with the following conditions: Column: XBridge Shield RP18 OBD Column, 30*150 mm, 5 m; Eluent A: Water (10 mmol/L NH₄HCO₃), Eluent B: ACN; Flow rate: 60 mL/min; Gradient (B %): 36% to 46%, 8 min; Detector: UV 254 nm; RT1: 6.5 min. Desired fractions were collected and concentrated under reduced pressure to afford (Z)-3-((4-amino-3-methoxyphenyl)(pyridazin-4-ylamino)methylene)-5-bromoindolin-2-one (251 mg, 61%) as a yellow solid: ¹H NMR (400 MHz, DMSO-d₆) δ 11.57 (s, 1H), 10.94 (s, 1H), 8.79 (d, J=6.0 Hz, 1H), 8.69 (d, J=2.8 Hz, 1H), 7.16 (dd, J=8.2, 2.0 Hz, 1H), 6.95 (d, J=1.8 Hz, 1H), 6.89-6.77 (m, 3H), 6.66 (dd, J=6.0, 2.9 Hz, 1H), 6.58 (d, J=2.0 Hz, 1H), 5.59 (s, 2H), 3.70 (s, 3H); LC/MS (ESI, m/z): [(M+1)]⁺=438.05.

### Preparation of (Z)-3-((4-amino-3-hydroxyphenyl)(pyridazin-4-ylamino)methylene)-5-bromoindolin-2-one (Example 63)

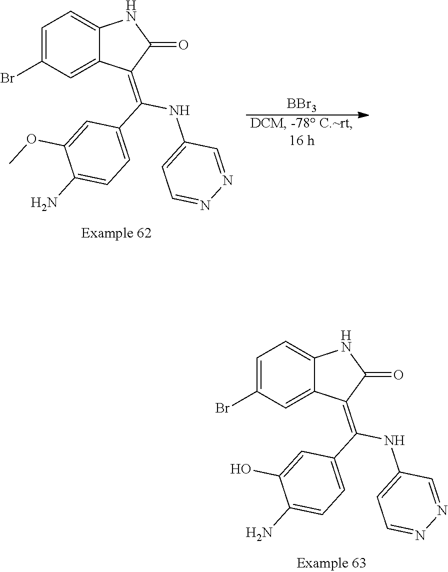

To a stirred solution of (Z)-3-((4-amino-3-methoxyphenyl)(pyridazin-4-ylamino) methylene)-5-bromoindolin-2-one (200 mg, 0.46 mmol) in DCM (4 mL) was added BBr₃ (4.56 mL, 4.56 mmol, 1 M in DCM) dropwise at −78° C. under nitrogen atmosphere. The resulting mixture was stirred for 16 h at room temperature under nitrogen atmosphere. The mixture was neutralized to pH 7 with saturated Na₂CO₃ (aq.) and was quenched with MeOH (2 mL) at −78° C. The resulting mixture was purified by reverse phase flash chromatography with the following conditions: Column: Spherical C18, 20-40 μm, 330 g; Eluent A: Water (plus 10 mM NH₄HCO₃); Eluent B: ACN; Flow rate: 80 mL/min; Gradient (B %): 5%-5%, 4 min, 30%-60%, 20 min; Detector: UV 220 nm. The fractions containing the desired product were collected at 48% and concentrated under reduced pressure to afford (Z)-3-((4-amino-3-hydroxyphenyl)(pyridazin-4-ylamino)methylene)-5-bromoindolin-2-one (140 mg, 72%) as a yellow solid: ¹H NMR (400 MHz, DMSO-d₆) δ 11.53 (s, 1H), 10.92 (s, 1H), 9.60 (s, 1H), 8.83-8.77 (m, 1H), 8.68 (dd, J=2.9, 1.0 Hz, 1H), 7.15 (dd, J=8.3, 2.0 Hz, 1H), 6.85-6.73 (m, 3H), 6.70 (d, J=1.8 Hz, 1H), 6.65 (dd, J=6.0, 2.9 Hz, 1H), 6.58 (d, J=2.0 Hz, 1H), 5.36 (s, 2H); LC/MS (ESI, m/z): [(M+1)]⁺=424.05.

### Preparation of (Z)-6-((5-bromo-2-oxoindolin-3-ylidene)(pyridazin-4-ylamino)methyl) benzo[d]oxazol-2(3H)-one (Example 64)

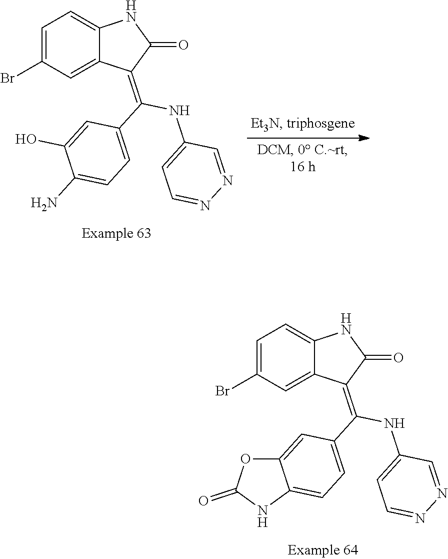 [SMILES: Nc1ccc(/C(Nc2ccnnc2)=C2/C(=O)Nc3ccc(Br)cc32)cc1O ; O=C1Nc2ccc(Br)cc2/C1=C(/Nc1ccnnc1)c1ccc2[nH]c(=O)oc2c1]

To a stirred solution of (Z)-3-((4-amino-3-hydroxyphenyl)(pyridazin-4-ylamino) methylene)-5-bromoindolin-2-one (60 mg, 0.15 mmol) and TEA (29 mg, 0.29 mmol) in DCM (1 mL) was added triphosgene (26 mg, 0.09 mmol) dropwise at 0° C. under nitrogen atmosphere. The resulting mixture was stirred for 16 h at room temperature under nitrogen atmosphere. The mixture was neutralized to pH 7 with saturated NaHCO₃ (aq.). The resulting mixture was concentrated under reduced pressure. The resulting mixture was purified by reverse phase flash chromatography with the following conditions: Column: Spherical C18, 20-40 μm, 120 g; Eluent A: Water (plus 10 mM NH₄HCO₃); Eluent B: ACN; Flow rate: 60 mL/min; Gradient (B %): 5%-5%, 4 min, 5%-35%, 4 min, 35%-55% 15 min; Detector: UV 220 nm. The fractions containing the desired product were collected at 50% and concentrated under reduced pressure to afford (Z)-6-((5-bromo-2-oxoindolin-3-ylidene)(pyridazin-4-ylamino)methyl)benzo[d]oxazol-2(3H)-one (39 mg, 62%) as a yellow solid: ¹H NMR (400 MHz, DMSO-d₆) δ 12.13 (s, 1H), 11.80 (s, 1H), 11.06 (s, 1H), 8.75 (t, J=4.9 Hz, 2H), 7.60 (s, 1H), 7.38 (s, 2H), 7.18 (dd, J=8.3, 2.0 Hz, 1H), 6.84 (d, J=8.2 Hz, 1H), 6.54 (dd, J=6.0, 3.0 Hz, 1H), 6.08 (d, J=1.9 Hz, 1H); LC/MS (ESI, m/z): [(M+1)]⁺=450.00.

### Example 65: (Z)-5-bromo-3-((3-hydroxyphenyl)(phenylamino)methylene)indolin-2-one

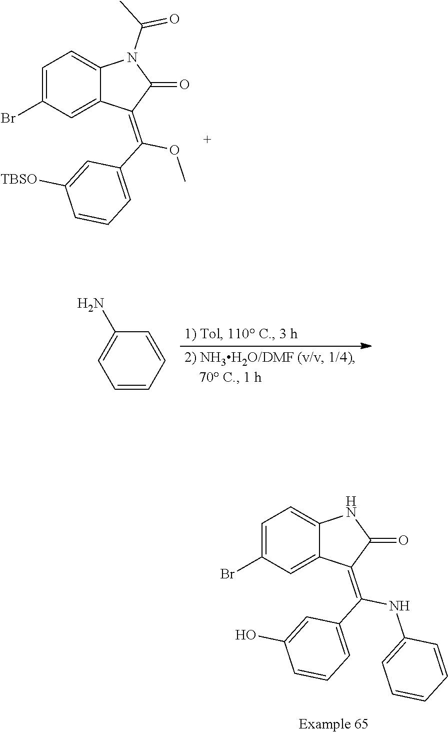 [SMILES: CO/C(=C1\C(=O)N(C(C)=O)c2ccc(Br)cc21)c1cccc(O[Si](C)(C)C(C)(C)C)c1 ; Nc1ccccc1 ; O=C1Nc2ccc(Br)cc2/C1=C(/Nc1ccccc1)c1cccc(O)c1]

A mixture of (Z)-1-acetyl-5-bromo-3-((3-((tert-butyldimethylsilyl)oxy)phenyl) (methoxy) methylene)indolin-2-one (80 mg, 0.16 mmol) and aniline (45 mg, 0.48 mmol) in toluene (1.5 mL) was stirred for 3 h at 110° C. under nitrogen atmosphere. The mixture was allowed to cool down to room temperature. The resulting mixture was concentrated under reduced pressure. The residue was dissolved in DMF (2.4 mL) and NH₃·H₂O (0.6 mL) at room temperature. The resulting mixture was stirred for 1 h at 70° C. under nitrogen atmosphere. The mixture was allowed to cool down to room temperature. The resulting mixture was purified by reverse phase flash with the following conditions: Column: Spherical C18, 20-40 μm, 120 g; Eluent A: Water (plus 10 mM NH₄HCO₃), Eluent B: ACN; Flow rate: 50 mL/min; Gradient (B %): 5%-22%, 4 min; 22%-40%, 20 min; 40%-95%; 2 min; 95%, 5 min; Detector: UV 254 nm.) The fractions containing desired product were collected at 56% B and concentrated under reduced pressure to afford ((Z)-5-bromo-3-((3-hydroxyphenyl)(phenylamino)methylene)indolin-2-one (50 mg, 77%) as a yellow solid: ¹H NMR (400 MHz, DMSO-d₆) δ 12.04 (s, 1H), 10.83 (s, 1H), 9.85 (s, 1H), 7.40 (t, J=7.9 Hz, 1H), 7.24-7.15 (m, 2H), 7.08-6.96 (m, 3H), 6.94-6.84 (m, 3H), 6.83-6.77 (m, 2H), 5.93 (d, J=2.0 Hz, 1H); LC/MS (ESI, m/z): [(M+1)]⁺=407.00.

The following exemplary compounds were prepared using the procedure of Example 65

| Ex. | | | MS: | |
|---|---|---|---|---|
| No. | Structure | Name | [(M + 1)]⁺ | ¹H NMR |
| 66 | 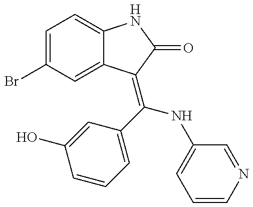 [SMILES: O=C1Nc2ccc(Br)cc2/C1=C(/Nc1cccnc1)c1cccc(O)c1] | (Z)-5-bromo-3-((3- hydroxyphenyl) (pyridin-3-ylamino) methylene)indolin- 2-one | 408.05 | ¹H NMR (400 MHz, DMSO- d₆) δ 11.91 (s, 1H), 10.87 (s, 1H), 9.87 (s, 1H), 8.25-8.16 (m, 2H), 7.40 (t, J = 7.8 Hz, 1H), 7.29-7.17 (m, 2H), 7.08 (dd, J = 8.3, 2.0 Hz, 1H), 6.98 (dd, J = 8.3, 2.5 Hz, 1H), 6.91 (dt, J = 7.5, 1.2 Hz, 1H), 6.84- 6.77 (m, 2H), 5.97 (d, J = 2.0 Hz, 1H). |
| 67 | 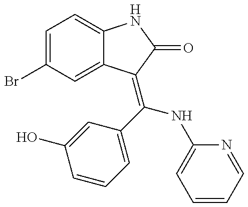 [SMILES: O=C1Nc2ccc(Br)cc2/C1=C(/Nc1ccccn1)c1cccc(O)c1] | (Z)-5-bromo-3-((3- hydroxyphenyl) (pyridin-2-ylamino) methylene)indolin-2- one | 408.05 | ¹H NMR (400 MHz, DMSO- d₆) δ 12.24 (s, 1H), 10.90 (s, 1H), 9.96 (s, 1H), 8.27 (dd, J = 5.2, 1.8 Hz, 1H), 7.55-7.44 (m, 2H), 7.09 (td, J = 9.2, 8.8, 2.3 Hz, 2H), 7.00 (dd, J = 7.3, 4.8 Hz, 1H), 6.96 (dt, J = 7.5, 1.2 Hz, 1H), 6.86 (t, J = 2.0 Hz, 1H), 6.80 (d, J = 8.2 Hz, 1H), 6.22 (d, J = 8.4 Hz, 1H), 5.92 (d, J = 1.9 Hz, 1H). |
| 68 | 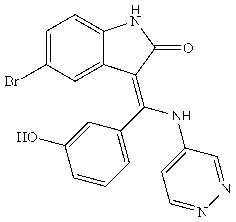 [SMILES: O=C1Nc2ccc(Br)cc2/C1=C(/Nc1ccnnc1)c1cccc(O)c1] | (Z)-5-bromo-3-((3- hydroxyphenyl) (pyridazin-4-ylamino) methylene)indolin-2- one | 409.05 | ¹H NMR (400 MHz, DMSO- d₆) δ 11.87 (s, 1H), 11.04 (s, 1H), 10.04 (s, 1H), 8.82-8.76 (m, 1H), 8.74-8.69 (m, 1H), 7.51 (t, J = 7.9 Hz, 1H), 7.16 (dd, J = 8.3, 2.0 Hz, 1H), 7.14- 7.07 (m, 1H), 7.03-6.97 (m, 1H), 6.93-6.87 (m, 1H), 6.83 (d, J = 8.2 Hz, 1H), 6.61 (dd, J = 6.0, 3.0 Hz, 1H), 6.06 (d, J = 2.0 Hz, 1H). |
| 69 | 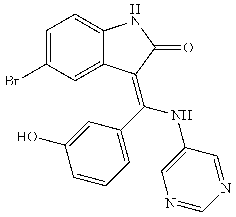 [SMILES: O=C1Nc2ccc(Br)cc2/C1=C(/Nc1cncnc1)c1cccc(O)c1] | (Z)-5-bromo-3-((3- hydroxyphenyl) (pyrimidin-5-ylamino) methylene)indolin-2- one | 409.05 | ¹H NMR (400 MHz, DMSO- d₆) δ 11.76 (s, 1H), 10.94 (s, 1H), 9.95 (s, 1H), 8.81 (s, 1H), 8.36 (s, 2H), 7.41 (t, J = 7.8 Hz, 1H), 7.11 (d, J = 7.8 Hz, 1H), 7.03-6.96 (m, 1H), 6.93 (d, J = 7.6 Hz, 1H), 6.86-6.78 (m, 2H), 5.99 (d, J = 2.0 Hz, 1H). |
| 70 | 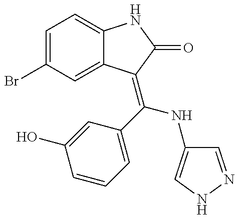 [SMILES: O=C1Nc2ccc(Br)cc2/C1=C(/Nc1cn[nH]c1)c1cccc(O)c1] | (Z)-3-(((1H-pyrazol- 4-yl)amino)(3- hydroxyphenyl) methylene)-5- bromoindolin-2-one | 397.00 | ¹H NMR (400 MHz, DMSO- d₆) δ 12.63 (s, 1H), 11.73 (s, 1H), 10.75 (s, 1H), 9.87 (s, 1H), 7.44 (t, J = 7.8 Hz, 1H), 7.21 (s, 1H), 7.69-7.04 (m, 2H), 6.89-6.86 (m, 1H), 6.81- 6.74 (m, 2H), 5.84 (d, J = 2.0 Hz, 1H). |
| 71 | 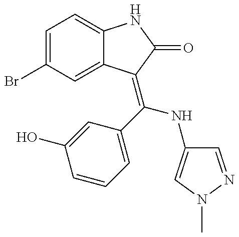 [SMILES: Cn1cc(N/C(=C2\C(=O)Nc3ccc(Br)cc32)c2cccc(O)c2)cn1] | (Z)-5-bromo-3-((3- hydroxyphenyl)((1- methyl-1H-pyrazol- 4-yl)amino) methylene)indolin-2- one | 411.05 | ¹H NMR (400 MHz, DMSO- d₆) δ 11.70 (s, 1H), 10.77 (s, 1H), 9.89 (s, 1H), 7.43 (t, J = 7.9 Hz, 1H), 7.29 (s, 1H), 7.04-6.99 (m, 2H), 6.90-6.80 (m, 2H), 6.80-6.73 (m, 2H), 5.81 (d, J = 2.0 Hz, 1H), 3.67 (s, 3H). |
| 72 | 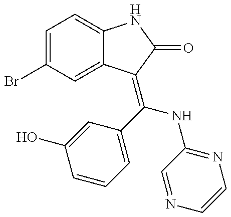 [SMILES: O=C1Nc2ccc(Br)cc2/C1=C(/Nc1cnccn1)c1cccc(O)c1] | (Z)-5-bromo-3-((3- hydroxyphenyl) (pyrazin-2-ylamino) methylene)indolin-2- one | 409.05 | ¹H NMR (400 MHz, DMSO- d₆) δ 12.23 (s, 1H), 11.00 (s, 1H), 10.02 (s, 1H), 8.31 (dd, J = 2.6, 1.5 Hz, 1H), 8.21 (d, J = 2.6 Hz, 1H), 7.55 (d, J = 1.5 Hz, 1H), 7.50 (t, J = 7.9 Hz, 1H), 7.14 (dd, J = 8.3, 2.0 Hz, 1H), 7.12-7.06 (m, 1H), 6.99 (d, J = 7.5 Hz, 1H), 6.91 (t, J = 2.0 Hz, 1H), 6.83 (d, J = 8.3 Hz, 1H), 6.01 (d, J = 2.0 Hz, 1H). |
| 73 | 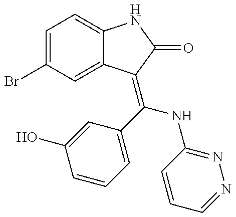 [SMILES: O=C1Nc2ccc(Br)cc2/C1=C(/Nc1cccnn1)c1cccc(O)c1] | (Z)-5-bromo-3-((3- hydroxyphenyl) (pyridazin-3-ylamino) methylene)indolin-2- one | 411.00 | ¹H NMR (400 MHz, DMSO- d₆) δ 12.51 (s, 1H), 11.00 (s, 1H), 10.10 (s, 1H), 8.84 (dd, J = 4.8, 1.2 Hz, 1H), 7.48 (t, J = 8.0 Hz, 1H), 7.41 (dd, J = 9.2, 4.8 Hz, 1H), 7.14 (dd, J = 8.4, 2.0 Hz, 1H), 7.08 (dd, J = 8.4, 2.4 Hz, 1H), 6.97 (d, J = 7.6 Hz, 1H), 6.89 (t, J = 2.0 Hz, 1H), 6.83 (d, J = 8.4 Hz, 1H), 6.47 (dd, J = 9.2, 1.2 Hz, 1H), 6.00 (d, J = 2.0 Hz, 1H). |

### Example 74: (Z)-4-(((5-bromo-2-oxoindolin-3-ylidene)(3-hydroxyphenyl)methyl) amino)benzoic acid

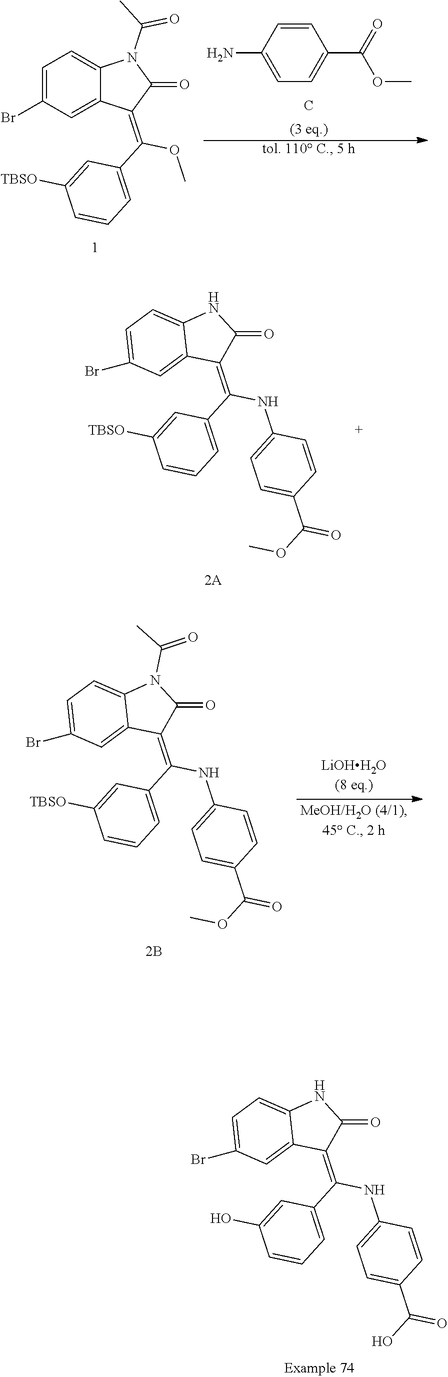

### Preparation of Methyl (Z)-4-(((1-acetyl-5-bromo-2-oxoindolin-3-ylidene)(3-((tert-butyldimethylsilyl)oxy)phenyl)methyl)amino)benzoate

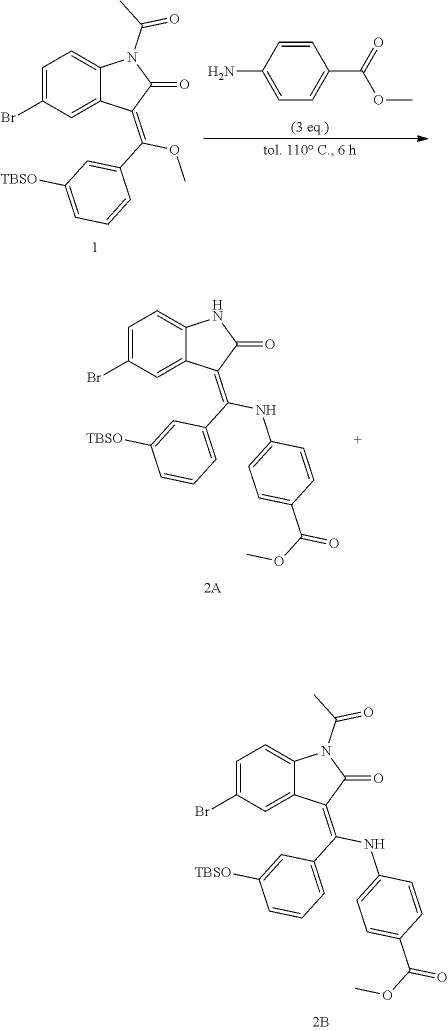

A mixture of (Z)-1-acetyl-5-bromo-3-((3-((tert-butyldimethylsilyl)oxy)phenyl)(methoxy) methylene)indolin-2-one (350 mg, 0.70 mmol) and methyl 4-aminobenzoate (316 mg, 2.09 mmol) in toluene (7 mL) was stirred for 6 h at 110° C. under nitrogen atmosphere. The mixture was allowed to cool down to room temperature. The resulting mixture was concentrated under reduced pressure. The residue was purified by Prep-TLC (petroleum/EtOAc, 3.5/1) to afford methyl (Z)-4-(((5-bromo-2-oxoindolin-3-ylidene)(3-((tert-butyldimethylsilyl)oxy)phenyl) methyl)amino)benzoate (90 mg, 23%) as an orange solid: ¹H NMR (400 MHz, DMSO-d₆) δ 11.79 (s, 1H), 8.08 (d, J=8.7 Hz, 1H), 7.79-7.74 (m, 2H), 7.53 (t, J=7.9 Hz, 1H), 7.23 (dd, J=8.7, 2.1 Hz, 1H), 7.18-7.12 (m, 2H), 7.07-7.03 (m, 2H), 6.93 (t, J=2.0 Hz, 1H), 5.87 (d, J=2.1 Hz, 1H), 3.80 (s, 3H), 0.89 (s, 9H), 0.11 (s, 3H), 0.09 (s, 3H); LC/MS (ESI, m/z): [(M+1)]⁺=579.20. Chemical Formula: C29H31BrN204Si, Exact Mass: 578.12. and methyl (Z)-4-(((1-acetyl-5-bromo-2-oxoindolin-3-ylidene)(3-((tert-butyldimethylsilyl)oxy)phenyl)methyl)amino) benzoate (108 mg, 25%) as a yellow solid: ¹H NMR (400 MHz, DMSO-d₆) δ 7.92-7.88 (m, 2H), 7.73 (d, J=4.9 Hz, 1H), 7.54 (t, J=7.9 Hz, 1H), 7.20-7.12 (m, 2H), 7.09 (dd, J=8.2, 2.0 Hz, 1H), 6.94 (t, J=2.0 Hz, 1H), 6.92-6.85 (m, 2H), 6.81 (d, J=8.3 Hz, 1H), 5.92 (d, J=2.0 Hz, 1H), 3.82 (s, 3H), 2.08 (s, 3H), 0.90 (s, 9H), 0.12 (s, 3H), 0.10 (s, 3H); LC/MS (ESI, m/z): [(M+1)]⁺=621.25.

### Preparation of (Z)-4-(((5-bromo-2-oxoindolin-3-ylidene)(3-hydroxyphenyl)methyl) amino)benzoic acid (Example 74)

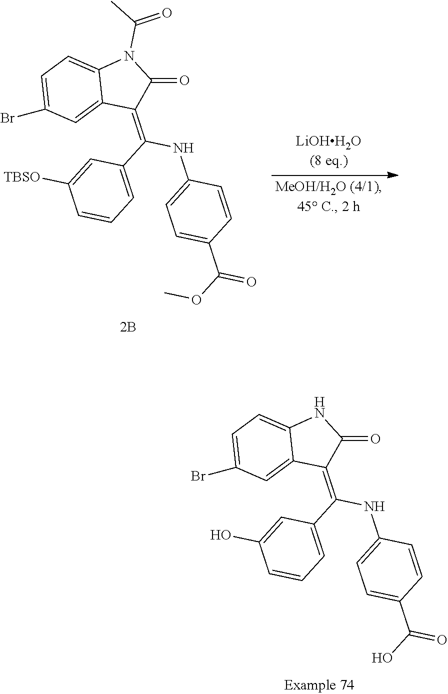 [SMILES: COC(=O)c1ccc(N/C(=C2\C(=O)N(C(C)=O)c3ccc(Br)cc32)c2cccc(O[Si](C)(C)C(C)(C)C)c2)cc1 ; O=C1Nc2ccc(Br)cc2/C1=C(/Nc1ccc(C(=O)O)cc1)c1cccc(O)c1]

A mixture of (Z)-4-(((1-acetyl-5-bromo-2-oxoindolin-3-ylidene)(3-((tert-butyldimethyl silyl)oxy)phenyl)methyl)amino)benzoate (105 mg, 0.17 mmol) and LiGH (33 mg, 1.35 mmol) in MeOH (4 mL) and H₂O (1 mL) was stirred for 2 h at 45° C. under nitrogen atmosphere. The mixture was allowed to cool down to room temperature. The mixture was purified by reverse flash chromatography with the following conditions: Column: Spherical C18, 20-40 μm, 120 g; Eluent A: Water (0.1% HCOOH), Eluent B: ACN; Flow rate: 50 mL/min; Gradient (B %): 5%-30%, 7 min; 30%-45%, 8 min; 45%-57%; 4 min; 57%-95%, 3 min; 95%, 3 min; Detector: UV 254 nm. The fractions containing desired product were collected at 57% B and concentrated under reduced pressure to afford (Z)-4-(((5-bromo-2-oxoindolin-3-ylidene)(3-hydroxyphenyl) methyl)amino)benzoic acid (40 mg, 53%) as a yellow solid: ¹H NMR (400 MHz, DMSO-d₆) δ 12.12 (s, 1H), 10.91 (s, 1H), 10.06 (br, 1H), 7.74-7.67 (m, 2H), 7.44 (t, J=7.9 Hz, 1H), 7.09 (dd, J=8.3, 2.0 Hz, 1H), 7.03 (dd, J=8.1, 2.5 Hz, 1H), 6.98-6.91 (m, 1H), 6.89-6.78 (m, 4H), 5.99 (d, J=2.0 Hz, 1H); LC/MS (ESI, m/z): [(M+1)]⁺=451.05.

### General Synthesis of Indolinones with NH from Thioamide

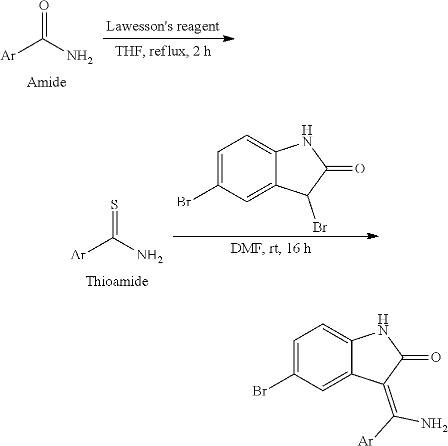

### General Synthesis of Thioamide

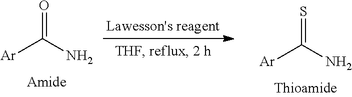

General Procedure: A solution of Amide (1 mmol) and Lawesson's reagent (0.6 mmol) in THF (3 mL) was refluxed for 2 h under nitrogen atmosphere. After cooling down to ambient temperature, the resulting mixture was concentrated under reduced pressure. The residue was purified by silica gel chromatography. Desired fractions were collected and concentrated under reduced pressure to afford the Desired Thioamide.

The following thioamides were prepared using the General Synthesis of Thioamide as above from the corresponding amide.

| | | MS: | |
|---|---|---|---|
| Structure | Name | [(M + 1)]⁺ | ¹H NMR |
| 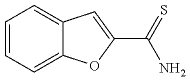 [SMILES: NC(=S)c1cc2ccccc2o1] | benzofuran-2- carbothioamid | 178.20 | ¹H NMR (400 MHz, CDCl₃) δ 7.81 (d, J = 0.7 Hz, 1H), 7.75 (s, 1H), 7.68 (dt, J = 7.9, 1.0 Hz, 1H), 7.53-7.35 (m, 3H), 7.33-7.28 (m, 1H). |

### Preparation of 2-Aminothiazole-4-carbothioamide

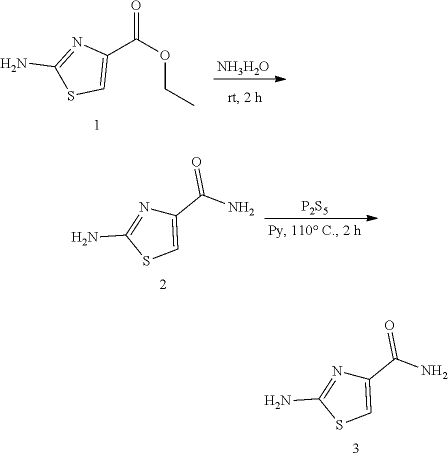

### Preparation of 2-Aminothiazole-4-carboxamide

A mixture of ethyl 2-aminothiazole-4-carboxylate (3 g, 17.42 mmol) in NH₃H₂O (100 mL) was stirred for 2 h at room temperature under nitrogen atmosphere. The resulting mixture was concentrated under reduced pressure. The residue was purified by silica gel column chromatography, eluted with 0-100% EtOAc in petroleum ether to afford 2-aminothiazole-4-carboxamide (1.8 g, 73%) as a yellow solid: ¹H NMR (400 MHz, DMSO-d₆) δ 7.32 (s, 1H), 7.17 (s, 1H), 7.09-7.00 (m, 3H); LC/MS (ESI, m/z): [(M+1)]⁺=144.15.

### Preparation of 2-Aminothiazole-4-carbothioamide

A solution of 2-aminothiazole-4-carboxamide (200 mg, 1.40 mmol) and P₂S₅(559 mg, 2.51 mmol) in pyridine (4 mL) was stirred for 2 h at 110° C. under nitrogen atmosphere. The mixture was allowed to cool down to room temperature. The resulting mixture was concentrated under reduced pressure. The residue was purified by silica gel column chromatography, eluted with 0-100% EtOAc in petroleum ether to afford 2-aminothiazole-4-carbothioamide (160 mg, 72%) as a yellow solid: ¹H NMR (400 MHz, CDCl₃) δ 8.36 (br, 1H), 7.75 (s, 1H), 7.38 (s, 1H), 4.92 (s, 2H). LC/MS (ESI, m/z): [(M+1)]⁺=160.10.

### Preparation of 7-Methoxy-3,4-dihydroisoquinoline-1(2H)-thione

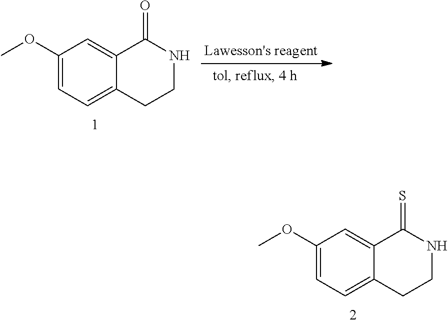

To a stirred solution of 7-methoxy-3,4-dihydro-2H-isoquinolin-1-one (500 mg, 2.82 mmol) in methylbenzene (10 mL) was added Lawesson's reagent (684 mg, 1.69 mmol) in portions at room temperature under nitrogen atmosphere. The resulting mixture was stirred for 4 h at 110° C. under nitrogen atmosphere. The resulting mixture was concentrated under reduced pressure. The residue was purified by silica gel column chromatography, eluted with petroleum ether/EtOAc (6:1 to 1:1) to afford 7-methoxy-3,4-dihydroisoquinoline-1(2H)-thione (520 mg, 95%) as a yellow solid: ¹H NMR (400 MHz, DMSO-d₆) δ 10.47 (s, 1H), 7.88 (d, J=2.8 Hz, 1H), 7.20 (d, J=8.3 Hz, 1H), 7.07 (dd, J=8.3, 2.9 Hz, 1H), 3.78 (s, 3H), 3.41-3.32 (m, 2H), 2.84 (t, J=6.9 Hz, 2H); LC/MS (ESI, m/z): [(M+1)]⁺=194.05.

### Preparation of 5-(Methoxymethoxy)pyridine-3-carbothioamide (5)

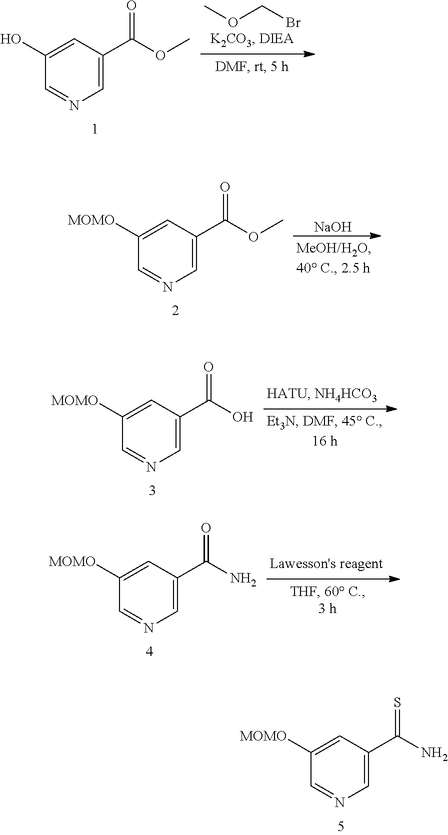

### Preparation of Methyl 5-(methoxymethoxy)nicotinate

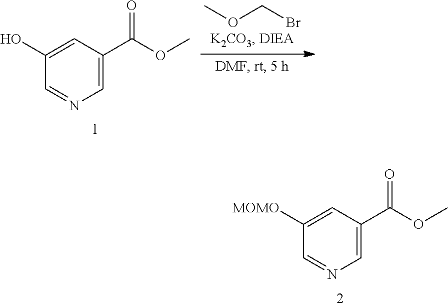

To a stirred mixture of methyl 5-hydroxypyridine-3-carboxylate (6.0 g, 39.18 mmol), DIEA (10.13 g, 78.36 mmol) and K₂CO₃ (5.4 g, 39.18 mmol) in DMF (60 mL) was added bromo(methoxy)methane (9.80 g, 78.42 mmol) dropwise over 3 min at 0° C. The resulting mixture was stirred for additional 5 h at room temperature. The resulting mixture was diluted with water (480 mL). The resulting mixture was extracted with EtOAc (3×150 mL). The combined organic layers were washed with brine (3×100 mL), dried over anhydrous Na₂SO₄. After filtration, the filtrate was concentrated under reduced pressure. The residue was purified by silica gel column chromatography, eluted with petroleum ether/EtOAc (10:1 to 1:1) to afford methyl 5-(methoxymethoxy)pyridine-3-carboxylate (7.11 g, 92%) as a light yellow solid: H NMR (400 MHz, CDCl₃) δ 8.89 (d, J=1.7 Hz, 1H), 8.57 (d, J=2.8 Hz, 1H), 7.96 (dd, J=2.9, 1.8 Hz, 1H), 5.26 (s, 2H), 3.96 (s, 3H), 3.51 (s, 3H); LC/MS (ESI, m/z): [(M+1)]⁺=198.25.

### Preparation of 5-(Methoxymethoxy)nicotinic acid

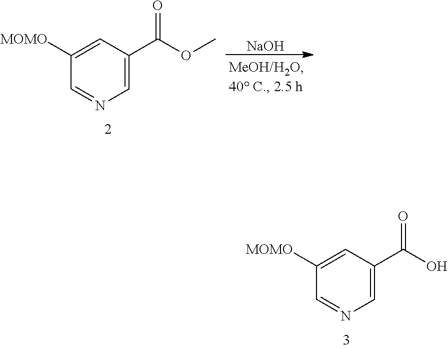

To a stirred solution of methyl 5-(methoxymethoxy)pyridine-3-carboxylate (4.00 g, 20.28 mmol) in MeOH (40 mL) and H₂O (2 mL) was added NaOH (1.62 g, 40.51 mmol) in portions at room temperature under nitrogen atmosphere. The resulting mixture was stirred for 2.5 h at 40° C. under nitrogen atmosphere. After cooling down The mixture was acidified to pH 6 with 2 M HCl (aq.). The resulting mixture was concentrated under reduced pressure to afford crude 5-(methoxymethoxy)nicotinic acid (3.2 g, 87%) as an off-white solid which was used in the next step directly without further purification: ¹H NMR (400 MHz, DMSO-d₆) δ 8.68 (d, J=1.6 Hz, 1H), 8.28 (d, J=2.9 Hz, 1H), 7.83-7.81 (m, 1H), 5.25 (s, 2H), 3.39 (s, 3H); LC/MS (ESI, m/z): [(M+1)]⁺=183.95.

### Preparation of 5-(Methoxymethoxy)nicotinamide

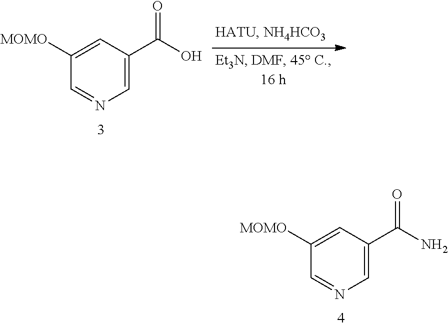

To a stirred mixture of 5-(methoxymethoxy)pyridine-3-carboxylic acid (500 mg, 2.73 mmol) and Et₃N (1.14 mL, 8.19 mmol) in DMF (10 mL) were added HATU (2.59 g, 6.82 mmol) and NH₄HCO₃ (1.08 g, 13.65 mmol) in portions at room temperature. The resulting mixture was stirred for 16 h at 45° C. The mixture was allowed to cool down to room temperature. The resulting mixture was purified by reverse phase flash chromatography with the following conditions: Column: Spherical C18, 20-40 μm, 330 g; Eluent A: Water (plus 10 mM NH₄HCO₃); Eluent B: ACN; Flow rate: 80 mL/min; Gradient: 5%-5% B, 10 min, 5% B-15% B gradient in 20 min; Detector: UV 220 nm. The fractions containing the desired product were collected at 10% B and concentrated under reduced pressure to afford 5-(methoxymethoxy)pyridine-3-carboxamide (327 mg, 66%) as a white solid: ¹H NMR (400 MHz, DMSO-d₆) δ 8.70 (d, J=1.8 Hz, 1H), 8.47 (d, J=2.8 Hz, 1H), 8.15 (s, 1H), 7.87-7.85 (m, 1H), 7.62 (s, 1H), 5.32 (s, 2H), 3.42 (s, 3H); LC/MS (ESI, m/z): [(M+1)]⁺=183.30.

### Preparation of 5-(Methoxymethoxy)pyridine-3-carbothioamide

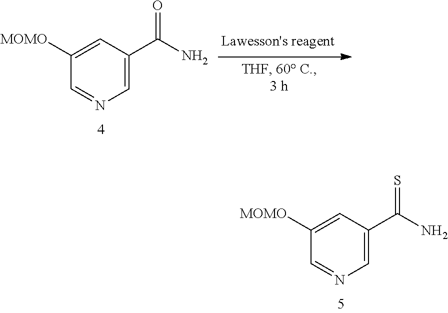

A mixture of 5-(methoxymethoxy)pyridine-3-carboxamide (540 mg, 2.96 mmol) and Lawesson Reagent (720 mg, 1.78 mmol) in THF (10 mL) was stirred for 3 h at 60° C. The mixture was allowed to cool down to room temperature. The resulting mixture was concentrated under reduced pressure. The residue was purified by reverse phase flash chromatography with the following conditions: Column: Spherical C18, 20-40 μm, 330 g; Eluent A: Water (plus 10 mM NH₄HCO₃); Eluent B: ACN; Flow rate: 80 mL/min; Gradient: 5%-5% B, 10 min, 5% B-25% B gradient in 20 min; Detector: UV 220 nm. The fractions containing the desired product were collected at 15% B and concentrated under reduced pressure to afford 5-(methoxymethoxy) pyridine-3-carbothioamide (182 mg, 31%) as an off-white solid: ¹H NMR (400 MHz, DMSO-d₆) δ 10.10 (s, 1H), 9.71 (s, 1H), 8.65 (d, J=1.9 Hz, 1H), 8.44 (d, J=2.7 Hz, 1H), 7.87-7.83 (m, 1H), 5.31 (s, 2H), 3.41 (s, 3H); LC/MS (ESI, m/z): [(M+1)]⁺=199.25.

### Preparation of 5-Bromo-4-methoxypyridine-2-carbothioamide

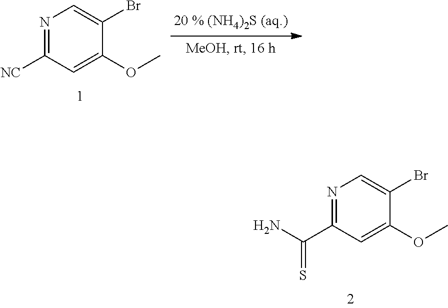

To a stirred solution of 5-bromo-4-methoxypyridine-2-carbonitrile (300 mg, 1.41 mmol) in MeOH (3 mL) was added 20% (NH₄)₂S (aq.) (465 mg) dropwise at room temperature under nitrogen atmosphere. The resulting mixture was stirred for 16 h at room temperature under nitrogen atmosphere. The resulting mixture was concentrated under reduced pressure to give crude 5-bromo-4-methoxypyridine-2-carbothioamide (240 mg, 69%) as a yellow solid which was used in the next step directly without further purification: ¹H NMR (400 MHz, CDCl₃) δ 9.42 (s, 1H), 8.49 (s, 1H), 8.31 (s, 1H), 7.66 (s, 1H), 4.09 (s, 3H); LC/MS (ESI, m/z): [(M+1)]⁺=246.80.

### Preparation of Tert-butyl (2-(5-amino-2-fluorophenylthioamido)ethyl)carbamate

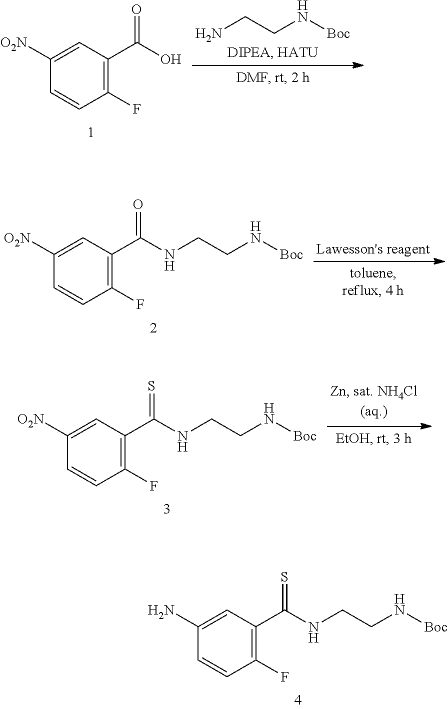 [SMILES: O=C(O)c1cc([N+](=O)[O-])ccc1F ; CC(C)(C)OC(=O)NCCN ; CC(C)(C)OC(=O)NCCNC(=O)c1cc([N+](=O)[O-])ccc1F ; CC(C)(C)OC(=O)NCCNC(=S)c1cc([N+](=O)[O-])ccc1F ; CC(C)(C)OC(=O)NCCNC(=S)c1cc(N)ccc1F]

### Preparation of Tert-butyl (2-(2-fluoro-5-nitrobenzamido)ethyl)carbamate

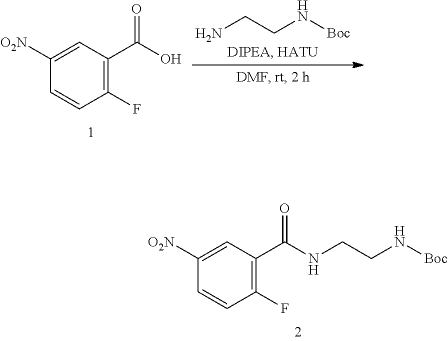 [SMILES: O=C(O)c1cc([N+](=O)[O-])ccc1F ; CC(C)(C)OC(=O)NCCN ; CC(C)(C)OC(=O)NCCNC(=O)c1cc([N+](=O)[O-])ccc1F]

To a stirred solution of 2-fluoro-5-nitrobenzoic acid (5.00 g, 27.01 mmol), DIEA (6.98 g, 54.02 mmol) and tert-butyl N-(2-aminoethyl)carbamate (6.49 g, 40.51 mmol) in DMF (60 mL) was added HATU (15.41 g, 40.51 mmol) in portions at 0° C. under nitrogen atmosphere. The resulting mixture was stirred for 2 h at room temperature under nitrogen atmosphere. The solution was purified by reverse phase flash chromatography with the following conditions: Column: WelFlash TM C18-I, 20-40 μm, 330 g; Eluent A: Water (plus 10 mM NH₄HCO₃); Eluent B: ACN; Gradient: 5%-25% B in 4 min, 25%-75% B in 2 min, 75%-95% B in 8 min, 85%-95% B in 2 min, 95%-95% B in 3 min; Flow rate: 80 mL/min; Detector: UV 220/254 nm. The fractions containing desired product were collected at 77% B and concentrated under reduced pressure to afford tert-butyl (2-(2-fluoro-5-nitrobenzamido)ethyl)carbamate (1.6 g, 18%) as a yellow solid: ¹H NMR (400 MHz, CDCl₃) δ 8.95 (dd, J=6.4, 3.0 Hz, 1H), 8.37-8.33 (m, 1H), 7.38 (s, 1H), 7.34-7.27 (m, 1H), 3.65-3.59 (m, 2H), 3.45-3.39 (m, 2H), 1.43 (s, 9H); LC/MS (ESI, m/z): [(M−1)]⁻=326.10.

### Preparation of Tert-butyl (2-(2-fluoro-5-nitrophenylthioamido)ethyl)carbamate

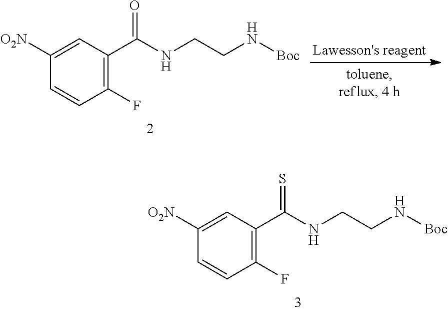 [SMILES: CC(C)(C)OC(=O)NCCNC(=O)c1cc([N+](=O)[O-])ccc1F ; CC(C)(C)OC(=O)NCCNC(=S)c1cc([N+](=O)[O-])ccc1F]

A solution of tert-butyl (2-(2-fluoro-5-nitrobenzamido)ethyl)carbamate (1.90 g, 5.80 mmol) and Lawesson Reagent (1.41 g, 3.483 mmol) in toluene (25 mL) was refluxed for 4 h under nitrogen atmosphere. After cooling down to room temperature, the resulting mixture was concentrated under reduced pressure. The residue was purified by silica gel column chromatography, eluted with petroleum ether/EtOAc (5:1 to 1:1) to afford tert-butyl (2-(2-fluoro-5-nitrophenylthioamido)ethyl)carbamate (600 mg, 30%) as a yellow solid: ¹H NMR (400 MHz, CDCl₃) δ 9.40 (s, 1H), 8.80 (dd, J=6.4, 2.9 Hz, 1H), 8.29-8.21 (m, 1H), 7.25-7.20 (m, 1H), 5.05 (s, 1H), 3.92-3.84 (m, 2H), 3.58-3.51 (m, 2H), 1.41 (s, 9H); LC/MS (ESI, m/z): [(M+1)]⁺=344.30.

### Preparation of Tert-butyl (2-(5-amino-2-fluorophenylthioamido)ethyl)carbamate

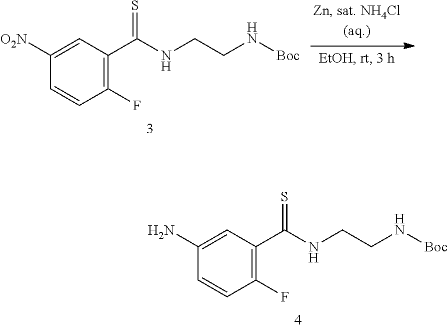 [SMILES: CC(C)(C)OC(=O)NCCNC(=S)c1cc([N+](=O)[O-])ccc1F ; CC(C)(C)OC(=O)NCCNC(=S)c1cc(N)ccc1F]

A mixture of tert-butyl (2-(2-fluoro-5-nitrophenylthioamido)ethyl)carbamate (600 mg, 1.74 mmol) and Zn (1.14 g, 17.44 mmol) in sat. NH₄Cl (5 mL) and EtOH (10 mL) was stirred for 3 h at room temperature under nitrogen atmosphere. The resulting mixture was filtered, the filter cake was washed with EtOH (3×10 mL). The filtrate was concentrated under reduced pressure. The residue was purified by silica gel column chromatography, eluted with petroleum ether/EtOAc (5:1 to 1:1) to afford tert-butyl (2-(5-amino-2-fluorophenylthioamido)ethyl)carbamate (470 mg, 86%) as a yellow solid: ¹H NMR (400 MHz, CDCl₃) δ 8.84 (s, 1H), 7.36 (s, 1H), 6.86 (dd, J=11.0, 8.7 Hz, 1H), 6.69 (s, 1H), 4.93 (s, 1H), 3.96-3.85 (m, 2H), 3.56-3.44 (m, 2H), 3.02 (br, 2H), 1.43 (s, 9H); LC/MS (ESI, m/z): [(M+1)]⁺=314.15.

### Preparation of 4-(difluoromethyl)pyridine-2-carbothioamide

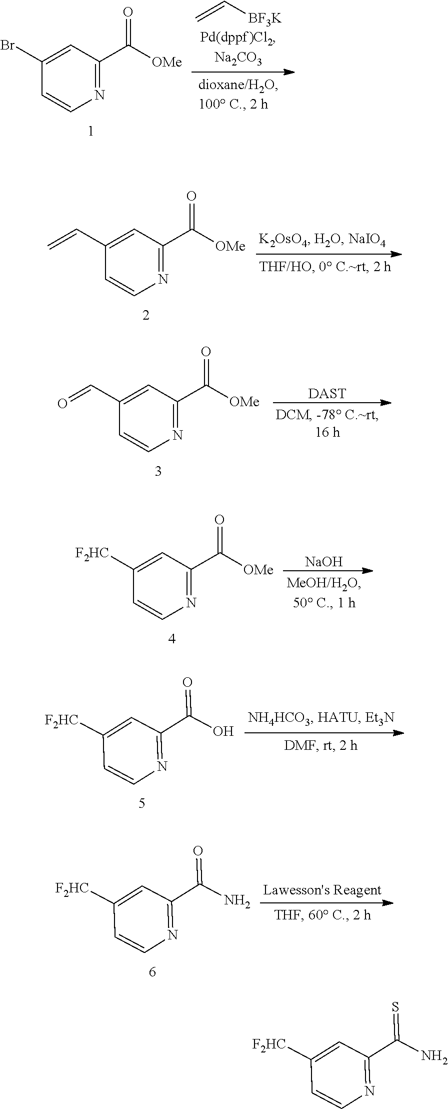

### Preparation of Methyl 4-vinylpicolinate

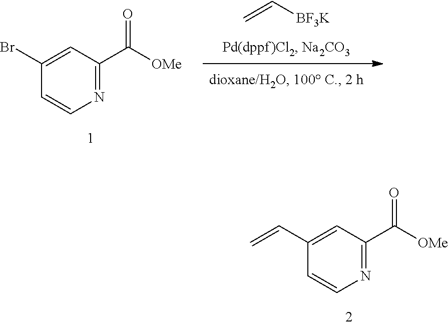

To a stirred solution of methyl 4-bromopyridine-2-carboxylate (10.0 g, 46.28 mmol) and potassium ethenyldifluoroborane fluoride (12.4 g, 92.57 mmol) in 1,4-dioxane (160 mL) and H₂O (40 mL) were added Pd(dppf)Cl₂ (3.4 g, 4.62 mmol) and Na₂CO₃ (9.8 g, 92.57 mmol) in portions at room temperature under nitrogen atmosphere. The resulting mixture was stirred for 2 h at 100° C. under nitrogen atmosphere. The reaction was monitored by LCMS. The resulting mixture was concentrated under reduced pressure. The residue was purified by silica gel column chromatography, eluted with petroleum ether/EtOAc (2:1 to 1:1) to afford methyl 4-vinylpicolinate (6.4 g, 85%) as an orange oil: ¹H NMR (400 MHz, CDCl₃) δ 8.69 (d, J=5.0 Hz, 1H), 8.14 (d, J=1.8 Hz, 1H), 7.44 (dd, J=5.1, 1.8 Hz, 1H), 6.73 (dd, J=17.6, 10.8 Hz, 1H), 6.08 (d, J=17.5 Hz, 1H), 5.59 (d, J=10.9 Hz, 1H), 4.02 (s, 3H); LC/MS (ESI, m/z): [(M+1)]⁺=165.05.

### Preparation of Methyl 4-formylpicolinate

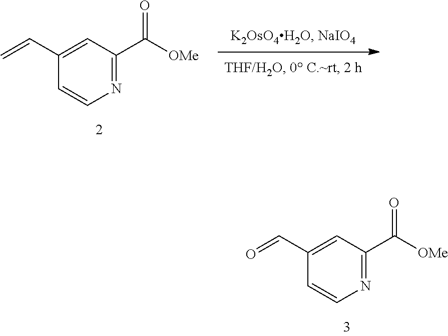

To a stirred solution of methyl 4-vinylpicolinate (3.00 g, 18.38 mmol) and NaIO₄ (11.79 g, 55.13 mmol) in THF (10 mL) H₂O (10 mL) was added K₂OsO₄·2H₂O (0.34 g, 0.92 mmol) in portions at 0° C. under nitrogen atmosphere. The resulting mixture was stirred for 2 h at room temperature under nitrogen atmosphere. The reaction was monitored by LCMS. The resulting mixture was diluted with H₂O (300 mL). The resulting mixture was extracted with EtOAc (3×100 mL). The combined organic layers were washed with brine (3×50 mL), dried over anhydrous Na₂SO₄. After filtration, the filtrate was concentrated under reduced pressure. The residue was purified by silica gel column chromatography, eluted with petroleum ether/EtOAc (2:1 to 1:1) to afford methyl 4-formylpicolinate (2.6 g, 86%) as a yellow solid: ¹H NMR (400 MHz, CDCl₃) δ 10.17 (s, 1H), 9.02 (dd, J=4.9, 0.9 Hz, 1H), 8.53 (dd, J=1.6, 0.9 Hz, 1H), 7.91 (dd, J=4.8, 1.6 Hz, 1H), 4.07 (s, 3H); LC/MS (ESI, m/z): [(M+1)]⁺=166.10.

### Preparation of Methyl 4-(difluoromethyl)picolinate

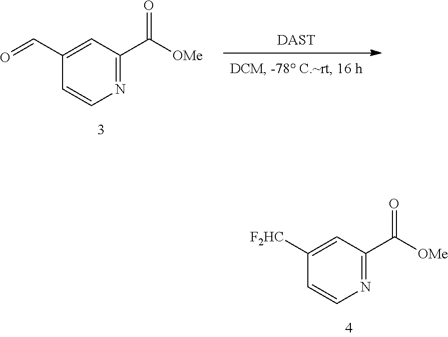 [SMILES: COC(=O)c1cc(C=O)ccn1 ; "DAST" ; COC(=O)c1cc(C(F)F)ccn1]

To a stirred solution of methyl 4-formylpicolinate (2.5 g, 15.13 mmol) in DCM (25 mL) was added DAST (7.3 g, 45.41 mmol) dropwise at −78° C. under nitrogen atmosphere. The resulting mixture was stirred for 16 h at room temperature under nitrogen atmosphere. The reaction was monitored by LCMS. The reaction was quenched by the addition of sat. NaHCO₃ (aq.) (10 mL) at −78° C. The resulting mixture was concentrated under reduced pressure. The residue was purified by silica gel column chromatography, eluted with petroleum ether/EtOAc (5:1 to 2:1) to afford methyl 4-(difluoromethyl)picolinate (1.3 g, 45%) as a yellow oil: ¹H NMR (400 MHz, CDCl₃) δ 8.90 (d, J=4.9 Hz, 1H), 8.26 (s, 1H), 7.66-7.60 (m, 1H), 6.71 (t, J=55.4 Hz, 1H), 4.05 (s, 3H); LC/MS (ESI, m/z): [(M+1)]⁺=188.10.

### Preparation of 4-(Difluoromethyl)picolinic acid

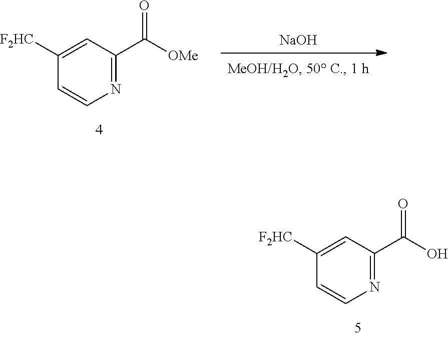 [SMILES: COC(=O)c1cc(C(F)F)ccn1 ; "NaOH" ; O=C(O)c1cc(C(F)F)ccn1]

To a stirred solution of methyl 4-(difluoromethyl)picolinate (1.2 g, 6.41 mmol) and in MeOH (20 mL) and H₂O (2 mL) was added NaOH (512 mg, 12.82 mmol) at room temperature under nitrogen atmosphere. The resulting mixture was stirred for 1 h at 50° C. under nitrogen atmosphere. The reaction was monitored by LCMS. The mixture was neutralized to pH 7 with 1 M HCl (aq.). The resulting mixture was concentrated under reduced pressure to give crude 4-(difluoromethyl)picolinic acid (1.9 g, crude) as a white solid which was used in the next step directly without further purification: ¹H NMR (400 MHz, DMSO-d₆) δ 8.63 (d, J=5.0 Hz, 1H), 8.10 (s, 1H), 7.52 (d, J=5.0 Hz, 1H), 7.13 (t, J=55.3 Hz, 1H); LC/MS (ESI, m/z): [(M+1)]⁺=174.10.

### Preparation of 4-(Difluoromethyl)picolinamide

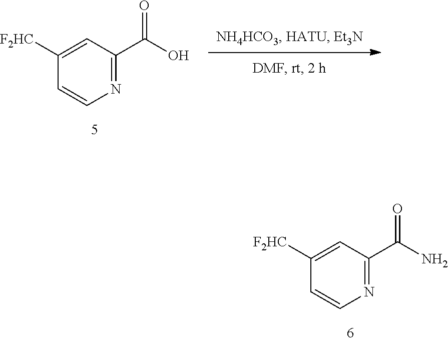

To a stirred solution of 4-(difluoromethyl)picolinic acid (1 g, 5.78 mmol) and NH₄HCO₃ (0.68 g, 8.66 mmol) in DMF (20 mL) were added HATU (3.3 g, 8.66 mmol) and Et₃N (2.9 g, 28.88 mmol) dropwise at room temperature under nitrogen atmosphere. The resulting mixture was stirred for 2 h at room temperature under nitrogen atmosphere. The reaction was monitored by LCMS. The solution was purified by reverse phase Flash chromatography with the following conditions: Column: WelFlash TM C18-I, 20-40 μm, 330 g; Eluent A: Water (plus 10 mM NH₄HCO₃); Eluent B: ACN; Gradient (B %): 5%-25%, 4 min, 25%-45%, 2 min, 45%-95%, 8 min, 85%-95%, 2 min, 95%-95%, 3 min; Flow rate: 80 mL/min; Detector: UV 220 nm; desired fractions were collected at 51% and concentrated under reduced pressure to afford 4-(difluoromethyl)picolinamide (600 mg, 60%) as a white solid: ¹H NMR (400 MHz, DMSO-d₆) δ 8.82 (d, J=5.0 Hz, 1H), 8.19 (d, J=16.1 Hz, 2H), 7.82-7.75 (m, 2H), 7.20 (t, J=55.0 Hz, 1H); LC/MS (ESI, m/z): [(M+1)]⁺=173.10.

### Preparation of 4-(Difluoromethyl)pyridine-2-carbothioamide

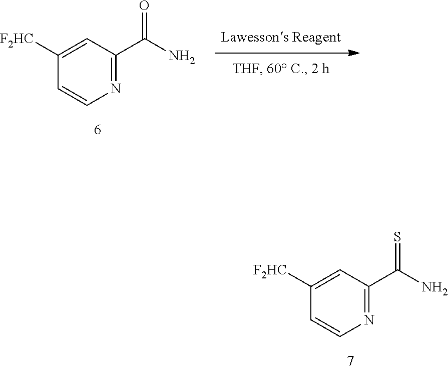

To a stirred solution of 4-(difluoromethyl)picolinamide (300 mg, 1.74 mmol) in THF (6 mL) was added Lawesson reagent (422 mg, 1.04 mmol) at room temperature under nitrogen atmosphere. The resulting mixture was stirred for 2 h at 60° C. under nitrogen atmosphere. The reaction was monitored by LCMS. The solution was purified by reverse phase Flash chromatography with the following conditions: Column: WelFlash TM C18-I, 20-40 μm, 330 g; Eluent A: Water (plus 10 mM NH₄HCO₃); Eluent B: ACN; Gradient (B %): 5%-25%, 4 min, 25%-75%, 2 min, 75%-95%, 8 min, 85%-95%, 2 min, 95%-95%, 3 min; Flow rate: 80 mL/min; Detector: 220/254 nm; desired fractions were collected at 75% and concentrated under reduced pressure to afford 4-(difluoromethyl)pyridine-2-carbothioamide (200 mg, 61%) as a yellow solid: ¹H NMR (400 MHz, DMSO-d₆) δ 10.31 (s, 1H), 10.02 (s, 1H), 8.78 (d, J=4.9 Hz, 1H), 8.65 (d, J=1.6 Hz, 1H), 7.79 (dd, J=5.0, 1.5 Hz, 1H), 7.23 (t, J=55.0 Hz, 1H); LC/MS (ESI, m/z): [(M+1)]⁺=189.05.

### Example 75: (Z)-3-(amino(4-(difluoromethyl)pyridin-2-yl)methylene)-5-bromo indolin-2-one

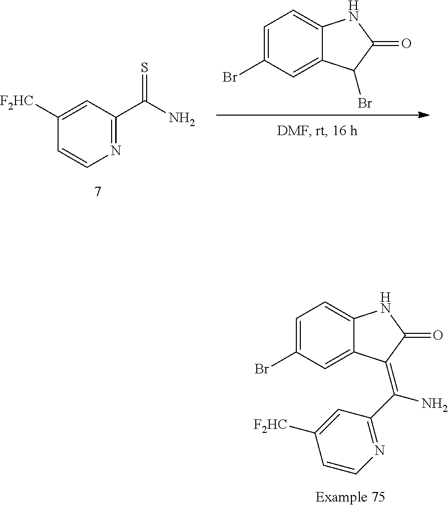 [SMILES: NC(=S)c1cc(C(F)F)ccn1 ; O=C1Nc2ccc(Br)cc2C1Br ; N/C(=C1\C(=O)Nc2ccc(Br)cc21)c1cc(C(F)F)ccn1]

To a stirred solution of 4-(difluoromethyl)pyridine-2-carbothioamide (100 mg, 0.53 mmol) in DMF (4 mL) was added 3,5-dibromo-1,3-dihydroindol-2-one (152 mg, 0.53 mmol) dropwise at room temperature under nitrogen atmosphere. The resulting mixture was stirred for 16 h at room temperature under nitrogen atmosphere. The reaction was monitored by LCMS. The solution was purified by reverse phase Flash chromatography with the following conditions: Column: WelFlash TM C18-I, 20-40 μm, 330 g; Eluent A: Water (plus 10 mM NH₄HCO₃); Eluent B: ACN; Gradient (B %): 5%-25%, 4 min, 25%-75%, 2 min, 55%-95%, 8 min, 85%-95%, 2 min, 95%-95%, 3 min; Flow rate: 80 mL/min; Detector: UV 220 nm; desired fractions were collected at 61% and concentrated under reduced pressure to afford (Z)-3-(amino(4-(difluoro methyl)pyridin-2-yl)methylene)-5-bromoindolin-2-one (36 mg, 18%) as a yellow solid: ¹H NMR (400 MHz, DMSO-d₆) δ 10.59 (s, 1H), 9.50 (d, J=4.3 Hz, 1H), 9.00 (d, J=5.0 Hz, 1H), 8.49 (d, J=4.3 Hz, 1H), 7.88 (d, J=4.8 Hz, 2H), 7.23 (t, J=54.8 Hz, 1H), 7.01 (dd, J=8.2, 2.0 Hz, 1H), 6.76 (d, J=8.2 Hz, 1H), 5.84 (d, J=2.0 Hz, 1H); LC/MS (ESI, m/z): [(M+1)]⁺=366.00.

### Preparation of Enamine Compounds Using Thioamide

### General Procedure

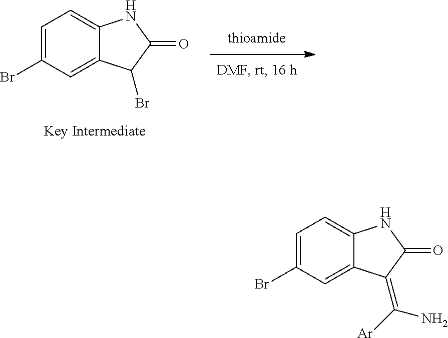

A solution of 3,5-dibromoindolin-2-one (0.2 mmol) and thioamide (0.2 mmol) in DMF (1 mL) was stirred for 16 h at ambient temperature under nitrogen atmosphere. The resulting mixture was purified by reverse phase flash chromatography. The fractions containing desired product were collected and concentrated under reduced pressure to afford the Desired Product.

The following exemplary compounds were prepared using the thioamide-based procedure above.

| Ex. | | | MS: | |
|---|---|---|---|---|
| No. | Structure | Name | [(M + 1)]⁺ | ¹H NMR |
| 76 | 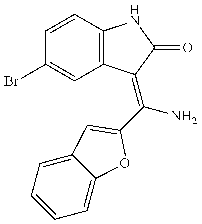 [SMILES: N/C(=C1\C(=O)Nc2ccc(Br)cc21)c1cc2ccccc2o1] | (Z)-3-(amino (benzofuran-2- yl)methylene)-5- bromoindolin-2- one | 355.00 | ¹H NMR (400 MHz, DMSO-d₆) δ 10.66 (s, 1H), 9.55 (s, 1H), 8.37 (s, 1H), 7.87 (dd, J = 7.7, 1.2 Hz, 1H), 7.74 (dd, J = 8.3, 1.0 Hz, 1H), 7.61 (d, J = 1.0 Hz, 1H), 7.56-7.50 (m, 1H), 7.46-7.38 (m, 1H), 7.09 (dd, J = 8.2, 2.0 Hz, 1H), 6.90 (d, J = 2.0 Hz, 1H), 6.81 (d, J = 8.2 Hz, 1H). |
| 77 | 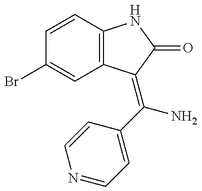 [SMILES: N/C(=C1\C(=O)Nc2ccc(Br)cc21)c1ccncc1] | (Z)-3-(amino (pyridin-4- yl)methylene)-5- bromoindolin-2- one | 316.00 | ¹H NMR (400 MHz, DMSO-d₆) δ 10.60 (s, 1H), 9.44 (d, J = 4.1 Hz, 1H), 8.88- 8.82 (m, 2H), 8.44 (d, J = 4.2 Hz, 1H), 7.60-7.54 (m, 2H), 7.01 (dd, J = 8.2, 2.0 Hz, 1H), 6.76 (d, J = 8.2 Hz, 1H), 5.94 (d, J = 2.0 Hz, 1H). |
| 78 | 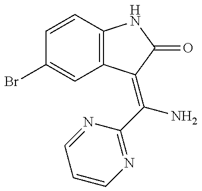 [SMILES: N/C(=C1\C(=O)Nc2ccc(Br)cc21)c1ncccn1] | (Z)-3-(amino (pyrimidin-2- yl)methylene)-5- bromoindolin-2- one | 317.05 | ¹H NMR (400 MHz, DMSO-d₆) δ 10.60 (s, 1H), 9.58 (d, J = 4.1 Hz, 1H), 9.12 (d, J = 4.9 Hz, 2H), 8.47 (d, J = 4.0 Hz, 1H), 7.81 (t, J = 4.9 Hz, 1H), 7.03 (dd, J = 8.2, 2.0 Hz, 1H), 6.76 (d, J = 8.2 Hz, 1H), 6.05 (d, J = 2.0 Hz, 1H). |
| 79 | 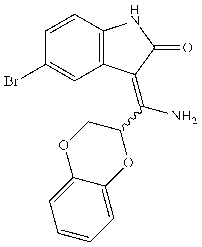 [SMILES: NC(=C1C(=O)Nc2ccc(Br)cc21)C1COc2ccccc2O1] | 3-(Amino(2,3- dihydrobenzo[b] [1,4]dioxin-2- yl)methylene)-5- bromoindolin-2- one | 373.00 | ¹H NMR (400 MHz, DMSO-d₆) δ 10.63 (s, 0.7H), 10.31 (s, 0.3H), 9.73 (s, 0.7H), 8.13 (s, 0.7H), 7.95 (d, J = 1.9 Hz, 0.3H), 7.68 (s, 0.6H), 7.26 (d, J = 1.9 Hz, 0.7H), 7.17-7.10 (m, 1H), 7.09- 6.99 (m, 1.7H), 6.99-6.87 (m, 2.3H), 6.82 (d, J = 8.2 Hz, 0.7H), 6.73 (d, J = 8.2 Hz, 0.3H), 6.38 (dd, J = 8.5, 2.4 Hz, 0.3H), 5.73 (dd, J = 8.9, 2.4 Hz, 0.7H), 4.56-4.41 (m, 1H), 4.09-3.92 (m, 1H). |
| 80 | 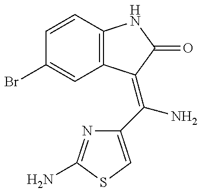 [SMILES: N/C(=C1\C(=O)Nc2ccc(Br)cc21)c1csc(N)n1] | (Z)-3-(amino(2- aminothiazol-4- yl)methylene)-5- bromoindolin-2- one | 337.00 | ¹H NMR (400 MHz, DMSO-d₆) δ 10.43 (s, 1H), 9.47 (d, J = 4.4 Hz, 1H), 8.04 (d, J = 4.5 Hz, 1H), 7.30 (s, 2H), 7.06 (s, 1H), 7.00 (dd, J = 8.1, 2.0 Hz, 1H), 6.93 (d, J = 2.0 Hz, 1H), 6.73 (d, J = 8.2 Hz, 1H). |
| 81 | 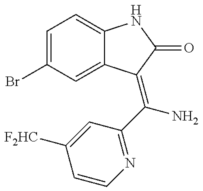 [SMILES: N/C(=C1\C(=O)Nc2ccc(Br)cc21)c1cc(C(F)F)ccn1] | (Z)-3-(amino(4- (difluoromethyl) pyridin-2- yl)methylene)-5- bromoindolin-2- one | 366.00 | ¹H NMR (400 MHz, DMSO-d₆) δ 10.59 (s, 1H), 9.50 (d, J = 4.3 Hz, 1H), 9.00 (d, J = 5.0 Hz, 1H), 8.49 (d, J = 4.3 Hz, 1H), 7.88 (d, J = 4.8 Hz, 2H), 7.23 (t, J = 54.8 Hz, 1H), 7.01 (dd, J = 8.2, 2.0 Hz, 1H), 6.76 (d, J = 8.2 Hz, 1H), 5.84 (d, J = 2.0 Hz, 1H). |

The following intermediate compounds were prepared using the using the thioamide-based procedure above. PGP 227, IRE

| | | MS: | |
|---|---|---|---|
| Structure | Name | [(M + 1)]⁺ | ¹H NMR |
| 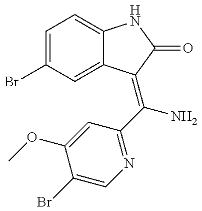 [SMILES: COc1cc(/C(N)=C2/C(=O)Nc3ccc(Br)cc32)ncc1Br] | (Z)-3-(amino(5- bromo-4-methoxy pyridin-2-yl) methylene)-5- bromoindolin-2-one | 424.15 | ¹H NMR (400 MHz, DMSO-d₆) δ 10.56 (s, 1H), 9.53 (d, J = 4.3 Hz, 1H), 8.82 (s, 1H), 8.36 (d, J = 3.8 Hz, 1H), 7.52 (s, 1H), 7.02 (dd, J = 8.2, 2.0 Hz, 1H), 6.77 (d, J = 8.2 Hz, 1H), 6.17 (d, J = 2.0 Hz, 1H), 4.01 (s, 3H). |
| 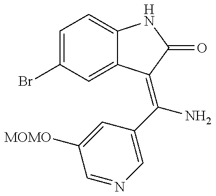 | (Z)-3-(amino(5- (methoxymethoxy) pyridin-3-yl) methylene)-5- bromoindolin-2-one | 376.05 | ¹H NMR (400 MHz, DMSO-d₆) δ 10.60 (s, 1H), 9.50 (d, J = 4.2 Hz, 1H), 8.61 (d, J = 2.8 Hz, 1H), 7.67 (dd, J = 2.8, 1.8 Hz, 1H), 7.01 (dd, J = 8.2, 2.0 Hz, 1H), 6.77 (d, J = 8.2 Hz, 1H), 6.06 (d, J = 2.0 Hz, 1H), 5.36 (s, 2H), 3.43 (s, 3H). |
| 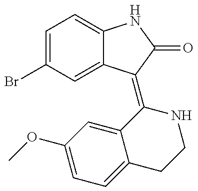 [SMILES: COc1ccc2c(c1)/C(=C1/C(=O)Nc3ccc(Br)cc31)NCC2] | (Z)-5-bromo-3-(7- methoxy-3,4- dihydroisoquinolin- 1(2H)-ylidene) indolin-2-one | 371.05 | ¹H NMR (400 MHz, DMSO-d₆) δ 11.00 (s, 1H), 10.59 (s, 1H), 7.59 (d, J = 2.7 Hz, 1H), 7.44 (d, J = 2.0 Hz, 1H), 7.39 (d, J = 8.4 Hz, 1H), 7.16 (dd, J = 8.4, 2.7 Hz, 1H), 7.03 (dd, J = 8.2, 2.0 Hz, 1H), 6.81 (d, J = 8.2 Hz, 1H), 3.79 (s, 3H), 3.46 (s, 2H), 2.83 (t, J = 6.3 Hz, 2H). |
| 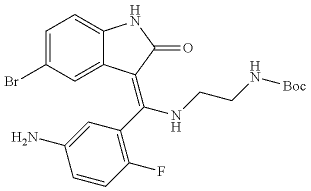 [SMILES: CC(C)(C)OC(=O)NCCN/C(=C1\C(=O)Nc2ccc(Br)cc21)c1cc(N)ccc1F] | Tert-butyl (Z)-(2- (((5-amino-2- fluorophenyl)(5- bromo-2- oxoindolin-3- ylidene)methyl) amino)ethyl) carbamate | 491.10 | ¹H NMR (400 MHz, CDCl₃) δ 10.15 (s, 1H), 7.11 (t, J = 8.7 Hz, 1H), 7.01 (d, J = 8.3 Hz, 1H), 6.97-6.87 (m, 1H), 6.72 (d, J = 8.3 Hz, 1H), 6.66-6.56 (m, 1H), 5.96 (s, 1H), 4.95 (s, 1H), 3.41- 3.15 (m, 4H), 1.42 (s, 9H). |

### Example 82: (Z)-5-bromo-3-(7-hydroxy-3,4-dihydroisoquinolin-1(2H)-ylidene)indolin-2-one

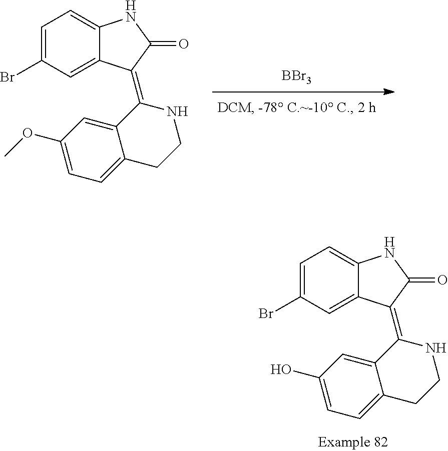 [SMILES: COc1ccc2c(c1)/C(=C1/C(=O)Nc3ccc(Br)cc31)NCC2 ; "BBr3" ; O=C1Nc2ccc(Br)cc2/C1=C1/NCCc2ccc(O)cc21]

To a stirred solution of (Z)-5-bromo-3-(7-methoxy-3,4-dihydroisoquinolin-1(2H)-ylidene)indolin-2-one (100 mg, 0.27 mmol) in DCM (5 mL) was added BBr₃ (2.7 mL, 2.69 mmol, 1M in DCM) dropwise at −78° C. under nitrogen atmosphere. The resulting mixture was stirred for 2 h at −10° C. under nitrogen atmosphere. The reaction was quenched by the addition of sat. NaHCO₃ (aq.) (3 mL) at −78° C. The resulting mixture was concentrated under reduced pressure. The residue was purified by reverse phase Flash chromatography with the following conditions: Column: WelFlash TM C18-I, 20-40 μm, 120 g; Eluent A: Water (plus 0.1% HCOOH); Eluent B: ACN; Gradient: 5%-25% B in 4 min, 25%-55% B in 2 min, 55%-95% B in 8 min, 95%-95% B in 3 min; Flow rate: 60 mL/min; Detector: UV 220/254 nm. The fractions containing desired product were collected at 65% B and concentrated under reduced pressure to afford (Z)-5-bromo-3-(7-hydroxy-3,4-dihydroisoquinolin-1(2H)-ylidene)indolin-2-one (54 mg, 56%) as an orange solid: ¹H NMR (400 MHz, DMSO-d₆) δ 10.96 (s, 1H), 10.55 (s, 1H), 9.72 (s, 1H), 7.45 (d, J=2.5 Hz, 1H), 7.40 (d, J=1.9 Hz, 1H), 7.26 (d, J=8.2 Hz, 1H), 7.00 (dd, J=8.2, 2.2 Hz, 2H), 6.79 (d, J=8.2 Hz, 1H), 3.44 (d, J=6.0 Hz, 2H), 2.77 (t, J=6.2 Hz, 2H); LC/MS (ESI, m/z): [(M+1)]⁺=357.05.

### Example 83: (Z)-3-(amino(5-bromo-4-hydroxypyridin-2-yl)methylene)-5-bromoindolin-2-one

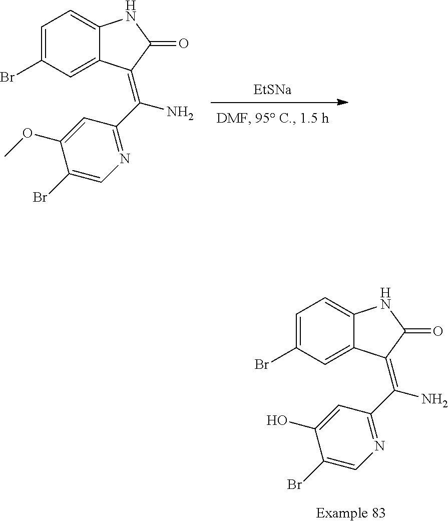 [SMILES: "EtSNa" ; COc1cc(/C(N)=C2/C(=O)Nc3ccc(Br)cc32)ncc1Br ; N/C(=C1\C(=O)Nc2ccc(Br)cc21)c1cc(O)c(Br)cn1]

A solution of (Z)-3-(amino(5-bromo-4-methoxypyridin-2-yl)methylene)-5-bromoindolin-2-one (100 mg, 0.24 mmol) and (ethylsulfanyl)sodium (99 mg, 1.18 mmol) in DMF (1.5 mL) was stirred for 1.5 h at 95° C. under nitrogen atmosphere. After cooling down to ambient temperature, the mixture was neutralized to pH=7 with 2 M HCl (aq.). The resulting mixture was purified by reverse flash chromatography with the following conditions: Column: Spherical C18, 20-40 μm, 120 g; Eluent A: Water (plus 10 mM NH₄HCO₃), Eluent B: ACN; Flow rate: 50 mL/min; Gradient (B %): 5%-22%, 4 min; 22%-40%, 20 min; 40%-95%; 2 min; 95%, 5 min; Detector: UV 254 nm. The fractions containing desired product were collected at 31% B and concentrated under reduced pressure to afford (Z)-3-(amino(5-bromo-4-hydroxypyridin-2-yl)methylene)-5-bromoindolin-2-one (53 mg, 54%) as a yellow solid: ¹H NMR (400 MHz, DMSO-d₆) δ 10.57 (s, 1H), 9.35 (s, 1H), 8.61 (s, 1H), 8.41 (s, 1H), 7.05 (dd, J=8.3, 1.9 Hz, 1H), 6.78 (d, J=8.2 Hz, 1H), 6.20-6.15 (m, 1H); LC/MS (ESI, m/z): [(M+1)]+=409.90.

### Example 84: (Z)-3-(amino(5-hydroxypyridin-3-yl)methylene)-5-bromoindolin-2-one

### Example 84

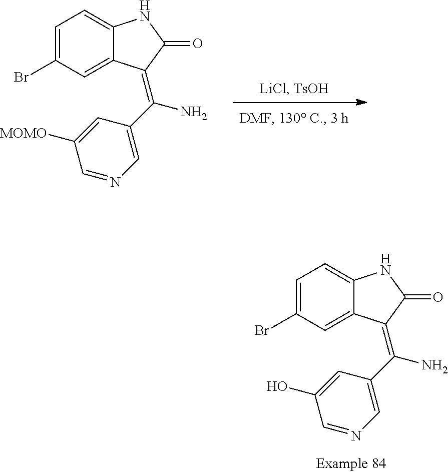

A mixture of (Z)-3-(amino(5-(methoxymethoxy)pyridin-3-yl)methylene)-5-bromo indolin-2-one (100 mg, 0.27 mmol), TsOH (229 mg, 1.33 mmol) and LiCl (57 mg, 1.33 mmol) in DMF (2 mL) was stirred for 3 h at 130° C. The mixture was allowed to cool down to room temperature. The mixture was purified by reverse phase flash chromatography with the following conditions: Column: Spherical C18, 20-40 μm, 330 g; Eluent A: Water (plus 10 mM NH₄HCO₃); Eluent B: ACN; Flow rate: 80 mL/min; Gradient: 5%-5% B, 10 min, 60% B-80% B in 20 min; Detector: UV 220 nm. The fractions containing the desired product were collected at 77% B and concentrated under reduced pressure to afford (Z)-3-(amino(5-hydroxypyridin-3-yl)methylene)-5-bromoindolin-2-one (19 mg, 21%) as a yellow solid: ¹H NMR (400 MHz, DMSO-d₆) δ 10.55 (s, 1H), 10.43 (s, 1H), 9.46 (s, 1H), 8.38 (d, J=2.8 Hz, 2H), 8.16 (d, J=1.8 Hz, 1H), 7.28 (t, J=2.3 Hz, 1H), 7.01 (dd, J=8.2, 2.0 Hz, 1H), 6.76 (d, J=8.2 Hz, 1H), 6.06 (d, J=2.0 Hz, 1H); LC/MS (ESI, m/z): [(M+1)]⁺=332.05.

### Example 85: (E)-5-bromo-3-(1-(3-hydroxyphenyl)ethylidene)indolin-2-one

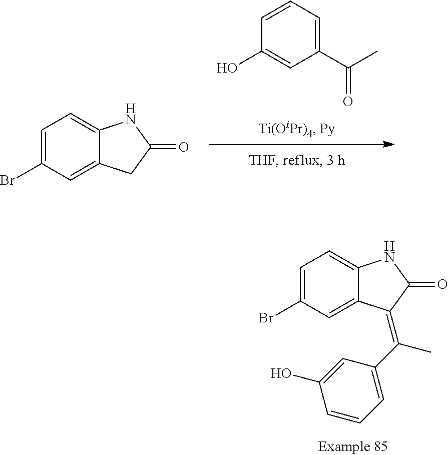

To a stirred solution of 5-bromo-1,3-dihydroindol-2-one (300 mg, 1.41 mmol) and 3-hydroxyacetophenone (174 mg, 1.27 mmol) in THF (6 mL) were added Ti(Oi-Pr)₄ (1.21 g, 4.24 mmol) and pyridine (224 mg, 2.83 mmol) dropwise at room temperature under nitrogen atmosphere. The resulting mixture was stirred for 3 h at 60° C. under nitrogen atmosphere. The reaction was monitored by LCMS. The resulting mixture was diluted with water (10 mL). The resulting mixture was filtered, the filter cake was washed with EtOAc/THF (1/5) (3×10 mL). The filtrate was concentrated under reduced pressure. The residue was purified by reverse phase flash chromatography with the following conditions: Column: WelFlash TM C18-I, 20-40 μm, 120 g; Eluent A: Water (plus 0.1% HCOOH); Eluent B: ACN; Gradient: 5%-40% B in 6 min; 40%-65% B in 10 min; 65%-95% B in 2 min; Flow rate: 50 mL/min; Detector: 220/254 nm. Desired fractions were collected at 55% B and concentrated under reduced pressure to afford (E)-5-bromo-3-(1-(3-hydroxyphenyl) ethylidene)indolin-2-one (122 mg, 27%) as a yellow solid: ¹H NMR (400 MHz, DMSO-d₆) δ 10.72 (s, 1H), 9.74 (s, 1H), 7.35 (t, J=7.8 Hz, 1H), 7.24 (dd, J=8.3, 2.0 Hz, 1H), 6.90 (dd, J=8.2, 1.6 Hz, 1H), 6.75-6.70 (m, 2H), 6.68 (t, J=2.0 Hz, 1H), 6.11 (d, J=2.0 Hz, 1H), 2.68 (s, 3H); LC/MS (ESI, m/z): [(M+1)]⁺=329.95.

The following exemplary compounds were prepared using the procedure of Example 85 with the relevant ketones.

| Ex. | | | MS: | |
|---|---|---|---|---|
| No. | Structure | Name | [(M + 1)]⁺ | ¹H NMR |
| 86 | 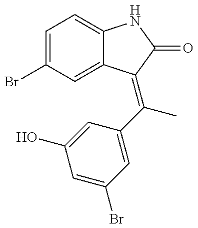 [SMILES: C/C(=C1\C(=O)Nc2ccc(Br)cc21)c1cc(O)cc(Br)c1] | (E)-5-bromo-3-(1-(3- bromo-5-hydroxy phenyl)ethylidene) indolin-2-one | 409.85 | ¹H NMR (400 MHz, DMSO-d₆) δ 10.72 (s, 1H), 10.25 (s, 1H), 7.27 (dd, J = 8.3, 2.0 Hz, 1H), 7.09 (t, J = 2.0 Hz, 1H), 6.97 (t, J = 1.6 Hz, 1H), 6.75 (d, J = 8.3 Hz, 1H), 6.71 (t, J = 1.8 Hz, 1H), 6.16 (d, J = 2.0 Hz, 1H), 2.66 (s, 3H). |
| 87 | 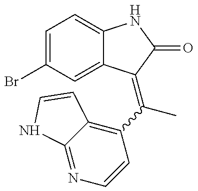 [SMILES: CC(=C1C(=O)Nc2ccc(Br)cc21)c1ccnc2[nH]ccc12] | 3-(1-(1H-pyrrolo[2,3- b]pyridin-4-yl) ethylidene)-5- bromoindolin-2-one | 353.95 | ¹H NMR (400 MHz, DMSO-d₆) δ 11.89 (s, 0.9H), 11.65 (s, 0.1H), 10.78 (s, 0.9H), 10.48 (s, 0.1H), 8.37 (d, J = 4.9 Hz, 0.9H), 8.19 (d, J = 4.9 Hz, 0.1H), 7.82 (s, 0.1H), 7.53 (t, J = 3.0 Hz, 0.9H), 7.47-7.41 (m, 0.2H), 7.20 (dd, J = 8.2, 2.1 Hz, 0.9H), 7.04 (d, J = 4.9 Hz, 0.9H), 6.90 (d, J = 4.8 Hz, 0.1H), 6.81 (d, J = 8.3 Hz, 0.1H), 6.74 (d, J = 8.3 Hz, 0.9H), 6.25 (d, J = 3.4 Hz, 1H), 5.77 (d, J = |
| | | | | 2.0 Hz, 0.9H), 2.79 (s, 2.7H), |
| | | | | 2.62 (s, 0.3H). |
| 88 | 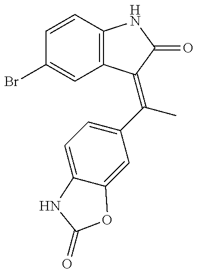 [SMILES: C/C(=C1\C(=O)Nc2ccc(Br)cc21)c1ccc2[nH]c(=O)oc2c1] | (E)-6-(1-(5-bromo-2- oxoindolin-3-ylidene) ethyl)benzo[d]oxazol- 2(3H)-one | 371.10. | ¹H NMR (400 MHz, DMSO-d₆) δ 11.89 (s, 1H), 10.73 (s, 1H), 7.41 (s, 1H), 7.29-7.21 (m, 2H), 7.15 (d, J = 8.0 Hz, 1H), 6.74 (d, J = 8.3 Hz, 1H), 6.13 (s, 1H), 2.71 (s, 3H). |
| 89 | 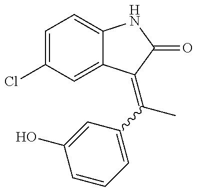 [SMILES: CC(=C1C(=O)Nc2ccc(Cl)cc21)c1cccc(O)c1] | 5-Chloro-3-(1-(3- hydroxyphenyl) ethylidene)indolin- 2-one | 286.00. | ¹H NMR (400 MHz, DMSO-d₆) δ 10.70 (s, 0.82H), 10.45 (s, 0.18H), 9.72 (s, 0.82H), 9.37 (s, 0.18H), 7.61 (d, J = 2.1 Hz, 0.18H), 7.35 (t, J = 7.8 Hz, 0.82H), 7.27 (dd, J = 8.2, 2.1 Hz, 0.18H), 7.16 (d, J = 7.8 Hz, 0.18H), 7.12 (dd, J = 8.4, 2.3 Hz, 0.82H), 6.90 (dd, J = 8.1, 2.4 Hz, 0.82H), 6.82 (d, J = 8.3 Hz, 0.18H), 6.78 (d, J = 8.3 Hz, 0.82H), 6.73 (d, J = 7.8 Hz, |
| | | | | 1.18H), 6.69 (t, J = 2.2 Hz, 1H), |
| | | | | 5.98 (d, J = 2.1 Hz, 0.82H), 2.68 |
| | | | | (s, 2.5H), 2.53 (s, 0.5H). |
| 90 | 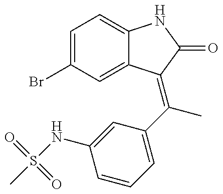 [SMILES: C/C(=C1\C(=O)Nc2ccc(Br)cc21)c1cccc(NS(C)(=O)=O)c1] | (E)-N-(3-(1-(5- bromo-2-oxoindolin- 3-ylidene)ethyl) pheny)methane- sulfonamide | 407.00 | ¹H NMR (400 MHz, DMSO-d₆) δ 10.74 (s, 1H), 9.93 (s, 1H), 7.55 (t, J = 7.9 Hz, 1H), 7.40-7.34 (m, 1H), 7.25 (dd, J = 8.3, 2.0 Hz, 1H), 7.16-7.04 (m, 2H), 6.75 (d, J = 8.3 Hz, 1H), 5.99 (d, J = 2.0 Hz, 1H), 3.01 (s, 3H), 2.70 (s, 3H). |
| 91 | 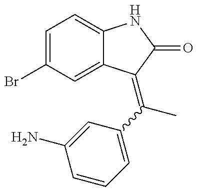 [SMILES: CC(=C1C(=O)Nc2ccc(Br)cc21)c1cccc(N)c1] | 3-(1-(3-Amino phenyl)ethylidene)-5- bromoindolin-2-one | 329.00 | ¹H NMR (400 MHz, DMSO-d₆) δ 10.67 (s, 0.7H), 10.42 (s, 0.3H), 7.70 (d, J = 1.9 Hz, 0.3H), 7.38 (dd, J = 8.2, 2.0 Hz, 0.3H), 7.22 (dd, J = 8.2, 2.1 Hz, 0.7H), 7.17 (t, J = 7.8 Hz, 0.7H), 6.99 (t, J = 7.7 Hz, 0.3H), 6.77 (d, J = 8.3 Hz, 0.3H), 6.72 (d, J = 8.3 Hz, 0.7H), 6.70-6.62 (m, 0.7H), 6.55-6.49 (m, 0.3H), 6.48-6.37 (m, 2H), 6.26 (d, J = 2.0 Hz, 0.7H), 5.30 (s, 1.4H), 5.03 (s, 0.6H), 2.66 (s, |
| | | | | 2H), 2.49 (1H). |
| 92 | 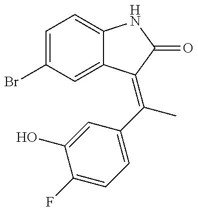 [SMILES: C/C(=C1\C(=O)Nc2ccc(Br)cc21)c1ccc(F)c(O)c1] | (E)-5-bromo-3-(1-(4- fluoro-3-hydroxy phenyl)ethylidene) indolin-2-one | 348.00, 350.00. | ¹H NMR (400 MHz, DMSO-d₆) δ 10.71 (s, 1H), 10.18 (s, 1H), 7.32 (dd, J = 11.3, 8.3 Hz, 1H), 7.25 (dd, J = 8.3, 2.0 Hz, 1H), 6.90 (dd, J = 8.5, 2.2 Hz, 1H), 6.79- 6.73 (m, 2H), 6.15 (d, J = 2.0 Hz, 1H), 2.67 (s, 3H). |
| 93 | 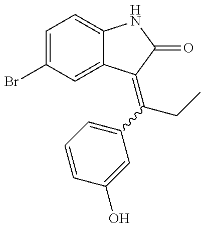 [SMILES: CCC(=C1C(=O)Nc2ccc(Br)cc21)c1cccc(O)c1] | (Z)-5-bromo-3-(1-(3- hydroxyphenyl) propylidene)indolin- 2-one | 344.90 | ¹H NMR (400 MHz, DMSO-d₆) δ 10.45 (s, 1H), 9.33 (s, 1H), 7.63 (d, J = 1.9 Hz, 1H), 7.40 (dd, J = 8.3, 1.9 Hz, 1H), 7.15 (t, J = 7.8 Hz, 1H), 6.78 (d, J = 8.3 Hz, 1H), 6.75-6.69 (m, 1H), 6.63 (dt, J = 7.6, 1.3 Hz, 1H), 6.60 (t, J = 2.0 Hz, 1H), 2.82 (q, J = 7.6 Hz, 2H), 1.07 (t, J = 7.5 Hz, 3H). |
| 94 | 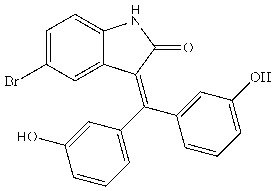 [SMILES: O=C1Nc2ccc(Br)cc2C1=C(c1cccc(O)c1)c1cccc(O)c1] | 3-(Bis(3-hydroxy phenyl)methylene)-5- bromindolin-2-one | 408.10 | ¹H NMR (400 MHz, DMSO-d₆) δ 10.58 (s, 1H), 9.68 (s, 1H), 9.38 (s, 1H), 7.33 (t, J = 7.9 Hz, 1H), 7.26 (dd, J = 8.3, 2.0 Hz, 1H), 7.13 (t, J = 7.9 Hz, 1H), 6.90 (dd, J = 8.6, 2.5 Hz, 1H), 6.77-6.70 (m, 4H), 6.66 (t, J = 2.1 Hz, 1H), 6.61 (t, J = 2.0 Hz, 1H), 6.23 (d, J = 2.0 Hz, 1H). |
| 95 | 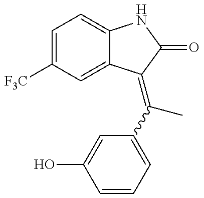 [SMILES: CC(=C1C(=O)Nc2ccc(C(F)(F)F)cc21)c1cccc(O)c1] | 3-(1-(3- Hydroxyphenyl) ethylidene)-5- (trifluoromethyl) indolin-2-one | 320.10 | ¹H NMR (400 MHz, DMSO-d₆) δ 10.95 (s, 0.7H), 10.72 (s, 0.3H), 9.69 (s, 0.7H), 9.37 (s, 0.3H), 7.83 (s, 0.3H), 7.59 (d, J = 8.0 Hz, 0.3H), 7.46-7.39 (m, 0.7H), 7.34 (t, J = 7.8 Hz, 0.7H), 7.16 (t, J = 7.8 Hz, 0.3H), 7.02-6.86 (m, 1.7H), 6.78-6.67 (m, 2.3H), 6.29 (d, J = 1.8 Hz, 0.7H), 2.70 (s, 2H), 2.58 (s, 1H). |
| 96 | 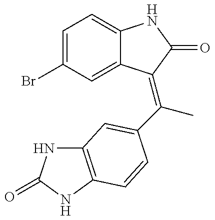 [SMILES: C/C(=C1\C(=O)Nc2ccc(Br)cc21)c1ccc2[nH]c(=O)[nH]c2c1] | (E)-5-(1-(5-bromo-2- oxoindolin-3- ylidene)ethyl)-1,3- dihydro-2H- benzo[d]imidazol-2- one | 370.05 | ¹H NMR (400 MHz, DMSO-d₆) δ 10.83 (s, 1H), 10.79 (s, 1H), 10.67 (s, 1H), 7.22 (dd, J = 8.3, 2.0 Hz, 1H), 7.08 (d, J = 7.9 Hz, 1H), 6.95-6.88 (m, 2H), 6.74 (d, J = 8.3 Hz, 1H), 6.18 (d, J = 2.0 Hz, 1H), 2.71 (s, 3H). |
| 97 | 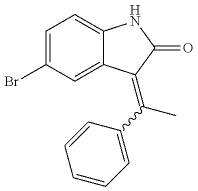 [SMILES: CC(=C1C(=O)Nc2ccc(Br)cc21)c1ccccc1] | 5-Bromo-3-(1- phenylethylidene) indolin-2-one | 314.05 | ¹H NMR (400 MHz, DMSO-d₆) δ 10.71 (s, 1H), 7.61-7.48 (m, 3H), 7.37-7.33 (m, 2H), 7.23 (dd, J = 8.3, 2.0 Hz, 1H), 6.81-6.72 (m, 1H), 5.95 (d, J = 2.0 Hz, 1H), 2.72 (s, 3H). |

### Example 98: 5-Bromo-3-(1-(3-bromo-4,5-dihydroxyphenyl)ethylidene)indolin-2-one

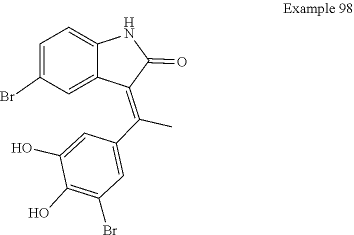 [SMILES: C/C(=C1\C(=O)Nc2ccc(Br)cc21)c1cc(O)c(O)c(Br)c1]

### Preparation of (E)-5-bromo-3-(1-(3-bromo-4,5-bis(methoxymethoxy)phenyl)ethylidene) indolin-2-one

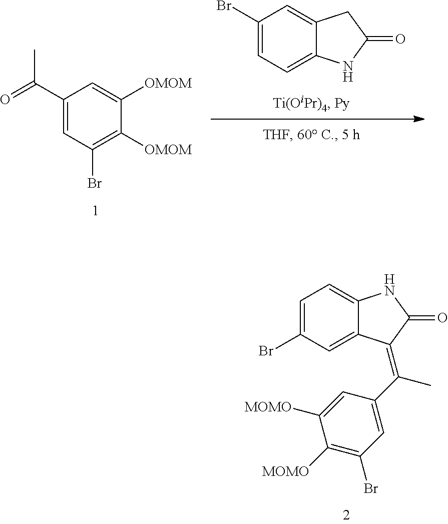

To a stirred solution of 1-(3-bromo-4,5-bis(methoxymethoxy)phenyl)ethanone (300 mg, 0.94 mmol) and 5-bromoindolin-2-one (179 mg, 0.85 mmol) in THF (5 mL) were added Ti(Oi-Pr)₄ (802 mg, 2.82 mmol) and pyridine (149 mg, 1.88 mmol) dropwise at room temperature under nitrogen atmosphere. The resulting mixture was stirred for 5 h at 60° C. under nitrogen atmosphere. The resulting mixture was diluted with water (20 mL). The resulting mixture was filtered, the filter cake was washed with EtOAc/THF (1/5) (3×40 mL). The resulting mixture was extracted with EtOAc (3×30 mL). The combined organic layers were washed with brine (2×30 mL), dried over anhydrous Na₂SO₄. After filtration, the filtrate was concentrated under reduced pressure. The residue was purified by silica gel column chromatography, eluted with petroleum ether/EtOAc (10:1 to 3:1) to afford (E)-5-bromo-3-(1-(3-bromo-4,5-bis(methoxymethoxy)phenyl)ethylidene)indolin-2-one (400 mg, 92%) as a brown oil: ¹H NMR (400 MHz, DMSO-d₆) δ 10.75 (s, 1H), 7.31 (d, J=1.9 Hz, 1H), 7.28 (dd, J=8.3, 2.0 Hz, 1H), 7.17 (d, J=1.9 Hz, 1H), 6.76 (d, J=8.3 Hz, 1H), 6.19 (d, J=2.0 Hz, 1H), 5.24 (s, 4H), 3.59 (s, 3H), 3.40 (s, 3H), 2.70 (s, 3H); LC/MS (ESI, m/z): [(M+1)]+=514.05.

### Preparation of (E)-5-bromo-3-(1-(3-bromo-4,5-dihydroxyphenyl)ethylidene)indolin-2-one

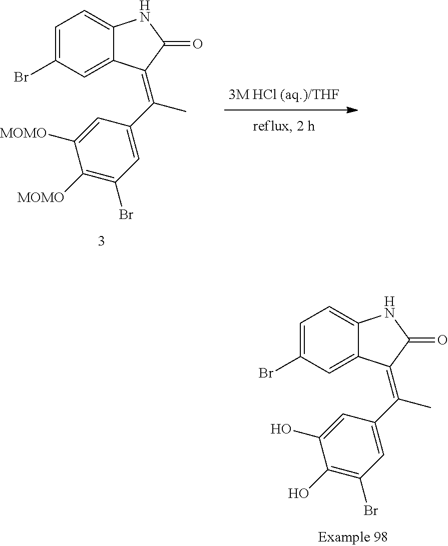

A mixture of (E)-5-bromo-3-(1-(3-bromo-4,5-bis(methoxymethoxy)phenyl)ethylidene) indolin-2-one (200 mg, 0.39 mmol) in THF (3 mL) and 3 M HCl (aq.) (1 mL) was refluxed for 2 h under nitrogen atmosphere. The resulting mixture was concentrated under reduced pressure. The residue was purified by reverse phase flash chromatography with the following conditions: Column: WelFlash TM C18-I, 20-40 μm, 120 g; Eluent A: Water (plus 0.1% HCOOH); Eluent B: ACN; Gradient: 5%-40% B in 5 min; 40%-60% B in 10 min; 60%-95% B in 5 min; Flow rate: 55 mL/min; Detector: UV 220/254 nm. Desired fractions were collected at 54% B and concentrated under reduced pressure to afford (E)-5-bromo-3-(1-(3-bromo-4,5-dihydroxy phenyl)ethylidene)indolin-2-one (18 mg, 11%) as a dark yellow solid: H NMR (400 MHz, DMSO-d₆) δ 10.69 (s, 1H), 10.13 (s, 1H), 9.58 (s, 1H), 7.26 (dd, J=8.2, 2.1 Hz, 1H), 6.97 (d, J=2.0 Hz, 1H), 6.76-6.73 (m, 2H), 6.41 (d, J=2.0 Hz, 1H), 2.65 (s, 3H); LC/MS (ESI, m/z): [(M+1)]⁺=424.95.

The following exemplary compounds were prepared using the procedure of Example 98.

| Ex. | | | MS: | |
|---|---|---|---|---|
| No. | Structure | Name | [(M + 1)]⁺ | ¹H NMR |
| 99 | 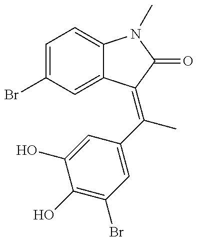 [SMILES: C/C(=C1\C(=O)N(C)c2ccc(Br)cc21)c1cc(O)c(O)c(Br)c1] | (E)-5-bromo-3-(1- (3-bromo-4,5- dihydroxyphenyl) ethylidene)-1- methylindolin-2-one | 439.85 | ¹H NMR (400 MHz, DMSO-d₆) δ 9.86 (br, 2H), 7.37 (dd, J = 8.3, 2.1 Hz, 1H), 6.98-6.92 (m, 2H), 6.74 (d, J = 2.1 Hz, 1H), 6.45 (d, J = 2.0 Hz, 1H), 3.18 (s, 3H), 2.68 (s, 3H). |
| 100 | 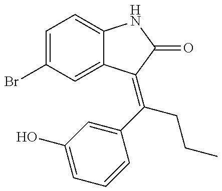 [SMILES: CCC/C(=C1\C(=O)Nc2ccc(Br)cc21)c1cccc(O)c1] | (E)-5-bromo-3-(1- (3- hydroxyphenyl) butylidene)indolin- 2-one | 358.00 | ¹H NMR (400 MHz, DMSO-d₆) δ 10.67 (s, 1H), 9.72 (s, 1H), 7.35 (t, J = 7.8 Hz, 1H), 7.23 (dd, J = 8.3, 2.1 Hz, 1H), 6.93-6.88 (m, 1H), 6.75-6.63 (m, 3H), 6.03 (d, J = 2.0 Hz, 1H), 3.29-3.11 (m, 2H), 1.48-1.36 (m, 2H), 0.92 (t, J = 7.3 Hz, 3H). |
| 101 | 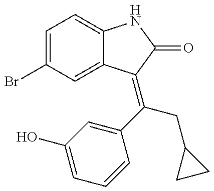 [SMILES: O=C1Nc2ccc(Br)cc2/C1=C(/CC1CC1)c1cccc(O)c1] | (E)-5-bromo-3-(2- cyclopropyl-1-(3- hydroxyphenyl) ethylidene)indolin- 2-one | 369.95 | ¹H NMR (400 MHz, DMSO-d₆) δ 10.71 (s, 1H), 9.74 (s, 1H), 7.36 (t, J = 7.8 Hz, 1H), 7.24 (dd, J = 8.3, 2.1 Hz, 1H), 6.93-6.89 (m, 1H), 6.76-6.61 (m, 3H), 6.01 (d, J = 2.0 Hz, 1H), 5.89-5.76 (m, 1H), 5.07-4.93 (m, 2H), 3.41-3.25 (m, 2H), 2.19-2.09 (m, 2H). |

### Example 102: (E)-5-bromo-3-(1-(3-hydroxyphenyl)-2-phenylethylidene)indolin-2-one

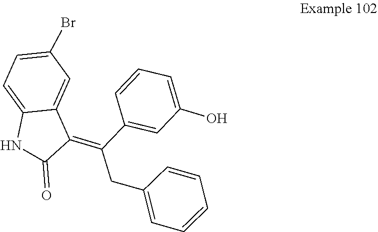 [SMILES: O=C1Nc2ccc(Br)cc2/C1=C(/Cc1ccccc1)c1cccc(O)c1]

### Preparation of (E)-5-Bromo-3-(1-(3-hydroxphenyl)₂-phenylethylidene)indolin-2-one

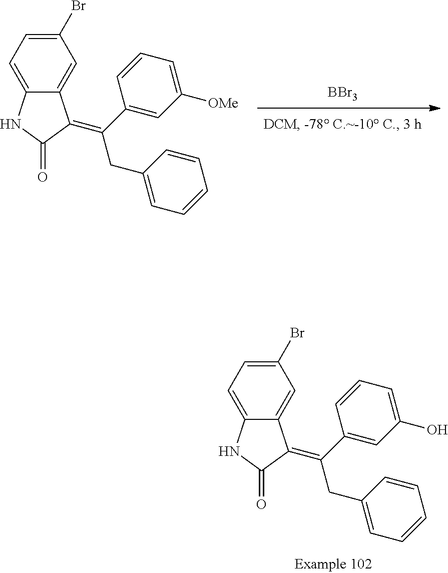 [SMILES: COc1cccc(/C(Cc2ccccc2)=C2/C(=O)Nc3ccc(Br)cc32)c1 ; "BBr3" ; O=C1Nc2ccc(Br)cc2/C1=C(/Cc1ccccc1)c1cccc(O)c1]

To a stirred solution of (E)-5-bromo-3-(1-(3-methoxyphenyl)-2-phenylethylidene)indolin-2-one (280 mg, 0.67 mmol) in DCM (4 mL) was added BBr₃ (6.7 mL, 6.66 mmol, 1 M in DCM) dropwise at −78° C. under nitrogen atmosphere. The resulting mixture was stirred for 3 h at −10° C. under nitrogen atmosphere. The reaction was quenched by the addition of sat. NaHCO₃ (aq.) (40 mL) at −78° C. Then MeOH (5 mL) was added to the mixture. The resulting mixture was filtered, the filter cake was washed with water (3×50 mL) and EtOAc (2×20 mL). The resulting solid was dried under infrared light. This resulted in (E)-5-bromo-3-(1-(3-hydroxyphenyl)-2-phenylethylidene)indolin-2-one (137 mg, 52%) as a yellow solid: ¹H NMR (400 MHz, DMSO-d₆) δ 10.81 (s, 1H), 9.57 (s, 1H), 7.29-7.18 (m, 4H), 7.18-7.14 (m, 1H), 7.14-7.08 (m, 2H), 6.85-6.79 (m, 1H), 6.76 (d, J=8.3 Hz, 1H), 6.50-6.41 (m, 2H), 6.01 (d, J=2.1 Hz, 1H), 4.64 (s, 2H); LC/MS (ESI, m/z): [(M+1)]⁺=406.10. Chemical Formula: C₂₂H₁₆BrNO₂, Exact Mass: 405.04.

### Protocol for MSD Assay: Daoy-ATXN1(600)-mATXN1 MSD (CTG normalized)

### Generation of Daoy-Flag-ATXN1-60Q stable line

The Flag-ATXN1(60Q) gene is synthesized by Genescript and cloned into the pLVX-IRES-Puro vector (Takarabio, catalog No. 632183) using EcoRI/BamHI restriction enzyme sites. Lentivirus is made by transfecting HEK293T cells with pLVX-IRES-Puro-Flag-ATXN1(60Q) plasmids and packaging plasmids psPAX2 and pMD2.G at 4:3:1 ratio (8:6:2 g, respectively) in 10 cm plates, and are harvested at 48 hours post transfection. Daoy cells (ATCC catalog No. HTB-186) seeded in T25 flasks are infected with the lentivirus in the presence of 8 g/mL polybrene (Sigma, TR-1003-G) for 24 hours. 48 hours post infection, the cells are selected using 0.5 g/mL puromycin (Sigma, P8833) for more than 5 days until all cells in the uninfected group die. The expression of Flag-ATXN1(60Q) is verified by anti-Flag immunoblot using anti-Flag antibody (Sigma, F1804-200UG, M2).

### Sample Preparation

As explained above, Daoy-Flag-ATXN1(60Q) cells are prepared as a stable cell line by infecting Daoy cells with lentivirus. The media for the cells is EMEM (ATTC, 30-2008) with 10% fetal bovin serum (FBS).

On Day 1, the Daoy-Flag-ATXN1(60Q) cells are seeded in a 96 well plate at a concentration of 1.2×10⁴/well. On Day 2, the cells are treated with test compounds at test concentrations (compounds are serially diluted with DMSO to obtain test concentrations, such that final DMSO concentration is 0.3%). On Day 3, the cells are washed with 100 μL of phosphate-buffered saline (PBS) per well, and then 30 μL of radioimmuno-precipitation assay (RIPA) buffer (with protease and phosphatase inhibitor cocktail) is added to each well.

The well plate is then cooled on ice for 30 minutes in order to complete lysis, and MSD samples are prepared using 10 μL of Daoy-Flag-ATXN1-60Q lysate and 90 μL of RIPA buffer with gentle mixing. Each MSD sample has a total volume of 100 μL, and is used immediately or stored at −80° C.

### MSD Protocol

A standard Meso Scale Discovery (MSD) plate is coated with anti-Flag (Sigma, F1804-200UG, M2) in PBS at 1:500 dilution ratio and 40 L/well, and is incubated at 4° C. overnight with shaking at 600 rpm. Then, the MSD plate is washed 3 times with 150 L/well of 1X TBST (tris-buffered saline (TBS) with Tween 20) buffer, and the MSD plate is blocked with 150 μL of blocking buffer [3% Blocker A in 1X TBST, (3 g Blocker A per 100 mL 1X TBST)] and shaken for 1 hour at 600 rpm at room temperature. Then, the MSD plate is washed three times with 150 μL/well of 1X TBST. Next, the diluted lysate samples (from Sample Preparation above) are added to the MSD plate at 25 μL/well, and the MSD plate is shaken at 600 rpm for 1 hour at room temperature. Then the MSD plate is washed 3 times with 150 μL/well of 1X TBST.

Detection antibody anti-Ataxin 1 (G-bio, ITA1916) (1:500), which is prepared in antibody detection buffer (1% Blocker A in 1X TBST), is then added to the MSD plate at 25 L/well, and the MSD plate is shaken at 600 rpm for 1 hour at room temperature. The MSD plate is then washed three times with 150 μL/well of 1X TBST. Next, a secondary detection antibody, Goat anti-rabit (GAR) with SULFO-TAG (1:500), which is prepared in antibody detection buffer (1% Blocker A in 1X TBST), is added at 25 μL/well and the MSD plate is shaken at 600 rpm for 1 hour at room temperature. Then the MSD plate is washed 3 times with 150 μL/well of 1X TBST, and 2X MSD reading buffer is added at 150 μL/well (diluted from 4x MSD reading buffer with water). Finally, the signal from each well is measured on the MSD Meso QuickPle SQ120 Plate Reader. DC₅₀ data is calculated using collaborate drug discovery (CDD) software (https://www.collaborativedrug.com/about).

The results obtained in these experiments for compounds prepared according to the examples are summarized in Table 1 below. Table 1 shows the activity of the Examples 1-102 in the Daoy-ATXN1(60Q)-mATXN1 MSD assay (CTG normalized), wherein “A” represents DC₅₀ of less than 10.0 μM, “B” represents DC50 of equal to or greater than 10.0 μM and equal to or less than 30.0 μM (10.0 μM<DC50≤30.0 μM), and “C” represents DC₅₀ of greater than 30.0 μM.

TABLE 1

| | Daoy-ATXN1(60Q)-mATXN1 MSD |
|---|---|
| Example | (CTG normalized) |
| No. | DC₅₀ (μM) |
| 1 | A |
| 2 | N/A |
| 3 | N/A |
| 4 | N/A |
| 5 | A |
| 6 | A |
| 7 | A |
| 8 | A |
| 9 | A |
| 10 | N/A |
| 11 | N/A |
| 12 | C |
| 13 | N/A |
| 14 | N/A |
| 15 | A |
| 16 | N/A |
| 17 | A |
| 18 | A |
| 19 | A |
| 20 | A |
| 21 | C |
| 22 | B |
| 23 | N/A |
| 24 | C |
| 25 | A |
| 26 | A |
| 27 | A |
| 28 | B |
| 29 | N/A |
| 30 | A |
| 31 | N/A |
| 32 | A |
| 33 | A |
| 34 | A |
| 35 | A |
| 36 | B |
| 37 | A |
| 38 | A |
| 39 | A |
| 40 | A |
| 41 | A |
| 42 | C |
| 43 | C |
| 44 | C |
| 45 | A |
| 46 | C |
| 47 | C |
| 48 | C |
| 49 | C |
| 50 | B |
| 51 | C |
| 52 | C |
| 53 | B |
| 54 | C |
| 55 | C |
| 56 | A |
| 57 | B |
| 58 | A |
| 59 | A |
| 60 | A |
| 61 | B |
| 62 | C |
| 63 | A |
| 64 | A |
| 65 | N/A |
| 66 | A |
| 67 | N/A |
| 68 | A |
| 69 | A |
| 70 | A |
| 71 | A |
| 72 | A |
| 73 | A |
| 74 | B |
| 75 | B |
| 76 | A |
| 77 | A |
| 78 | C |
| 79 | C |
| 80 | N/A |
| 81 | B |
| 82 | N/A |
| 83 | C |
| 84 | A |
| 85 | B |
| 86 | N/A |
| 87 | N/A |
| 88 | C |
| 89 | N/A |
| 90 | N/A |
| 91 | N/A |
| 92 | A |
| 93 | N/A |
| 94 | N/A |
| 95 | A |
| 96 | N/A |
| 97 | N/A |
| 98 | C |
| 99 | N/A |
| 100 | A |
| 101 | N/A |
| 102 | N/A |

“N/A”-means not tested

### Equivalents

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

Those skilled in the art will recognize or be able to ascertain using no more than routine experimentation, many equivalents to the specific embodiments of the disclosure described and claimed herein. Such equivalents are intended to be encompassed by the following claims.

What is claimed is:

1. A compound of Formula (I): 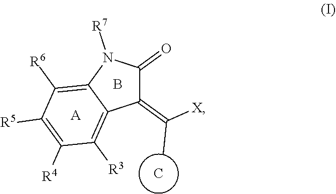
or a pharmaceutically acceptable salt thereof, wherein:
X is selected from NH₂, —N(R^{N})—R¹, ring D and —N(R^{N})-ring D and C₁₋₆ alkyl optionally substituted with one or more R¹⁰;
each R^{N} is independently selected from H, C₁₋₆ alkyl and C₃₋₇ cycloaliphatic;
R¹ is C₁₋₆ alkyl optionally substituted with one or more R¹⁰;
each R¹⁰ is independently selected from halo, —CN, —OR¹⁵, —SR¹⁵, —S(O)R¹⁰⁰, —SO₂R¹⁰⁰, —S(O)N(R¹⁰⁰)₂, —SO₂N(R¹⁰⁰)₂, —OC(O)R¹⁰⁰, —OC(O)N(R¹⁰⁰)₂, —N(R¹⁰⁰)₂, —N(R¹⁰⁰)C(O)R¹⁰⁰, —N(R¹⁰⁰)CO₂R¹⁰⁰, —N(R¹⁰⁰)C(O)N(R¹⁰⁰)₂, —N(R¹⁰⁰)SO₂R¹⁰⁰, C(O)R¹⁰⁰, —CO₂R¹⁰⁰, —C(O)N(R¹⁰⁰)₂, —C(O)N(R¹⁰⁰)OR¹⁵ and phenyl;
each R¹⁵ is independently selected from H, C₁₋₆ alkyl and —(CH₂CH₂O)ₙ—R¹⁶,
wherein n is 1, 2, 3, 4 or 5;
each R¹⁶ is independently selected from H, C₁₋₆ alkyl and —N(R^{N})C(O)R¹⁰⁰;
each R¹⁰⁰ is independently selected from H, and C₁₋₆ alkyl;
ring C is selected from C₃₋₇ cycloaliphatic, C₆₋₁₀ aryl, 3 to 12-membered heterocyclyl, and 5 to 1-membered heteroaryl, each of which is optionally substituted with one or more R²;
each R² is independently selected from halo, —CN, —NO₂, —OR²⁰⁰, —OC(O)N(R²⁰⁰)₂, —OC(O)R²⁰⁰, —SR²⁰⁰, —S(O)R²⁰⁰, —SO₂R²⁰⁰, —S(O)N(R²⁰⁰)₂, —SO₂N(R²⁰⁰)₂, —C(O)R²⁰⁰, —CO₂R²⁰⁰, —C(O)N(R²⁰⁰)₂, —C(O)N(R²⁰⁰)OR²⁰⁰, —N(R²⁰⁰)₂, —N(R²⁰⁰)C(O)R²⁰⁰, —N(R²⁰⁰)CO₂R²⁰⁰, —N(R²⁰⁰)C(O)N(R²⁰⁰)₂, —N(R²⁰⁰)SO₂R²⁰⁰, C₁₋₆ alkyl and C₁₋₆ haloalkyl;
each R²⁰⁰ is independently selected from H and C₁₋₆ alkyl;
alternatively two adjacent R², taken together with the carbon atoms of ring C to which they are attached, form a 3 to 7-membered heterocyclyl;
ring D is selected from C₃₋₇ cycloaliphatic, C₆₋₁₀ aryl, 3 to 12-membered heterocyclyl and 5 to 10-membered heteroaryl, each of which is optionally substituted by one or more R¹¹;
each R¹¹ is independently selected from halo, —CN, —NO₂, —OR¹¹⁰, —SR¹¹⁰, —OC(O)R¹¹⁰, —OC(O)N(R¹¹⁰)₂, —S(O)R¹¹⁰, —SO₂R¹¹⁰, —S(O)N(R¹¹⁰)₂, —SO₂N(R¹¹⁰)₂, —N(R¹¹⁰)₂, —COR¹¹⁰, —CO₂R¹¹⁰, —N(R¹¹⁰)C(O)N(R¹¹⁰)₂, —C(O)N(R¹¹⁰)₂, —C(O)N(R¹¹⁰)OR¹¹⁰, C₁₋₆ alkyl and C₁₋₆ haloalkyl;
each R¹¹⁰ is independently selected from H and C₁₋₆ alkyl;
alternatively two adjacent R¹¹, taken together with the carbon atoms of ring D to which they are attached, form a 3 to 7-membered heterocyclyl;
alternatively one R¹⁰ and one R², taken together with their intervening atoms, form a 5- or 6-membered heterocyclyl ring;
R³ is selected from H, halo, C₁₋₆ alkyl, C₁₋₆ haloalkyl and C₃₋₇ cycloaliphatic;
R⁴ is selected from H, halo, —CN, —OR⁴⁰, C₁₋₆ alkyl and C₁₋₆ haloalkyl;
R⁴⁰ is selected from H, C₁₋₆ alkyl and C₁₋₆ haloalkyl;
R⁵ is selected from H, halo, C₁₋₆ alkyl and C₁₋₆ haloalkyl;
R⁶ is selected from H, halo, C₁₋₆ alkyl and C₁₋₆ haloalkyl;
R⁷ is H or C₁₋₆ alkyl optionally substituted with —OR⁷⁰ or N(R^{N})₂; and
R⁷⁰ is selected from H, halo and C₁₋₆ alkyl;
and wherein:
(i) at least one of R³, R⁴, R⁵ and R⁶ is not H;
(ii) when X is NH₂, then ring C is C₃₋₇ cycloaliphatic, 3 to 12-membered heterocyclyl or 5 to 10-membered heteroaryl, each of which is optionally substituted with one or more R²;
(iii) when X is NH₂ or —N(R^{N})—R¹, then R³, R⁵ and R⁶ are all H; and, when X is NH₂, and ring C is phenyl, then the phenyl substituted with one or more R²; and
(iv) when X is —N(R^{N})-ring D, then ring C and ring D are not both optionally-substituted C₆₋₁₀ aryl; and, when X is —N(R^{N})-ring D and ring D is 9-10 membered bicyclic heteroaryl or an optionally N-substituted piperindyl, then R³, R⁵ and R⁶ are all H.

2. The compound of claim 1, wherein the compound is represented by Formula (II): 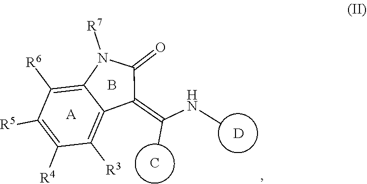
or a pharmaceutically acceptable salt thereof.

3. The compound of claim 1, wherein the compound is represented by Formula (III): 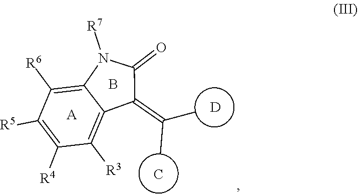
or a pharmaceutically acceptable salt thereof.

4. The compound of any one of claims 1-3, or a pharmaceutically acceptable salt thereof, wherein ring D is selected from phenyl, C₃₋₆ cycloaliphatic, 4 to 6-membered heterocyclyl and 5 to 6-membered heteroaryl, each of which is optionally substituted by one or more R¹¹.

5. The compound of any one of claims 1-3, or a pharmaceutically acceptable salt thereof, wherein ring D is a 5 to 6-membered heteroaryl optionally substituted by one or more R¹¹.

6. The compound of any one of claims 1-3, or a pharmaceutically acceptable salt thereof, wherein ring D is selected from azetindinyl, cyclobutyl, cyclohexyl, cyclopentyl, cyclopropyl, imidazolyl, morpholinyl, phenyl, piperidinyl, piperizinyl, pyrazolyl, pyrazinyl, pyridinyl, pyridazinyl, pyrimidinyl, pyrrolidinyl and tetrahydrofuranyl, each of which is optionally substituted by one or more R¹¹.

7. The compound of any one of claims 1-3, or a pharmaceutically acceptable salt thereof, wherein ring D is selected from pyrazolyl, pyridazinyl and pyrimidinyl, each of which is optionally substituted by one or more R¹¹.

8. The compound of any one of claims 1-7, or a pharmaceutically acceptable salt thereof, wherein each R¹¹ is independently —OH, —O—C₁₋₄alkyl, —O—C₁₋₄haloalkyl, —OC(O)R¹¹⁰, —OC(O)N(R¹¹⁰)₂, —CO₂R¹¹⁰, —C(O)N(R¹¹⁰)₂, C₁₋₆ alkyl or C₁₋₆ haloalkyl, and each R¹¹⁰ is independently H or C₁₋₆ alkyl.

9. The compound of any one of claims 1-7, or a pharmaceutically acceptable salt thereof, wherein each R¹¹ is independently —OH, —O—C₁₋₄alkyl, —CO₂R¹¹⁰ or C₁₋₆ alkyl, and each R¹¹⁰ is independently H or C₁₋₆ alkyl.

10. The compound of any one of claims 1-7, or a pharmaceutically acceptable salt thereof, wherein each R¹¹ is independently —OH, —CH₃ or CO₂H.

11. The compound of claim 1, wherein the compound is represented by Formula (IV): 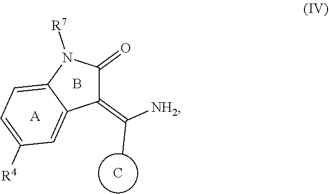
or a pharmaceutically acceptable salt.

12. The compound of claim 1, wherein the compound is represented by Formula (V) or (VI): 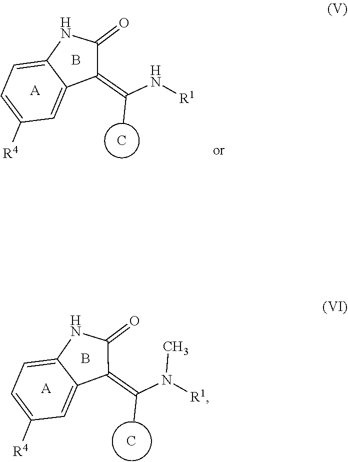
or a pharmaceutically acceptable salt thereof.

13. The compound of claim 1, wherein the compound is represented by Formula (VII): 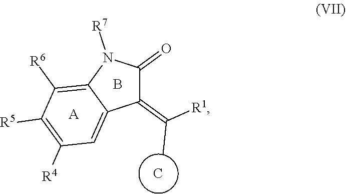
or a pharmaceutically acceptable salt thereof.

14. The compound of claim 1, wherein the compound is represented by Formula (VIII): 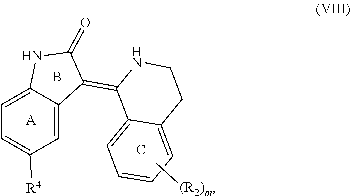
or a pharmaceutically acceptable salt thereof, wherein m is 0, 1, 2 or 3.

15. The compound of any one of claims 1, 12 and 13, or a pharmaceutically acceptable salt thereof, wherein:
R¹ is C₁₋₃ alkyl substituted with one or more R¹⁰;
each R¹⁰ is independently selected from halo, —OR¹⁵, —N(R¹⁰⁰)₂ and phenyl;
each R¹⁵ is independently selected from H, —CH₃ and —(CH₂CH₂O)ₙ—NHC(O)R¹⁰⁰;
n is 1, 2, 3, 4 or 5; and
each R¹⁰⁰ is independently H or —CH₃.

16. The compound of claim 15, or a pharmaceutically acceptable salt thereof, wherein each R¹⁰ is independently selected from F, —OH, —NH₂, —O—(CH₂CH₂O)ₙ—NHC(O)CH₃ and phenyl.

17. The compound of any one of claims 1-13, 15 and 16, or a pharmaceutically acceptable salt thereof, wherein ring C is selected from C₃₋₇ cycloaliphatic, C₆₋₁₀ aryl, 9-membered heterocyclyl and 5 to 9-membered heteroaryl, each of which is optionally substituted with one or more R².

18. The compound of any one of claims 1-13, 15 and 16, or a pharmaceutically acceptable salt thereof, wherein ring C is the 5 to 9-membered heteroaryl optionally substituted with one or more R².

19. The compound of any one of claims 1-13, 15 and 16, or a pharmaceutically acceptable salt thereof, wherein ring C is selected from benzofuranyl, 2,3-dihydrobenzo[b][1,4]dioxinyl, isoxazolyl, napthylenyl, phenyl, pyridinyl, pyrimidinyl, 1H-pyrrolo[2,3-b]pyridinyl and thiazolyl, each of which is optionally substituted with one or more R².

20. The compound of any one of claims 1-13, 15 and 16, or a pharmaceutically acceptable salt thereof, wherein ring C is phenyl optionally substituted with one or more R².

21. The compound of any one of claims 1-20, or a pharmaceutically acceptable salt thereof, wherein each R² is independently selected from halo, —CN, —OR²⁰⁰, —OC(O)N(R²⁰⁰)₂, —OC(O)R²⁰⁰, —N(R²⁰⁰)₂, —N(R²⁰⁰)C(O)R²⁰⁰, —N(R²⁰⁰)CO₂R²⁰⁰, —N(R²⁰⁰)SO₂R²⁰⁰, C₁₋₄ alkyl and C₁₋₄ haloalkyl,
and each R²⁰⁰ is independently selected from H and C₁₋₄ alkyl.

22. The compound of any one of claims 1-20, or a pharmaceutically acceptable salt thereof, wherein each R² is independently selected from halo, —OR²⁰⁰, —N(R²⁰⁰)₂, —N(R²⁰⁰)C(O)R²⁰⁰, —N(R²⁰⁰)CO₂R²⁰⁰, —N(R²⁰⁰)SO₂R²⁰⁰, C₁₋₃ alkyl and C₁ haloalkyl, and each R²⁰⁰ is independently selected from H and C₁₋₃ alkyl.

23. The compound of any one of claims 1-20, or a pharmaceutically acceptable salt thereof, wherein each R² is independently selected from F, Cl, Br, —OH, —OCH₃, —NH₂, —N(CH₃)₂, —NHC(O)H, —CH(CH₃)₂, —NHCO₂CH(CH₃)₂, —NHSO₂CH₃, —CH₃, —CHF₂ and —CF₃.

24. The compound of any one of claims 1-10 and 15-23, or a pharmaceutically acceptable salt thereof, wherein R³ is selected from H, halo, C₁₋₄ alkyl and C₁₋₄ haloalkyl.

25. The compound of any one of claims 1-10 and 15-23, or a pharmaceutically acceptable salt thereof, wherein R³ is H or —CH₃.

26. The compound of any one of claims 1-25, or a pharmaceutically acceptable salt thereof, wherein R⁴ is selected from H, halo, —OR⁴⁰, C₁₋₃ alkyl and C₁₋₃ haloalkyl, where R⁴⁰ is C₁ haloalkyl.

27. The compound of any one of claims 1-25, or a pharmaceutically acceptable salt thereof, wherein R⁴ is halo or a fluoromethoxy.

28. The compound of any one of claims 1-25, or a pharmaceutically acceptable salt thereof, wherein R⁴ is selected from H, F, Cl, Br, —OCH₃, —OCHF₂, —OCF₃, —CH₃, —CH₂CH₃, —CF₃ and —CH(CH₃)₂.

29. The compound of any one of claims 1-25, or a pharmaceutically acceptable salt thereof, wherein R⁴ is Br or —OCF₃.

30. The compound of any one of claims 1-10 and 15-29, or a pharmaceutically acceptable salt thereof, wherein:
R⁵ is selected from H, halo and C₁₋₄ alkyl;
R⁶ is selected from H, halo and C₁₋₄ alkyl; and
R⁷ is H or C₁₋₄ alkyl optionally substituted with —OCH₃ or N(CH₃)₂.

31. The compound of any one of claims 1-10 and 15-29, or a pharmaceutically acceptable salt thereof, wherein
R⁵ is selected from H, Cl and —CH₃;
R⁶ is selected from H, F and —CH₃; and
R⁷ is selected from H, —CH₃, —CH₂CH₂OCH₃ and —CH₂CH₂N(CH₃)₂.

32. A pharmaceutical composition comprising the compound of any one of claims 1-31 or a pharmaceutically acceptable salt thereof, and a pharmaceutically acceptable carrier or excipient.

33. A method for treating a subject with a polyQ-related neurodegenerative disorder, the method comprising administering to the subject an effective amount of the compound of any one of claims 1-31 or a pharmaceutically acceptable salt thereof, or with an effective amount of the pharmaceutical composition of claim 32.

34. The method of claim 33, wherein the limitations (i)-(iv) do not apply.

35. The method of claim 33 or 34, wherein the polyQ-related neurodegenerative disorder is selected from spinocerebellar ataxia type 1, spinocerebellar ataxia type 2, spinocerebellar ataxia type 3, spinocerebellar ataxia type 7, spinocerebellar ataxia type 12, spinocerebellar ataxia type 17, dentatorubral-pallidoluysian atrophy, Huntington's disease and spinal-bulbar muscular atrophy.
